(19) United States
(12) Patent Application Publication
(10) Pub. No.: US 2008/0242742 A1
(43) Pub. Date: Oct. 2, 2008

(54) Animal models of pancreatic adenocarcinoma and uses therefor

(75) Inventors: Ronald A. Depinho, Brookline, MA (US); Nabeel El-Bardeesy, Boston, MA (US); Andrew J. Aguirre, Brookline, MA (US); David A. Tuveson, Berwyn, PA (US)

Correspondence Address: FOLEY HOAG, LLP;PATENT GROUP, WORLD TRADE CENTER WEST, 155 SEAPORT BLVD, BOSTON, MA, 02110, US

(73) Assignees: Dana-Farber Cancer Institute, Inc., Boston, MA (US); The Trustees of the University of Pennsylvania, Philadelphia, PA (US)

(21) Appl. No.: 11/503,499

(22) Filed: Aug. 11, 2006

Related U.S. Application Data
(63) Continuation of application No. 11/266,777, filed on Nov. 3, 2005.
Application No. 11/266,777 is a continuation of application No. 10/998,227, filed on Nov. 24, 2004, now abandoned.
(60) Provisional application No. 60/525,464, filed on Nov. 26, 2003.

(51) Int. Cl. A01K 67/027; C12Q 1/68; A61K 35/00; C12N 15/11; C07K 14/00

(52) U.S. Cl. 514/789; 800/10; 435/6; 536/23.1; 530/350

(57) ABSTRACT

The present invention is based, at least in part, on the generation of an animal model of pancreatic adenocarcinoma which recapitulates the genetic and histological features of human pancreatic adenocarcinoma, including the initiation, maintenance, and progression of the disease. Accordingly, the present invention provides animal models of cancer, e.g., pancreatic adenocarcinoma, wherein an activating mutation of Kras has been introduced, and any one or more known or unknown tumor suppressor genes or loci, e.g., Ink4a/Arf, Ink4a, Arf, p53, Smad4/Dpc, Lkb1, Brca2, or Mlh1, have been misexpressed, e.g., have been misexpressed leading to decreased expression or non-expression. The animal models of the invention may be used, for example, to identify biomarkers of pancreatic cancer, to identify agents for the treatment or prevention of pancreatic cancer, and to evaluate the effectiveness of potential therapeutic agents.

### RELATED APPLICATIONS

This application is a continuation application of U.S. patent application Ser. No. 11/266,777, filed Nov. 3, 2005, which is a continuation of U.S. patent application Ser. No. 10/998,227, filed Nov. 24, 2004, which claims the benefit of U.S. Provisional Application Ser. No. 60/525,464, filed on Nov. 26, 2003, the entire contents of each application which are incorporated herein by reference.

### GOVERNMENT FUNDING

This invention was made at least in part with government support under grant no. ______ awarded by the National Institutes of Health (NIH). The government has certain rights in this invention.

### BACKGROUND OF THE INVENTION

Pancreatic ductal adenocarcinoma has a median survival of 6 months and a 5 year survival of less than 5%, making it one of the most lethal human cancers (Warshaw, A. L. and C. Fernandez-del Castillo (1992) N Engl J Med 326: 455-65). This poor prognosis relates to the uniformly advanced disease stage at the time of diagnosis and to its profound resistance to existing therapies. A number of key challenges must be addressed to permit improvements in patient outcome, including the need to understand more definitively the cellular origins of this disease, to elucidate the biological interactions of the tumor cell and stromal components, to determine the role of specific genetic lesions and their signaling surrogates in the initiation and progression of the tumor, and to uncover the basis for the intense therapeutic resistance of these cancers (Kern, S., et al. (2001) Cancer Res 61: 4923-32).

This malignancy is thought to arise from the pancreatic ducts on the basis of its histological and immunohistochemical relationship to this cell type (Solcia, E., et al. (1995) Tumors of the Pancreas. Armed Forces Institute for Pathology, Washington, D.C.). Consistent with a ductal origin, premalignant lesions, known as Pancreatic Intraepithelial Neoplasms (PanINs) that are thought to arise from the smaller pancreatic ducts, are found in close physical contiguity with advanced malignant tumors (Cubilla, A. L. and P. J. Fitzgerald (1976) Cancer Res 36: 2690-8; Hruban, R. H., et al. (2001) Am J Surg Pathol 25: 579-86). PanINs appear to progress toward increasingly atypical histological stages and display the accumulation of clonal genetic changes suggesting that they are precursors of ductal adenocarcinoma (Moskaluk, C. A., et al. (1997) Cancer Res 57:2140-3; Yamano, M., et al. (2000) Am J Pathol 156:2123-33; Luttges, J., H. et al. (2001) Am J Pathol 158: 1677-83; Klein, W. M., et al. (2002) Mod Pathol 15: 441-7). The cell-of-origin question is complicated by the developmental plasticity of the pancreas that enables transdifferentiation between cell lineages (Sharma, A., et al. (1999) Diabetes 48: 507-13; Meszoely, I. M., et al. (2001). Cancer J 7: 242-50; Bardeesy, N. and R. A. DePinho (2002) Nat Rev Cancer 2: 897-909). Acinar cells have been shown to undergo metaplastic conversion to duct-like cells, both in culture, and under a variety of stresses in vivo (Jhappan, C., et al. (1990) Cell 61: 1137-46; Sandgren, E. P., et al. (1991) Proc Natl Acad Sci USA 88: 93-7; Hall, P. A. and N. R. Lemoine (1992) J Pathol 166: 97-103; Rooman, I., et al. (2000) Diabetologia 43: 907-14). The development of pancreatic tumors with ductal features following a process of acinar-ductal metaplasia in transgenic mice expressing TGF-α in the acini has suggested a progenitor role for acinar cells in this malignancy (Meszoely, I. M., et al. (2001). Cancer J 7: 242-50; Wagner, M., et al. (2001) Genes Dev 15: 286-93). Other experimental studies have suggested that islets cells or a putative pancreatic stem cell population may also give rise to pancreatic adenocarcinomas (Yoshida, T. and D. Hanahan (1994) Am J Pathol 145: 671-84; Pour, P. M., et al. (2003) Mol Cancer 2: 13). Finally, it remains possible that pancreatic adenocarcinoma arises from any one of these differentiated cell types or from tissue stem cells and, rather, that specific genetic lesions dictate the tumor's phenotypic end-point regardless of the originating cellular compartment. This paradigm has been previously suggested in malignant glioma (Holland, E. C., et al. (1998) Genes Dev 12: 3675-85; Bachoo, R. M., et al. (2002) Cancer Cell 1: 269-77).

Although a series of gene mutations and pathways that characterize pancreatic ductal adenocarcinoma have been identified, the means to properly engineer such mutations or pathway alterations into an accurate model have remained elusive. The ideal animal model would have the same histological features (i.e., gradual emergence from normal pancreatic cells towards progressive abnormal ductal lesions known as PanINs, display of ductal cellular morphology and immunophenotype, invasive growth and metastasis) as in the human disease (Solcia, et al. (1995) Tumors of the Pancreas, Volume Fascicle 20 (Washington, D.C.: Armed Forces Institute for Pathology); Hruban, et al. (2000) Am J Pathol 156:1821). The generation of such a model has been a long sought objective. However, numerous previous modeling attempts have failed to recapitulate the human disease at all levels (Wei, et al. (2003) Int J Gastrointest Cancer 33:43; Kern, et al. (2001) Cancer Res 61:4923; Hotz, et al. (2000) Int J Colorectal Dis 15:136; Bardeesy, et al. (2002) Nat Rev Cancer 2:897; Standop, et al. (2001) Dig Dis 19:24).

### SUMMARY OF THE INVENTION

The present invention is based, at least in part, on the generation of a non-human animal model of pancreatic ductal adenocarcinoma which recapitulates the genetic and histological features of the human disease, including the initiation, maintenance, and progression of the disease. The present invention includes animal models of cancer, e.g., pancreatic ductal adenocarcinoma, e.g., animal models wherein an activating mutation of Kras has been introduced, and any one or more known or unknown tumor suppressor genes or loci, e.g., Ink4a/Arf, Ink4a, Arf, p53, Smad4/Dpc, Lkb1, Brca2, or Mlh1, have been misexpressed, e.g., have decreased expression or lack of expression by, e.g., deletion of all or a portion of the one or more genes encoding the tumor suppressor gene.

Accordingly, in one aspect, the invention provides non-human, e.g., rodent, e.g., mouse, animal models of pancreatic adenocarcinoma, comprising an activating mutation of KRAS and wherein one or more tumor suppressor genes or loci are misexpressed, e.g., conditionally misexpressed resulting in decreased expression or non-expression. In one embodiment, the misexpressed tumor suppressor gene is Ink4a/ARF. In another embodiment, the misexpressed tumor suppressor genes are Ink4a/ARF and p53 in combination. In another embodiment, the tumor suppressor gene that is misexpressed is selected from the group consisting of Ink4a/Arf, Ink4a, Arf p53, Smad4/Dpc, Lkb1, Brca2, and Mlh1. The animal may be homozygous or heterozygous for the one or more disrupted genes or loci. In another embodiment, the one or more tumor suppressor genes or loci may be disrupted by removal of DNA encoding all or part of the tumor suppressor protein.

In one embodiment, the activating mutation of KRAS is a Kras^{G12D} knock-in allele (LSL-Kras). In another embodiment, the activating mutation of KRAS is a Kras^{G12D} knock-in allele (LSL-Kras), and the tumor suppressor gene is INK4a/Arf. In still another embodiment, the non-human animal comprises Pdx1-Cre; LSL-Kras^{G12D}; Ink4a/Arf^{lox/lox}.

In another embodiment, the non-human animal model is a transgenic animal with a transgenic disruption of said one or more tumor suppressor genes or loci. In one embodiment, the pancreatic and duodenal homeobox gene 1 (Pdx1)-Cre transgene is used to delete said one or more tumor suppressor genes or loci in the pancreas.

In another aspect, the invention provides methods for identifying for a biomarker associated with pancreatic adenocarcinoma comprising comparing the presence, absence, or level of expression or activity of genes or proteins in a sample, e.g., a sample containing tissue, whole blood, serum, plasma, buccal scrape, saliva, urine, stool, bile, pancreatic cells or pancreatic tissue, from an animal model of pancreatic adenocarcinoma, versus the presence, absence, or level of expression or activity of genes or proteins in a sample, e.g., blood, e.g., serum, urine, stool, bile, pancreatic juice, pancreatic tissue or a pancreatic cell from a control wild-type animal, wherein the animal model comprises an activating mutation of KRAS and wherein one or more tumor suppressor genes or loci are misexpressed, and wherein a difference in the presence, absence, or expression or activity level of a gene or protein indicates that the gene or protein is a biomarker associated with pancreatic adenocarcinoma. The identified biomarker may be a diagnostic biomarker, a prognostic biomarker, or a pharmacogenomic biomarker.

Accordingly, in another aspect, the invention provides methods for identifying a pharmacogenomic biomarker which is expressed in conjunction with a therapy regime comprising comparing the presence, absence, or level of expression or activity of genes or proteins in a sample, e.g., blood, e.g., serum, urine, stool, bile, pancreatic juice, pancreatic tissue or a pancreatic cell from an animal model of pancreatic adenocarcinoma, versus the presence, absence, or level of expression or activity of genes or proteins in the a sample, e.g., blood, e.g., serum, urine, stool, bile, pancreatic juice, pancreatic tissue or a pancreatic cell from a control wild-type animal, wherein the animal model comprises an activating mutation of KRAS and wherein one or more tumor suppressor genes or loci are misexpressed, wherein said animal model is administered a therapy regime; and wherein a difference in the presence, absence, or expression or activity level of a gene or protein in the indicates that the gene or protein is a pharmacogenomic biomarker associated with pancreatic adenocarcinoma. In one embodiment, the animal model displays metastatic pancreatic tumors. In another embodiment, the animal model is asymptomatic for pancreatic adenocarcinoma. In one embodiment, the biomarker is selected from the group consisting of SEQ ID NOs. 23 and 24.

In one aspect, the invention provides a method for identifying a biomarker associated with pancreatic adenocarcinoma, comprising: a) performing profiling of the genome of cancer cells, wherein the cells are from an animal model of pancreatic adenocarcinoma, wherein the animal model comprises an activating mutation of KRAS and wherein one or more tumor suppressor genes or loci are misexpressed; b) performing segmentation analysis of profiles identified in step a); c) identifying loci; d) prioritizing said identified loci; and e) interrogating genes in the identified loci, to thereby identify a biomarker associated with pancreatic adenocarcinoma. In one embodiment, the interrogation of genes in the identified loci is based on gene expression data. In another embodiment, the interrogation of genes in the identified loci is based on in vitro screening assays.

In another aspect, the invention provides a method for identifying a locus associated with pancreatic adenocarcinoma, said method comprising the steps of: a) performing profiling of the genome of cancer cells, wherein said cells are from an animal model of pancreatic adenocarcinoma, wherein the animal model comprises an activating mutation of KRAS and wherein one or more tumor suppressor genes or loci are misexpressed; b) performing segmentation analysis of profiles identified in step a); c) identifying loci; and d) prioritizing said identified loci, to thereby identify a locus associated with pancreatic adenocarcinoma. In one embodiment, a biomarker is identified by the method.

In one embodiment, the profiling is performed using comparative genomic hybridization (CGH). In another embodiment, the cancer cells are derived from a pancreatic adenocarcinoma cell line or a pancreatic adenocarcinoma tumor.

Still another aspect of the invention provides a biomarker identified by the methods described herein, e.g., a nucleic acid biomarker or a protein biomarker.

In another aspect, the invention provides methods of identifying a gene or protein involved in stromal-tumor communication comprising comparing the presence, absence, or level of expression or activity of genes or proteins in a tumor from an animal model of pancreatic adenocarcinoma, versus the presence, absence, or level of expression or activity of genes or proteins in stroma from an animal model of pancreatic adenocarcinoma, wherein the animal model comprises an activating mutation of KRAS and wherein one or more tumor suppressor genes or loci are misexpressed, and wherein a difference in the presence, absence, or expression or activity level of a gene or protein indicates that the gene or protein is involved in stromal-tumor communication.

In yet another aspect, the invention provides methods of assessing whether a subject is afflicted with pancreatic adenocarcinoma, the method comprising comparing the presence, absence, or level of expression or activity of a biomarker identified by the methods described herein in a subject sample, e.g., blood, e.g., serum, urine, stool, bile, pancreatic juice, pancreatic tissue or pancreatic cell sample, and the presence, absence, or level of expression or activity of the biomarker in a control sample, e.g., blood, e.g., serum, urine, stool, bile, pancreatic juice, pancreatic tissue or pancreatic cell sample, wherein a difference in the presence, absence, or level of expression or activity of the biomarker in the subject sample and the normal level is an indication that the subject is afflicted with pancreatic adenocarcinoma. In one embodiment, the sample comprises cells obtained from the patent.

In still another aspect, the invention provides methods for monitoring the progression of pancreatic adenocarcinoma in a subject, the method comprising a) detecting in a subject sample at a first point in time, the presence, absence, or level of expression or activity of a biomarker identified by the methods described herein; b) repeating step a) at a subsequent point in time; and c) comparing the presence, absence, or level of expression or activity detected in steps a) and b), and therefrom monitoring the progression of pancreatic adenocarcinoma in the subject. In one embodiment, the sample comprises cells obtained from the patent. In another embodiment, between the first point in time and the subsequent point in time, the subject has undergone surgery to remove a tumor.

In a further aspect, the invention provides methods of assessing the efficacy of a test compound for inhibiting pancreatic adenocarcinoma in a subject, the method comprising comparing the presence, absence, or level of expression or activity of a biomarker in a first sample obtained from the subject and exposed to the test compound, wherein the biomarker is identified by the methods described herein, and the presence, absence, or level of expression or activity of the biomarker in a second sample obtained from the subject, wherein the sample is not exposed to the test compound, wherein a significantly a difference in the presence, absence, or level of expression or activity of the biomarker in the first sample, relative to the second sample, is an indication that the test compound is efficacious for inhibiting pancreatic adenocarcinoma in the subject. In one embodiment, the first and second samples are portions of a single sample obtained from the subject.

The invention also provides methods of assessing the efficacy of a therapy for inhibiting pancreatic adenocarcinoma in a subject, comprising comparing expression of a biomarker in the first sample obtained from the subject prior to providing at least a portion of the therapy to the subject, wherein the biomarker is identified by the methods described herein, and expression of the biomarker in a second sample obtained from the subject following provision of the portion of the therapy, wherein a significantly lower level of expression of the biomarker in the second sample, relative to the first sample, is an indication that the therapy is efficacious for inhibiting pancreatic adenocarcinoma in the subject.

In another aspect, the invention provides methods of selecting a composition for inhibiting pancreatic adenocarcinoma in a subject, the method comprising obtaining a sample comprising cancer cells from the subject; separately exposing aliquots of the sample in the presence of a plurality of test compositions; comparing expression of a biomarker in each of the aliquots, wherein the biomarker is identified by the methods described herein; and selecting one of the test compositions which induces a lower level of expression of the biomarker in the aliquot containing that test composition, relative to other test compositions.

In still another aspect, the invention provides methods of inhibiting pancreatic adenocarcinoma in a subject, the method comprising obtaining a sample comprising cancer cells from the subject; separately maintaining aliquots of the sample in the presence of a plurality of test compositions; comparing expression of a biomarker in each of the aliquots, where the biomarker is identified by the methods described herein; and administering to the subject at least one of the test compositions which induces a lower level of expression of the biomarker in the aliquot containing that test composition, relative to other test compositions.

In yet another aspect, the invention provides a kit for assessing whether a subject is afflicted with pancreatic adenocarcinoma, the kit comprising reagents for assessing expression of a biomarker identified by the methods described herein. Another aspect of the invention provides a kit for assessing the presence of pancreatic adenocarcinoma cells, the kit comprising a nucleic acid probe wherein the probe specifically binds with a transcribed polynucleotide corresponding to a biomarker identified by the methods described herein.

The invention also provides a kit for assessing the suitability of each of a plurality of compounds for inhibiting pancreatic adenocarcinoma in a subject, the kit comprising the plurality of compounds; and a reagent for assessing expression of a biomarker identified by the methods described herein.

In another aspect, the invention provides a kit for assessing the presence of human pancreatic adenocarcinoma cells, the kit comprising an antibody, wherein the antibody specifically binds with a protein corresponding to a biomarker identified by the methods described herein.

Another aspect of the invention provides methods of assessing the pancreatic cell carcinogenic potential of a test compound, the method comprising maintaining separate aliquots of pancreatic cells in the presence and absence of the test compound; and comparing expression of a biomarker in each of the aliquots, wherein the biomarker is identified by the methods described herein, wherein a significantly enhanced level of expression of the biomarker in the aliquot maintained in the presence of the test compound, relative to the aliquot maintained in the absence of the test compound, is an indication that the test compound possesses human pancreatic cell carcinogenic potential.

In a further aspect, the invention provides a kit for assessing the pancreatic cell carcinogenic potential of a test compound, the kit comprising pancreatic cells and a reagent for assessing expression of a biomarker, wherein the biomarker is identified by the methods described herein.

In still another aspect, the invention provides methods of identifying a compound that modulates pancreatic adenocarcinoma development, progression, and/or maintenance comprising administering a test compound to an animal model comprising an activating mutation of KRAS and wherein one or more tumor suppressor genes or loci are misexpressed, or a cell isolated therefrom; and determining the effect of the test compound on the initiation, maintenance, or progression of pancreatic adenocarcinoma in said animal model.

In yet another aspect, the invention provides methods for evaluating a potential therapeutic agent for the treatment or prevention of pancreatic adenocarcinoma comprising administering a test compound to an animal model comprising an activating mutation of KRAS and wherein one or more tumor suppressor genes or loci are misexpressed, or a cell isolated therefrom; and determining the effect of the test compound on the initiation, maintenance, or progression of pancreatic adenocarcinoma in said animal model. In one embodiment, the compound is selected from the group consisting of: a protein, a nucleic acid molecule, an antibody, a ribozyme, an antisense oligonucleotide, an siRNA, and an organic or non-organic small molecule.

The invention also provides methods of treating or preventing pancreatic adenocarcinoma in a subject having or at risk of developing pancreatic adenocarcinoma, comprising administering a compound identified by the methods described herein.

In yet another aspect, the invention provides isolated cells, or a purified preparation of cells from an animal model of pancreatic adenocarcinoma comprising an activating mutation of KRAS and wherein one or more tumor suppressor genes or loci are misexpressed. In one embodiment, the cell is isolated from pancreatic tissue from said animal model of pancreatic adenocarcinoma. In another embodiment, the cell is a epithelial, stomal, acinar, or ductal cell. In yet another embodiment, the cell is transgenic cell, e.g., a mouse cell.

In one aspect, the invention provides a method of assessing whether a subject is afflicted with pancreatic adenocarcinoma or at risk for developing pancreatic adenocarcinoma, the method comprising comparing the copy number of a minimal common region (MCR) in a subject sample to the normal copy number of the MCR, wherein said MCR is selected from the group consisting of the MCRs listed in Table 2, and wherein an altered copy number of the MCR in the sample indicates that the subject is afflicted with pancreatic adenocarcinoma or at risk for developing pancreatic adenocarcinoma. In one embodiment, the copy number is assessed by fluorescent in situ hybridization (FISH). In another embodiment, the copy number is assessed by quantitative PCR (qPCR). In one embodiment, the normal copy number is obtained from a control sample.

In another aspect the invention provides a method of assessing whether a subject is afflicted with pancreatic adenocarcinoma or at risk for developing pancreatic adenocarcinoma, the method comprising comparing: a) the amount, structure, and/or activity of a biomarker in a subject sample, wherein the biomarker is a biomarker which resides in an MCR listed in Table 2; and b) the normal amount, structure, and/or activity of the of the biomarker, wherein a significant difference between the amount, structure, and/or activity of the biomarker in the sample and the normal amount, structure, and/or activity is an indication that the subject is afflicted with pancreatic adenocarcinoma or at risk for developing pancreatic adenocarcinoma. In one embodiment, the amount of a biomarker is compared. In another embodiment, the structure of a biomarker is compared. In yet another embodiment, the activity of a biomarker is compared. In another embodiment, the amount of the biomarker is determined by determining the level of expression of the biomarker. In one embodiment, the biomarker is determined by determining copy number of the biomarker. In yet another embodiment, the normal amount/structure, and/or activity of the biomarker is obtained from a control sample. In one embodiment, the sample is selected from the group consisting of blood, urine, stool, bile, pancreatic cells or pancreatic tissue. In one embodiment, the copy number is assessed by comparative genomic hybridization (CGH). In a further embodiment, CGH is performed on an array. In one embodiment, the level of expression of the biomarker in the sample is assessed by detecting the presence in the sample of a protein corresponding to the biomarker. In another embodiment, the presence of the protein is detected using a reagent which specifically binds with the protein. In one embodiment, the reagent is selected from the group consisting of an antibody, an antibody derivative, and an antibody fragment. In a further embodiment, the level of expression of the biomarker in the sample is assessed by detecting the presence in the sample of a transcribed polynucleotide or portion thereof, wherein the transcribed polynucleotide comprises the biomarker. In one embodiment, the transcribed polynucleotide is an mRNA. In another embodiment, the transcribed polynucleotide is a cDNA. In a further embodiment, the step of detecting further comprises amplifying the transcribed polynucleotide. In one embodiment, the level of expression of the biomarker in the sample is assessed by detecting the presence in the sample of a transcribed polynucleotide which anneals with the biomarker or anneals with a portion of a polynucleotide wherein the polynucleotide comprises the biomarker, under stringent hybridization conditions.

In yet another aspect, the invention provides a method for monitoring the progression of pancreatic adenocarcinoma in a subject, the method comprising: a) detecting in a subject sample at a first point in time, the amount and/or activity of a biomarker, wherein the marker is a marker which resides in an MCR listed in Table 2; b) repeating step a) at a subsequent point in time; and c) comparing the amount and/or activity detected in steps a) and b), and therefrom monitoring the progression of pancreatic adenocarcinoma in the subject. In one embodiment, the sample is selected from the group consisting of blood, urine, stool, bile, pancreatic cells or pancreatic tissue. In one embodiment, the activity of a biomarker is determined. In another embodiment, the amount of a biomarker is determined. In a further embodiment, the amount of the biomarker is determined by determining the level of expression of the biomarker. In a further embodiment, the level of expression of the biomarker in the sample is assessed by detecting the presence in the sample of a protein corresponding to the biomarker. In yet a further embodiment, the presence of the protein is detected using a reagent which specifically binds with the protein. In one embodiment, the reagent is selected from the group consisting of an antibody, an antibody derivative, and an antibody fragment. In one embodiment, the level of expression of the biomarker in the sample is assessed by detecting the presence in the sample of a transcribed polynucleotide or portion thereof, wherein the transcribed polynucleotide comprises the biomarker. In a further embodiment, the transcribed polynucleotide is an mRNA. In another embodiment, the transcribed polynucleotide is a cDNA. In yet another embodiment, the step of detecting further comprises amplifying the transcribed polynucleotide. In another embodiment, the level of expression of the biomarker in the sample is assessed by detecting the presence in the sample of a transcribed polynucleotide which anneals with the biomarker or anneals with a portion of a polynucleotide wherein the polynucleotide comprises the biomarker, under stringent hybridization conditions. In one embodiment, the sample comprises cells obtained from the subject. In another embodiment, between the first point in time and the subsequent point in time, the subject has undergone treatment for pancreatic adenocarcinoma, has completed treatment for pancreatic adenocarcinoma, and/or is in remission.

One aspect of the invention provides a method of assessing the efficacy of a test compound for inhibiting pancreatic adenocarcinoma in a subject, the method comprising comparing: a) the amount and/or activity of a biomarker in a first sample obtained from the subject and maintained in the presence of the test compound, wherein the biomarker is a biomarker which resides in an MCR listed in Table 2; and b) the amount and/or activity of the biomarker in a second sample obtained from the subject and maintained in the absence of the test compound, wherein a significantly higher amount and/or activity of a biomarker in the first sample residing in an MCR which is deleted in pancreatic adenocarcinoma, relative to the second sample, is an indication that the test compound is efficacious for inhibiting pancreatic adenocarcinoma, and wherein a significantly lower amount and/or activity of a biomarker in the first sample residing in an MCR which is amplified in pancreatic adenocarcinoma, relative to the second sample, is an indication that the test compound is efficacious for inhibiting pancreatic adenocarcinoma in the subject. In one embodiment, the first and second samples are portions of a single sample obtained from the subject. In another embodiment, the first and second samples are portions of pooled samples obtained from the subject.

Another aspect of the invention provides a method of assessing the efficacy of a therapy for inhibiting pancreatic adenocarcinoma in a subject, the method comprising comparing: a) the amount and/or activity of a biomarker in the first sample obtained from the subject prior to providing at least a portion of the therapy to the subject, wherein the biomarker is a biomarker which resides in an MCR listed in Table 2, and b) the amount and/or activity of the biomarker in a second sample obtained from the subject following provision of the portion of the therapy, wherein a significantly higher amount and/or activity of the biomarker in the first sample residing in an MCR which is deleted in pancreatic adenocarcinoma, relative to the second sample, is an indication that the test compound is efficacious for inhibiting pancreatic adenocarcinoma and wherein a significantly lower amount and/or activity of the biomarker in the first sample residing in an MCR which is amplified in pancreatic adenocarcinoma, relative to the second sample, is an indication that the therapy is efficacious for inhibiting pancreatic adenocarcinoma in the subject.

Yet another aspect of the invention provides a method of selecting a composition capable of modulating pancreatic adenocarcinoma, the method comprising: a) obtaining a sample comprising pancreatic adenocarcinoma cells; b) contacting said cells with a test compound; and c) determining the ability of the test compound to modulate the amount and/or activity of a biomarker, wherein the biomarker is a biomarker which resides in an MCR listed in Table 2, thereby identifying a modulator of pancreatic adenocarcinoma. In one embodiment, the cells are isolated from an animal model of pancreatic adenocarcinoma. In another embodiment, the cells are from a pancreatic adenocarcinoma cell line. In yet another embodiment, the cells are from a subject suffering from pancreatic adenocarcinoma. In a further embodiment, the cells are from a pancreatic adenocarcinoma cell line originating from a pancreatic adenocarcinoma tumor.

One aspect of the invention provides a method of selecting a composition capable of modulating pancreatic adenocarcinoma, the method comprising: a) contacting a biomarker which resides in an MCR listed in Table 2 with a test compound; and b) determining the ability of the test compound to modulate the amount and/or activity of a biomarker which resides in an MCR listed in Table 2, thereby identifying a composition capable of modulating pancreatic adenocarcinoma. In one embodiment, the method further comprises administering the test compound to an animal model of pancreatic adenocarcinoma.

Another aspect of the invention provides a kit for assessing the ability of a compound to inhibit pancreatic adenocarcinoma, the kit comprising a reagent for assessing the amount, structure, and/or activity of a biomarker which resides in an MCR listed in Table 2.

One aspect of the invention provides a kit for assessing whether a subject is afflicted with pancreatic adenocarcinoma, the kit comprising a reagent for assessing the copy number of an MCR selected from the group consisting of the MCRs listed in Table 2.

Yet another aspect of the invention provides a kit for assessing whether a subject is afflicted with pancreatic adenocarcinoma, the kit comprising a reagent for assessing the amount, structure, and/or activity of a biomarker which resides in an MCR listed in Table 2.

One aspect of the invention provides a kit for assessing the presence of human pancreatic adenocarcinoma cells, the kit comprising an antibody or fragment thereof, wherein the antibody or fragment thereof specifically binds with a protein corresponding to a biomarker which resides in an MCR listed in Table 2.

Another aspect of the invention provides a kit for assessing the presence of pancreatic adenocarcinoma cells, the kit comprising a nucleic acid probe wherein the probe specifically binds with a transcribed polynucleotide corresponding to a biomarker which resides in an MCR listed in Table 2. In one embodiment, the nucleic acid probe is a molecular beacon probe.

One aspect of the invention provides a method of treating a subject afflicted with pancreatic adenocarcinoma comprising administering to the subject a modulator of amount and/or activity of a gene or protein corresponding to a biomarker which resides in an MCR listed in Table 2, thereby treating a subject afflicted with pancreatic adenocarcinoma.

Another aspect of the invention provides a method of treating a subject afflicted with pancreatic adenocarcinoma comprising administering to the subject a compound which inhibits the amount and/or activity of a gene or protein corresponding to a biomarker which resides in an MCR listed in Table 2 which is amplified in pancreatic adenocarcinoma, thereby treating a subject afflicted with pancreatic adenocarcinoma. In one embodiment, the compound is administered in a pharmaceutically acceptable formulation. In one embodiment, the compound is an antibody or an antigen binding fragment thereof, which specifically binds to a protein corresponding to said biomarker. In a further embodiment, the antibody is conjugated to a toxin. In yet another embodiment, the antibody is conjugated to a chemotherapeutic agent. In one embodiment, the compound is an RNA interfering agent which inhibits expression of a gene corresponding to said biomarker. In a further embodiment, the RNA interfering agent is an siRNA molecule or an shRNA molecule. In one embodiment, the compound is an antisense oligonucleotide complementary to a gene corresponding to said biomarker. In another embodiment, the compound is a peptide or peptidomimetic. In yet another embodiment, the compound is a small molecule which inhibits activity of said biomarker. In one embodiment, the small molecule inhibits a protein-protein interaction between a biomarker and a target protein. In one embodiment, the compound is an aptamer which inhibits expression or activity of said biomarker.

Another aspect of the invention provides a method of treating a subject afflicted with pancreatic adenocarcinoma comprising administering to the subject a compound which increases expression or activity of a gene or protein corresponding to a biomarker which resides in an MCR listed in Table 2 which is deleted in pancreatic adenocarcinoma, thereby treating a subject afflicted with pancreatic adenocarcinoma.

Yet another aspect of the invention provides a method of treating a subject afflicted with pancreatic adenocarcinoma comprising administering to the subject a protein corresponding to a biomarker which resides in an MCR listed in Table 2 which is deleted in pancreatic adenocarcinoma, thereby treating a subject afflicted with pancreatic adenocarcinoma. In one embodiment, the protein is provided to the cells of the subject, by a vector comprising a polynucleotide encoding the protein. In another embodiment, the compound is administered in a pharmaceutically acceptable formulation.

One aspect of the invention provides an isolated nucleic acid molecule, or fragment thereof, contained within an MCR selected from the MCRs listed in Table 2, wherein said nucleic acid molecule has an altered amount, structure, and/or activity in pancreatic adenocarcinoma.

Another aspect of the invention provides an isolated polypeptide encoded by the nucleic acid molecule.

### BRIEF DESCRIPTION OF THE DRAWINGS

FIGS. 1A-1E depict preinvasive ductal lesions arising in Pdx1-Cre; LSL-Kras^{G12D} mice. (A) Genetic progression model of human pancreatic adenocarcinoma. The cellular phenotypes of the increasing grades of ductal neoplastic lesions are indicated. Previous studies have catalogued the presence of genetic alterations at specific disease stages, as depicted in the temporal sequence. The thickness of the line corresponds to the frequency of a lesion. Loss-of-function events are depicted in red whereas gain-of-function lesions are shown in green. (B) Upper panel: H&E stain showing a normal islet (arrow) and duct (arrowheads) in the background of normal acinar tissue (asterisks) in a 12-week Pdx1-Cre; LSL-Kras^{G12D} mouse. An adjacent blood vessel (BV) is also indicated. Lower panel: Higher-power view of the single-layer cuboidal ductal epithelium. (C) PanIN-1 lesions detected in a 9-week old Pdx1-Cre; LSL-Kras^{G12D} mouse (H&E staining). Note PanIN lesions with mucinous columnar epithelium (arrows) and papillary architecture (dashed box). (D) Focus of ductal proliferation (dashed box) with prominent stromal response (asterisk) in a 12-week old Pdx1-Cre; LSL-Kras^{G12D} mouse (H&E staining). (E) Extensive PanIN lesions with a classical picture of intimately associated fibrotic stroma (asterisk) in the pancreas of a Pdx1-Cre; LSL-Kras^{G12D} mouse at 26-weeks of age (H&E staining).

FIGS. 2A-2K depict Ink4a/Arf deficiency promotes progression to invasive pancreatic adenocarcinoma. (A) Complete excision of the Ink4a/Arf locus in the pancreas with Pdx1-Cre. PstI Southern blot on pancreas (P) or spleen (S) DNA from Ink4a/Arf^{lox/+} mice that harbor or lack the Pdx1-Cre transgene. The wild-type or lox allele migrates at 9.0 kb and the recombined Ink4a/Arf-null allele corresponds to the 4.6 kb band. (B) Kaplan-Meier pancreatic tumor-free survival curve for Pdx1-Cre; LSL-Kras^{G12D}; Ink4a/Arf^{lox/lox} mice (denoted “Ink/Arf L/L”: n=26 mice) and control cohorts (denoted “ctrl”: all combinations of Pdx1-Cre, LSL-KRAS and Ink4a/Arf alleles, n=186 mice). Clinically, mice presented in a moribund state and were euthanized for autopsy. (C) Gross photograph of a pancreatic adenocarcinoma obstructing the common bile duct and causing dilation of the gall bladder (*). Jaundice is readily apparent in the abdominal skin (J). T=tumor; D=duodenum; L=Liver. Bar=0.6 cm. Dashed circle denotes the tumor. (D) Well-differentiated ductal adenocarcinoma observed in a Pdx1-Cre; LSL-Kras^{G12D}; Ink4a/Arf^{lox/lox} animal at 7.9 weeks of age. Glandular tumor cells (arrowheads) are surrounded by abundant stroma (*). (E) Poorly-differentiated adenocarcinoma arising in the same mouse as that from panel (D). Irregular, ill-formed formed glands (arrowheads) are present with mixture of highly mitotic, atypical tumor cells. (F) Region of tumor with sarcomatoid features from the same mouse as (D) and (E). (G-I) Regions of well-differentiated (G), poorly-differentiated (H) and sarcomatoid (I) tumor stained for the ductal marker, cytokeratin-19. (J) PAS+D stain for apical mucins in well-differentiated tumor cells. (K) Trichrome stain for collagen reveals fibrotic nature of tumor stroma.

FIGS. 3A-3F depict murine pancreatic tumors invade and metastasize. (A) Duodenal invasion by pancreatic ductal adenocarcinoma. Tumor (T), muscle wall (M, arrowhead) and intestinal epithelium (IE) are indicated. (B) High-magnification photomicrograph of a lymph node metastasis (T, arrowhead). LN denotes normal lymph node architecture. (C) Tumor cells (T) invading the stomach wall (M, arrowheads). Adjacent gastric epithelium is indicated (GE). (D) High-power micrograph of metastatic tumor cells (arrowheads) within a portal tract in the liver. Hepatocytes (H), portal vein (PV) and a reactive bile duct (B) are indicated. (E) Pancreatic tumor cells (T) invading the spleen. White pulp of the spleen is indicated (WP). (F) Immunohistochemical stain for cytokeratin-19 on tumor cells in panel E invading the spleen. Inset: Higher-power image of ck-19+ invading tumor cells.

FIGS. 4A-4F depict early-stage pancreatic lesions in Pdx1-Cre; LSL-Kras; Ink/Arf^{lox/lox} animals. (A) High-magnification view of a low-grade PanIN lesion (arrowhead) seen in a 5-week Pdx1-Cre; LSL-Kras^{G12D}; Ink4a/Arf^{lox/+} animal. (B) Low-grade preinvasive ductal lesion in a 3 week-old Pdx1-Cre; LSL-Kras^{G12D}; Ink4a/Arf^{lox/lox} mouse. (C) High-grade preinvasive ductal lesion in a Pdx1-Cre; LSL-Kras^{G12D}; Ink4a/Arf^{lox/lox} mouse at 4-weeks. (D) Early focus of pancreatic adenocarcinoma in a 4 week-old Pdx1-Cre; LSL-Kras^{G12D}; Ink4a/Arf^{lox/lox} mouse. Note both the ductal and anaplastic components of this early cancer.

(E) High-grade PanIN lesion in a 5 week Pdx1-Cre; LSL-Kras^{G12D}; Ink4a/Arf^{lox/lox} mouse. Serial sectioning through the entire pancreas at 10 μm intervals failed to discover any foci of adenocarcinoma in this animal. (F) High-grade PanIN lesion (asterisk) surrounded by anaplastic tumor cells in a 5-week Pdx1-Cre; LSL-Kras^{G12D} Ink4a/Arf^{lox/lox} mouse.

FIGS. 5A-5G depict the molecular analysis of murine pancreatic adenocarcinomas. (A) Ras activation assay. Lysates from wild-type pancreas (lanes 1 and 2), Pdx1-Cre; LSL-Kras^{G12D} pancreas (lanes 3 and 4) and from the murine pancreatic adenocarcinomas (lanes 5 and 6) affinity precipitated with Raf RBD agarose (Upstate) and then subjected to immunoblot analysis with anti-Ras antibodies. (B) PCR analysis of the Ink4a/Arf locus in murine pancreatic adenocarcinoma cell lines. Multiplex PCR was performed on DNA from the pancreatic cancer cell lines (lanes 3-16) with primers that amplify the Ink4a/Arf+ (lower band), Ink4a/Arf lox (middle band), and Ink4a/Arf− (upper band) alleles. DNA from Ink4a/Arf+/+(+/+, lanes 1 and 18) and Ink4a/Arf^{lox/lox} (L/L, lanes 2 and 17) mice served as controls. All cell lines show only the Ink4a/Arf− allele. (C) Immunoblot analysis of the tumor lysates. Membranes were immunoblotted for p16Ink4a, p19Arf, Smad4, and β-tubulin (as a loading control). Lysates from primary mouse embryonic fibroblasts (MEF, lane 1) served as a positive control. (D) Immunoblot analysis of p53 expression. Primary MEFs (lane 1) and p53−/− MEFs (lane 2), were positive and negative controls, respectively. (E) Induction of p53 and p21 in pancreatic adenocarcinoma cells by ionizing irradiation. Mouse pancreatic cancer cell lines were either untreated (−) or gamma irradiated (+) (lanes 1-8). Lysates were immunoblotted for p53, p21 and β-tubulin. MEFs with a mutant p53 allele (p53*) were a control for p53 overexpression. The tumors show modest expression of p53 compared to cells with mutant stabilized p53 and that ionizing radiation can effectively induce p53 and p21 in these tumor cells. (F) Amplification of the Kras gene and elevated Kras protein expression in a subset of pancreatic adenocarcinomas. The upper panel shows the relative Kras gene copy number as measured by quantitative real-time PCR. Wild-type specimens have a ratio of 1.0; (−) not done. The middle and lower panels show Western blot analysis of the corresponding Kras levels and the tubulin (tub) loading control, respectively. Lane 1 is a control MEF specimen. Lanes 2-15 are tumor cell line specimens. Lanes 10 and 14 show both high-level Kras gene amplification and protein overexpression. (G) The mutant Kras allele is amplified in tumors showing increased Kras gene copy number. RT-PCR/RFLP analysis was performed on pancreatic adenocarcinoma cell line RNA to evaluate the whether the wildtype and Kras^{G12D} alleles are expressed based on the Kras^{G12D}-specific HindIII site. PCR amplified cDNA was untreated (−) or digested with HindIII (+). Lanes 1-12 are tumor cell lines. Lanes 13 and 14 are control testes cDNA. All tumors express both alleles. Tumors 58 and 65 (lanes 2 and 4), corresponding to lanes 10 and 14 in FIG. (5F), show an increased relative ratio of the lower, Kras^{G12D} allele, consistent with amplification and overexpression of this mutant allele.

FIGS. 6A-6D depict the expression of EGFR and HER2 in pancreatic adenocarcinomas. (A, B) Immunohistochemistry with anti-EGFR (A) or anti-HER2 (B) antibodies shows robust expression of these proteins in the glandular regions of the tumors. (C, D) Immunohistochemistry for EGFR and HER2 reveals very weak or absent expression in the poorly differentiated regions of these tumors. Note that (C) and (D) were photographed from adjacent regions of the slides depicted in (A) and (B).

FIGS. 7A-7D depict the conditional targeting of exons 2 and 3 of the Ink4/Arf locus. (A) Exons 1β and 1α of Arf and Ink4a, respectively, are shown, as are the common exons 2 and 3. ES cells were targeted using the KO construct in which a loxP site was inserted in the EcoRV site between exon 1α and exon 2 and a neomycin cassette (Neo), flanked by Frt sites and a single loxP site, was inserted at the StuI site 3′ to exon 3. The Diptheria toxin gene (DT) served as a negative selection marker. Chimeric mice were crossed to the CAGG:Flpe strain to excise Neo in vivo, generating the Ink4a/Arf^{lox} allele. Cre-mediated excision deletes exons 2 and 3 of Ink4a/Arf^{lox}, disrupting both products of this locus. The restriction sites are StuI (S), SpeI (Sp), EcoRV (E) and PstI (P). A 3′ fragment (Probe A) was used following PstI restriction digests to screen for recombinant ES cell clones and to assess Cre-mediated deletion. (B) Southern blot of PstI digested DNA hybridized with probe A, showing the production of the 4.6 kb Ink4a/Arf null (−) allele following crosses of Ink4a/Arf^{lox/lox} mice with the EIIa-Cre general deleter strain. The wild-type allele is 9 kb. (C) Western blot analysis of p16^{Ink4a} and p19^{Arf} expression in mouse embryonic fibroblasts (MEFs). MEFs were prepared from two Ink4a/Arf^{+/+} and two Ink4a/Arf^{lox/lox} embryos and were subsequently either infected with retroviruses expressing Cre (Silver and Livingston 2001) (+, lanes 6-9) or were untreated (−, lanes 2-5). Ink4a/Arf^{-/-} MEFs (Serrano et al. 1996) were used a negative control for immunoreactivity. The non-specific (n.s.) band served to show equal loading. Untreated Ink4a/Arf^{lox/lox} MEFs show p16^{Ink4a} and p19^{Arf} expression at comparable levels to wildtype MEFs whereas as Ink4a/Arf^{lox/lox} MEFs exposed to Cre do not express either protein. (D) Ink4a/Arf^{lox/lox} (LL) and Ink4a/Arf^{+/+} (++) MEFs were either infected with Cre retroviruses (+Cre) or untreated and subjected to serial passage by the 3T3 protocol. Note that the Cre-treated Ink4a/Arf^{lox/lox} cultures showed immortal growth while all other cultures underwent passage-induced senescence.

FIGS. 8A-8D depict Pdx1-Cre; LSL-Kras^{G12D}; Ink4a/Arf^{lox/lox} tumors stain negative for markers of acinar or islet cell differentiation. (A and C) Immunohistochemistry for the acinar marker amylase in a region of well-differentiated ductal adenocarcinoma (A) and a focus of anaplastic change (C) for the same pancreatic tumor arising in a Pdx1-Cre; LSL-Kras^{G12D}; Ink4a/Arf^{lox/lox} mouse. Inset in (A): intense anti-amylase reactivity in preserved acinar tissue adjacent to the tumor. (B and D) Immunohistochemistry for the endocrine marker insulin in both well-differentiated ductal adenocarcinoma (B) and anaplastic tumor cells (D). Inset in (B): strong anti-insulin reactivity in an adjacent islet. Note negative staining of the apposed duct.

FIG. 9 depicts the Kaplan-Meier pancreatic tumor-free survival curve for Pdx1-Cre; LSL-Kras^{G12D}, Ink4a^{lox/+}; p53^{lox/+} mice (squares) and control cohorts (diamonds). Clinically, mice presented in a moribund state and were euthanized for autopsy.

FIG. 10 depicts the data from Eprogen ProteoSep system showing serum protein profiles of normal versus pancreatic tumor bearing mouse (LSL-Kras^{G12D}; Ink4a/Arf^{lox/lox} mice). The left panel depicts a PI/hydrophobicity plot comparing differences in protein abundance in control versus cancer bearing mice. Equivalent abundances appear white, tumor-associated increases appear light gray and those from normal animals are dark gray. The right panel depicts a tracing of proteins of a given PI and different hydrophobicity in control and cancer bearing animals. Note the highly overexpressed protein is a biomarker for pancreatic cancer (arrow).

FIG. 11 depicts the timeline of tumor progression in the Pdx1-Cre; LSL-Kras^{G12D}; Ink4a/Arf^{lox/lox} model and indicates the points at which specimens were collected for this analysis.

FIG. 12 depicts the genomic complexity of Ink4a/Arf versus p53 mouse pancreatic adenocarcinomas. Pdx1-Cre; LSL-Kras^{G12D} mic harboring either the Ink4a/Arf or p53 conditional tumor suppressor genes were allowed to generate pancreatic adenocarcinomas and low passage cell lines were derived from these tumors. Genomic DNA from these cell lines was analyzed by array-CGH according to standard protocols. The genomic complexity of tumors harboring Ink4a/Arf or p53 tumor suppressor mutations is plotted as a density function, with the X axis corresponding to the array-CGH value (Log₂ of the fluorescence ratio between tumor and normal DNA). Array-CGH values above “0” correspond to amplified or gained segments of tumor genome and those below “0” correspond to lost or deleted segments of tumor genome. A greater number of lost or gained segments are seen in tumors with p53 mutations.

FIGS. 13A and 13B depict cancer-specific expression of telomerase-associated protein and of the protein, ANKT, identified by proteomics profiling of serum from the Pdx1-Cre; LSL-Kras^{G12D}; Ink4a/Arf^{lox/lox} model of pancreatic adenocarcinoma (13A), and the sequence of specific peptides with high correlation with the disease state identified in these analyses (13B).

### DETAILED DESCRIPTION OF THE INVENTION

The present invention is based, at least in part, on the generation of non-human animal models of pancreatic adenocarcinoma which recapitulate the genetic and histological features of human pancreatic adenocarcinoma, including the initiation, maintenance, and progression of the disease. Accordingly, the present invention provides animal models of cancer, e.g., pancreatic adenocarcinoma, wherein an activating mutation of Kras has been introduced, and any one or more known or unknown tumor suppressor genes or loci, e.g., Ink4a/Arf, Ink4a, Arf, p53, Smad4/Dpc, Lkb1, Brca2, or Mlh1, have been misexpressed, e.g., have been misexpressed leading to decreased expression or non-expression. In one embodiment, misexpression of one or more tumor suppressor genes or loci is accomplished by a conditional allele which may be activated or deleted in specific cell types by the tissue-specific expression of Cre recombinase. In one embodiment, Ink4a/Arf is misexpressed in combination with the activation of Kras. In another embodiment, Ink4a/Arf and p53 are misexpressed in combination with the activation of Kras.

In particular, the present inventors have shown that activation of Kras in combination with misexpression of one or more tumor suppressor genes or loci, e.g., Ink4a/Arf, potently induces pancreatic adenocarcinoma, whereas either genetic lesion alone is insufficient for production of advanced malignant disease. Animal models have been engineered to bear both a pancreas-specific Cre-mediated mutant Kras allele (Kras^{G12D}) (Jackson, et al. (2001) Genes Dev. 15:3243) and a deletion of a conditional Ink4a/Arf allele (Ink4a/Arf^{lox}). The Kras allele is a ‘knock-in,’ i.e., it is controlled by its endogenous promoter. The Kras allele, Kras^{G12D}, carries an activating mutation (G12D), which results in the constitutive expression of Kras. Therefore, in the animal model, Kras is expressed at a level that mimics expression of the gene in human pancreatic adenocarcinoma. For Cre recombinase expression, the Pdx1-Cre transgene (Gu G. et al. (2002) Development 129, 2447-2457), which produces Cre activity in all the acinar, islet and duct cells and deletes loxP containing alleles in all pancreatic lineages, was employed. Kras is therefore activated at endogenous levels and Ink4a/Arf is deleted specifically in all cells of the pancreas.

Animals bearing the combination of these mutant alleles develop focal premalignant ductal lesions, termed pancreatic intraepithelial neoplasias (PanINs, as used herein) which rapidly and faithfully progress to highly aggressive, invasive and metastatic tumors which ultimately result in death of the animals by 11 weeks of age.

The evolution of these tumors bears striking resemblance to the human disease, possessing a proliferative stromal component and ductal lesions with a propensity to advance to a poorly differentiated state. The tumors arise with similar histological progression and identical immunohistochemical profiles to the human cancer. For example, the cancers arise from premalignant ductal lesions associated with Kras activation (PanINs) that progress to adenocarcinoma in conjunction with Ink4a/Arf deletion and express ductal but not endocrine or exocrine markers. The tumors arise extremely rapidly (all between 7 and 11 weeks) and with highly reproducible histology.

The rapid and narrow window within which the tumors, e.g., pancreatic adenocarcinomas, arise in the animal models of the invention greatly facilitates the identification of biomarkers and therapeutics for pancreatic adenocarcinoma, and the preclinical testing of compounds, as described herein. Accordingly, the present invention provides uses for the animal models of cancer, e.g., pancreatic adenocarcinoma, described herein, such as for example, biomarker discovery and screening assays. The biomarkers may be for biomarkers for disease, e.g., early disease markers or disease markers which identify various stages of disease including tumor maintenance and progression.

Furthermore, based on the high degree of similarity between pancreatic cancer displayed by the animal models of the invention and the human disease, the present invention also includes methods of using the animal models described herein and use of these biomarkers described herein in methods for, e.g., detection of pancreatic adenocarcinoma or identification of stage of cancer progression in a subject having or at risk for pancreatic adenocarcinoma.

In one embodiment, the identification of biomarkers is based on serum proteomics analyses of the animal models of the present invention compared to control animals (see, e.g., Example 2). In another embodiment, the identification of biomarkers is based on comparative genomic analyses of tumors from the animal models of the present invention (see, e.g., Example 3).

Accordingly, the present invention provides specific regions of the genome (referred to herein as minimal common regions (MCRs)), of recurrent copy number change which are contained within certain chromosomal regions (loci) and are associated with cancer. These MCRs were identified using a novel cDNA or oligomer-based platform and bioinformatics tools which allowed for the high-resolution characterization of copy-number alterations in the pancreatic cancer genome, e.g., the pancreatic adenocarcinoma genome.

To arrive at the MCRs, array comparative genomic hybridization (array-CGH) was utilized to define copy number aberrations (CNAs) (gains and losses of chromosomal regions) in pancreatic adenocarcinoma cell lines and tumor specimens.

The amplification or deletion of the MCRs identified herein correlate with the presence of cancer, e.g., pancreatic cancer and other epithelial cancers. Furthermore, analysis of copy number and/or expression levels of the genes residing within each MCR leads to the identification of individual biomarkers and combinations of biomarkers, the increased and decreased expression and/or increased and decreased copy number of which correlate with the presence and/or absence of cancer, e.g., pancreatic cancer, e.g., pancreatic adenocarcinoma in a subject.

Accordingly, methods are provided herein for detecting the presence of cancer in a sample, the absence of cancer in a sample, and other characteristics of cancer that are relevant to prevention, diagnosis, characterization, and therapy of cancer in a subject by evaluating alterations in the amount, structure, and/or activity of a biomarker. For example, evaluation of the presence, absence or copy number of the MCRs identified herein, or by evaluating the copy number, expression level, protein level, protein activity, presence of mutations (e.g., substitution, deletion, or addition mutations) which affect activity of the biomarker, or methylation status of any one or more of the biomarkers within the MCRs, is within the scope of the invention.

Methods are also provided herein for the identification of compounds which are capable of inhibiting cancer in a subject, and for the treatment, prevention, and/or inhibition of cancer using a modulator, e.g., an agonist or antagonist, of a gene or protein biomarker of the invention.

Although the MCRs and biomarkers described herein were identified in pancreatic cancer samples, the methods of the invention are in no way limited to use for the prevention, diagnosis, characterization, therapy and prevention of pancreatic cancer, e.g., the methods of the invention may be applied to any cancer, as described herein.

The present invention also provides screening methods using the animal models described herein or cells or cell lines derived from these animal models, for the identification of therapeutics for the treatment and/or prevention of pancreatic adenocarcinoma, e.g., molecularly targeted therapeutics that prevent or inhibit the malignant growth of a tumor.

In another aspect, the present invention provides methods of using the animal models described herein as a model system, e.g., a preclinical model system, for evaluation of potential therapeutic agents for the treatment and/or prevention of pancreatic adenocarcinoma. Based on the progression of pancreatic adenocarcinoma in the animal models of the invention, e.g., the presence of very early premalignant lesions (PanIN-1) at 3 weeks, more advanced premalignant lesions (PanIN-2) at 4 weeks and the presence of small pancreatic adenocarcinomas by 5 weeks, rapid measurement of the clinical impact of chemopreventative agents on disease onset and progression and of chemotherapeutics and disease response and cure is possible.

In yet another aspect, the invention also provides for the use of the animal models of the invention for the generation of cell lines which may be used to study the disease biology of pancreatic adenocarcinoma, e.g., for studies of heterotypic tumor-stroma interaction and identification of Kras in tumor maintenance program.

The genetically comparable, early passage mouse cell lines of the invention are useful for understanding the disease biology by, for example, studies of the basis for the heterotypic interactions between tumor and stroma using co-culture, gene expression profiling and manipulation of specific gene expression in either cell type (Olumi, A. F., et al. (1999) Cancer Res 59, 5002-5011; Tlsty, T. D., and Hein, P. W. (2001) Curr Opin Genet Dev 11, 54-59).

The cell lines of the invention may also be used for the discovery of new drug targets that disrupt the tumor-stromal symbiosis, such as, for example, compounds which not only target tumors cells directly but also exert an indirect effect by suppressing growth and survival signals elaborated by the microenvironments' interaction with the tumor cells.

The cell lines of the invention may also be used for identifying the KRAS transcriptional program and signaling surrogates. These cell lines are also be useful in the identification of the Kras oncogenic program and proteomics profile. The importance of KRAS for the sustained growth of the tumor cells can be evaluated by expression knock-down using RNAi techniques followed by orthotopic injection in SCID mice. This would allow 1) a determination of whether KRAS or its signaling surrogates are suitable drug targets, 2) identification of specific KRAS signaling surrogates (by expression profiling cells with intact or disrupted KRAS expression) that would serve as potential novel therapeutic targets, 3) determination of proteomics signatures of activated KRAS. The availability of the signature expression profile and proteomics profile provides powerful resources in the evaluation of drug efficacy and specificity directed towards KRAS or its signaling surrogates.

In another embodiment, the highly reproducible and rapid evolution of cancer in the animal models of the invention also make them suitable for conducting crosses to other inbred strains to identify possible modifiers to their cancer and for genetic tests of the contribution of specified proteins to tumorigenesis. The disclosed animals can also be used as research tools to determine genetic and physiological features of pancreatic cancer.

Various aspects of the invention are described in further detail in the following subsections:

### I. DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The terms “tumor” or “cancer” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, pancreatic cancer, e.g., pancreatic adenocasrcinoma, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like.

The term “pancreatic cancer” as used herein, includes adenomas, adenocarcinomas, gastrinomas, somatostatinomas, insulinomas and glucagonomas of the pancreas.

As used herein, the term “adenocarcinoma” is carcinoma that develops in the lining or inner surface of an organ and is derived from glandular tissue or in which the tumor cells form recognizable glandular structures.

As used interchangeably herein, the terms, “pancreatic adenocarcinoma,” or “pancreatic ductal adenocarcinoma” is an adenocarcinoma of the pancreas. In one embodiment, pancreatic adenocarcinomas arise from the progression of lesions that occur in the pancreatic ducts (pancreatic intraepithelial neoplasia, referred to herein as “PanIN”).

As used herein, a “transgenic animal” includes an animal, e.g., a non-human mammal, e.g., a primate, a swine, a goat, a sheep, a dog, a cow, a chicken, an amphibian, or a rodent, e.g., mouse, in which one or more, and preferably essentially all, of the cells of the animal include a transgene. The transgene is introduced into the cell, directly or indirectly by introduction into a precursor of the cell, e.g., by microinjection, transfection or infection, e.g., by infection with a recombinant virus. The term genetic manipulation includes the introduction of a recombinant DNA molecule. This molecule may be integrated within a chromosome, or it may be extrachromosomally replicating DNA.

As used herein, the term “rodent” refers to all members of the phylogenetic order Rodentia.

As used herein, the term “misexpression” includes a non-wild-type pattern of gene expression. Expression, as used herein, includes transcriptional, post transcriptional, e.g., mRNA stability, translational, and post translational stages. Misexpression includes: expression at non-wild-type levels, i.e., over or under expression; a pattern of expression that differs from wild-type in terms of the time or stage at which the gene is expressed, e.g., increased or decreased expression (as compared with wild-type) at a predetermined developmental period or stage; a pattern of expression that differs from wild-type in terms of decreased expression (as compared with wild-type) in a predetermined cell type or tissue type, e.g., pancreatic tissue; a pattern of expression that differs from wild-type in terms of the splicing size, amino acid sequence, post-transitional modification, or biological activity of the expressed polypeptide; a pattern of expression that differs from wild-type in terms of the effect of an environmental stimulus or extracellular stimulus on expression of the gene, e.g., a pattern of increased or decreased expression (as compared with wild-type) in the presence of an increase or decrease in the strength of the stimulus. Misexpression includes any expression from a transgenic nucleic acid. Misexpression includes the lack or non-expression of a gene or transgene, e.g., that can be induced by a deletion of all or part of the gene or its control sequences.

For example, misexpression of the gene encoding one or more tumor suppressor proteins, e.g., the Ink4a/Arf protein, may be caused by disruption of the tumor suppressor gene, e.g., the Ink4a/Arf gene. In one embodiment, a tumor suppressor gene, e.g., the Ink4a/Arf gene is disrupted through removal of DNA encoding all or part of the protein. In another embodiment, the animal can be heterozygous or homozygous for a misexpressed tumor suppressor gene, e.g., the Ink4a/Arf gene, e.g., it can be a transgenic animal heterozygous or homozygous for a tumor suppressor gene, e.g., an Ink4a/Arf transgene. In another embodiment, the animal is a transgenic mouse with a transgenic disruption of a tumor suppressor gene, e.g., the Ink4a/Arf gene, preferably an insertion or deletion, which inactivates the gene product. In a preferred embodiment, the animal or cell of the invention carries one or more tumor suppressor transgenes, e.g., an Ink4a/Arf transgene, and a transgene in Kras, e.g., Kras^{G12D} or an Ink4a/Arf transgene, a p53 transgene, and a transgene in Kras, e.g., Kras^{G12D}. In another embodiment, the animal or cell of the invention carries a tumor suppressor transgene which is selected from the group consisting of Ink4a, Arf p53, Smad4/Dpc, Lkb1, Brca2, or Mlh1.

As used herein, the term “knockout” refers to an animal or cell therefrom, in which the insertion of a transgene disrupts an endogenous gene in the animal or cell therefrom. This disruption can essentially eliminate, for example, Ink4a/Arf in the animal or cell.

As used herein, the term “knock-in” refers to an animal or cell therefrom, in which the insertion of a transgene disrupts an endogenous gene in the animal or cell therefrom and results in the alteration of gene function, e.g., expression level or expression pattern which differs from the wild-type expression level or expression pattern, and does not result in the loss of function of that gene.

The terms knockout and knock-in are also intended to refer to an animal or cell therefrom in which gene expression is modulated (e.g., disrupted or altered) in a conditional manner, e.g., “conditional knock-out” and/or a “conditional knock-in” system, which can also be used to create cells for use in screening assays. For example, a tetracycline-regulated system for conditional alteration of a gene as described in WO 94/29442 and U.S. Pat. No. 5,650,298 (incorporated herein by reference) can be used to create cells, or animals from which cells can be isolated, altered in a controlled manner through modulation of the tetracycline concentration in contact with the cells. In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236 and U.S. Pat. No. 4,959,317 (the contents of which are expressly incorporated herein by reference). Another example of a recombinase system is the FLP recombinase system of Saccharomyces cerevisiae (O'Gorman et al., 1991, Science 251:1351-1355, the contents of which are expressly incorporated herein by reference). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

In one embodiment, a knock-out animal of the invention carries a conditional knock-out of a tumor suppressor gene, e.g., p53 or Ink4a/Arf. For example, the conditional Ink4a/Arf allele (Ink4a/Arf^{lox}) was engineered to sustain Cre-mediated excision of exons 2 and 3, thereby eliminating both p16^{Ink4A} and p19^{Arf} proteins in a tissue specific manner (see Example 1). In another embodiment, a knock-in animal of the invention carries the conditional Kras^{G12D} knock-in allele (LSL-Kras) in which the LSL-Kras^{G12D} allele is expressed at endogenous levels following Cre-mediated excision of a transcriptional stopper element (Jackson, et al. (2001) Genes Dev. 15:3243). In another embodiment, a knock-in transgene harbors an activating mutation of a gene, for example Kras^{G12D}. As used herein an “an activating mutation” is a change in the sequence of a gene that results in the constituitive expression of that gene. For example, in vivo, tumorigenesis results in mutations in the coding sequence of Kras which are associated with decreased GTPase activity and constitutive signaling of Kras.

In another aspect, the invention features a nucleic acid molecule which, when introduced into an animal or cell, results in the misexpression of one or more tumor suppressor genes or loci, e.g., the Ink4a/Arf gene or the p53 gene in the animal or cell. In preferred embodiments, the nucleic acid molecule includes tumor suppressor genes or loci, e.g., INK4a/Arf, p53, Ink4a, Arf, p53, Smad4/Dpc, Lkb1, Brca2, or Mlh1, nucleotide sequence which includes a disruption, e.g., an insertion or deletion, e.g., the insertion of a marker sequence. The nucleotide sequence of the wild-type INK4a is known in the art and described in, for example, Genebank, gi:6753389 (SEQ ID NO.:1); the nucleotide sequence of the wild-type p53 is known in the art and described in, for example, Genebank, gi:8400737 (SEQ ID NO.:20)); the nucleotide sequence of the wild-type SMAD4 is known in the art and described in, for example, Genebank, gi:31543223 (SEQ ID NO.:21); the nucleotide sequence of the wild-type Lkb1 is known in the art and described in, for example, Genebank, gi: 7106424 (SEQ ID NO.:22); the nucleotide sequence of the wild-type Mlh1 is known in the art and described in, for example, Genebank, gi:19387851 (SEQ ID NO.:25); the nucleotide sequence of the wild-type Brca2 is known in the art and described in, for example, Genebank, gi:6857764 (SEQ ID NO.:26); the nucleotide sequence of the wild-type Arf1 is known in the art and described in, for example, Genebank, gi:31560734 (SEQ ID NO.:27); the contents of each are incorporated herein by reference.

As used herein, “disruption of a gene” refers to a change in the gene sequence, e.g., a change in the coding region. Disruption includes: insertions, deletions, point mutations, and rearrangements, e.g., inversions. The disruption can occur in a region of the native gene or locus, e.g., the native Ink4a/Arf or p53 DNA sequence (e.g., one or more exons) and/or the promoter region of the gene so as to decrease or prevent expression of the gene in a cell as compared to the wild-type or naturally occurring sequence of the gene. The “disruption” can be induced by classical random mutation or by site directed methods. Disruptions can be transgenically introduced. The deletion of an entire gene is a disruption. In one embodiment, disruptions reduce tumor suppressor gene or loci expression or activity levels e.g., Ink4a/Arf or p53 expression or activity levels, to about 50% of wild-type or in heterozygotes or essentially eliminate tumor suppressor gene or loci, e.g., Ink4a/Arf or p53 expression or activity, in homozygotes.

As used herein, the term “transgenic cell” refers to a cell containing a transgene.

As used herein, the term “marker sequence” refers to a nucleic acid molecule that (a) is used as part of a nucleic acid construct (e.g., the targeting construct) to disrupt the expression of the gene of interest (e.g., the Ink4a/Arf gene) and (b) is used to identify those cells that have incorporated the targeting construct into their genome. For example, the marker sequence can be a sequence encoding a protein which confers a detectable trait on the cell, such as an antibiotic resistance gene, e.g., neomycin resistance gene, or an assayable enzyme not typically found in the cell, e.g., alkaline phosphatase, horseradish peroxidase, luciferase, beta-galactosidase and the like.

A “minimal common region (MCR),” as used herein, refers to a contiguous chromosomal region which displays either gain and amplification (increased copy number) or loss and deletion (decreased copy number) in the genome of a cancer. An MCR includes at least one nucleic acid sequence which has increased or decreased copy number and which is associated with a cancer. The MCRs of the instant invention include, but are not limited to, those set forth in Table 2.

A “biomarker” is a gene or protein which may be altered, wherein said alteration is associated with cancer. The alteration may be in amount, structure, and/or activity in a cancer tissue or cancer cell, as compared to its amount, structure, and/or activity, in a normal or healthy tissue or cell (e.g., a control), and is associated with a disease state, such as cancer. For example, a biomarker of the invention which is associated with cancer may have altered copy number, expression level, protein level, protein activity, or methylation status, in a cancer tissue or cancer cell as compared to a normal, healthy tissue or cell. Furthermore, a “biomarker” includes a molecule whose structure is altered, e.g., mutated (contains an allelic variant), e.g., differs from the wild type sequence at the nucleotide or amino acid level, e.g., by substitution, deletion, or addition, when present in a tissue or cell associated with a disease state, such as cancer.

The term “altered amount” of a biomarker or “altered level” of a biomarker refers to increased or decreased copy number of a biomarker or chromosomal region, e.g., MCR, and/or increased or decreased expression level of a particular biomarker gene or genes in a cancer sample, as compared to the expression level or copy number of the biomarker in a control sample. The term “altered amount” of a biomarker also includes an increased or decreased protein level of a biomarker in a sample, e.g., a cancer sample, as compared to the protein level of the biomarker in a normal, control sample. Furthermore, an altered amount of a biomarker may be determined by detecting the methylation status of a biomarker, as described herein, which may affect the expression or activity of a biomarker.

The amount of a biomarker, e.g., expression or copy number of a biomarker or MCR, or protein level of a biomarker, in a subject is “significantly” higher or lower than the normal amount of a biomarker or MCR, if the amount of the biomarker is greater or less, respectively, than the normal level by an amount greater than the standard error of the assay employed to assess amount, and preferably at least twice, and more preferably three, four, five, ten or more times that amount. Alternately, the amount of the biomarker or MCR in the subject can be considered “significantly” higher or lower than the normal amount if the amount is at least about two, and preferably at least about three, four, or five times, higher or lower, respectively, than the normal amount of the biomarker or MCR.

The “copy number of a gene” or the “copy number of a biomarker” refers to the number of DNA sequences in a cell encoding a particular gene product. Generally, for a given gene, a mammal has two copies of each gene. The copy number can be increased, however, by gene amplification or duplication, or reduced by deletion.

The “normal” copy number of a biomarker or MCR or “normal” level of expression of a biomarker is the level of expression, copy number of the biomarker, or copy number of the MCR, in a biological sample, e.g., a sample containing tissue, whole blood, serum, plasma, buccal scrape, saliva, urine, stool, bile, pancreatic cells or pancreatic tissue, from a subject, e.g., a human, not afflicted with cancer.

The term “altered level of expression” of a biomarker or MCR refers to an expression level or copy number of a biomarker in a test sample e.g., a sample derived from a subject suffering from cancer, that is greater or less than the standard error of the assay employed to assess expression or copy number, and is preferably at least twice, and more preferably three, four, five or ten or more times the expression level or copy number of the biomarker or MCR in a control sample (e.g., sample from a healthy subjects not having the associated disease) and preferably, the average expression level or copy number of the biomarker or MCR in several control samples. The altered level of expression is greater or less than the standard error of the assay employed to assess expression or copy number, and is preferably at least twice, and more preferably three, four, five or ten or more times the expression level or copy number of the biomarker or MCR in a control sample (e.g., sample from a healthy subjects not having the associated disease) and preferably, the average expression level or copy number of the biomarker or MCR in several control samples.

An “overexpression” or “significantly higher level of expression or copy number” of a biomarker or MCR refers to an expression level or copy number in a test sample that is greater than the standard error of the assay employed to assess expression or copy number, and is preferably at least twice, and more preferably three, four, five or ten or more times the expression level or copy number of the biomarker or MCR in a control sample (e.g., sample from a healthy subject not afflicted with cancer) and preferably, the average expression level or copy number of the biomarker or MCR in several control samples.

An “underexpression” or “significantly lower level of expression or copy number” of a biomarker or MCR refers to an expression level or copy number in a test sample that is greater than the standard error of the assay employed to assess expression or copy number, but is preferably at least twice, and more preferably three, four, five or ten or more times less than the expression level or copy number of the biomarker or MCR in a control sample (e.g., sample from a healthy subject not afflicted with cancer) and preferably, the average expression level or copy number of the biomarker or MCR in several control samples.

“Methylation status” of a biomarker refers to the methylation pattern, e.g., methylation of the promoter of the biomarker, and/or methylation levels of the biomarker. DNA methylation is a heritable, reversible and epigenetic change. Yet, DNA methylation has the potential to alter gene expression, which has developmental and genetic consequences. DNA methylation has been linked to cancer, as described in, for example, Laird, et al. (1994) Human Molecular Genetics 3:1487-1495 and Laird, P. (2003) Nature 3:253-266, the contents of which are incorporated herein by reference. For example, methylation of CpG oligonucleotides in the promoters of tumor suppressor genes can lead to their inactivation. In addition, alterations in the normal methylation process are associated with genomic instability (Lengauer. et al. Proc. Natl. Acad. Sci. USA 94:2545-2550, 1997). Such abnormal epigenetic changes may be found in many types of cancer and can, therefore, serve as potential biomarkers for oncogenic transformation.

Methods for determining methylation include restriction landmark genomic scanning (Kawai, et al., Mol. Cell. Biol. 14:7421-7427, 1994), methylation-sensitive arbitrarily primed PCR (Gonzalgo, et al., Cancer Res. 57:594-599, 1997); digestion of genomic DNA with methylation-sensitive restriction enzymes followed by Southern analysis of the regions of interest (digestion-Southern method); PCR-based process that involves digestion of genomic DNA with methylation-sensitive restriction enzymes prior to PCR amplification (Singer-Sam, et al., Nucl. Acids Res. 18:687, 1990); genomic sequencing using bisulfite treatment (Frommer, et al., Proc. Natl. Acad. Sci. USA 89:1827-1831, 1992); methylation-specific PCR (MSP) (Herman, et al. Proc. Natl. Acad. Sci. USA 93:9821-9826, 1992); and restriction enzyme digestion of PCR products amplified from bisulfite-converted DNA (Sadri and Hornsby, Nucl. Acids Res. 24:5058-5059, 1996; and Xiong and Laird, Nucl. Acids. Res. 25:2532-2534, 1997); PCR techniques for detection of gene mutations (Kuppuswamy, et al., Proc. Natl. Acad. Sci. USA 88:1143-1147, 1991) and quantitation of allelic-specific expression (Szabo and Mann, Genes Dev. 9:3097-3108, 1995; and Singer-Sam, et al., PCR Methods Appl. 1:160-163, 1992); and methods described in U.S. Pat. No. 6,251,594, the contents of which are incorporated herein by reference. An integrated genomic and epigenomic analysis as described in Zardo, et al. (2000) Nature Genetics 32:453-458, may also be used.

The term “altered activity” of a biomarker refers to an activity of a biomarker which is increased or decreased in a disease state, e.g., in a cancer sample, as compared to the activity of the biomarker in a normal, control sample. Altered activity of a biomarker may be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors, or altered methylation status.

The term “altered structure” of a biomarker refers to the presence of mutations or allelic variants within the biomarker gene or maker protein, e.g., mutations which affect expression or activity of the biomarker, as compared to the normal or wild-type gene or protein. For example, mutations include, but are not limited to substitutions, deletions, or addition mutations. Mutations may be present in the coding or non-coding region of the biomarker.

A “biomarker nucleic acid” is a nucleic acid (e.g., DNA, mRNA, cDNA) encoded by or corresponding to a biomarker of the invention. The biomarker nucleic acid molecules also include RNA comprising the entire or a partial sequence of any of the nucleic acid sequences or the complement of such a sequence, wherein all thymidine residues are replaced with uridine residues. A “biomarker protein” is a protein encoded by or corresponding to a biomarker of the invention. A biomarker protein comprises the entire or a partial sequence of a protein encoded by any of the sequences or a fragment thereof. The terms “protein” and “polypeptide” are used interchangeably herein.

A “biomarker,” as used herein, includes any nucleic acid sequence present in an MCR as set forth in Table 3, or a protein encoded by such a sequence.

Biomarkers identified herein include diagnostic and therapeutic biomarkers. A single biomarker may be a diagnostic biomarker, a therapeutic biomarker, or both a diagnostic and therapeutic biomarker.

As used herein, the term “therapeutic biomarker” includes biomarkers which are believed to be involved in the development (including maintenance, progression, angiogenesis, and/or metastasis) of cancer. The cancer-related functions of a therapeutic biomarker may be confirmed by, e.g., (1) increased or decreased copy number (by, e.g., fluorescence in situ hybridization (FISH), and FISH plus spectral karotype (SKY), or quantitative PCR (qPCR)) or mutation (e.g., by sequencing), overexpression or underexpression (e.g., by in situ hybridization (ISH), Northern Blot, or qPCR), increased or decreased protein levels (e.g., by immunohistochemistry (IHC)), or increased or decreased protein activity (determined by, for example, modulation of a pathway in which the biomarker is involved), e.g., in more than about 5%, 6%, 7%, 8%, 9%, 10%, 11%, 12%, 13%, 14%, 15%, 20%, 25%, or more of human cancers; (2) the inhibition of cancer cell proliferation and growth, e.g., in soft agar, by, e.g., RNA interference (“RNAi”) of the biomarker; (3) the ability of the biomarker to enhance transformation of mouse embryo fibroblasts (MEFs) by oncogenes, e.g., Myc and RAS, or by RAS alone; (4) the ability of the biomarker to enhance or decrease the growth of tumor cell lines, e.g., in soft agar; (5) the ability of the biomarker to transform primary mouse cells in SCID explant; and/or; (6) the prevention of maintenance or formation of tumors, e.g., tumors arising de novo in an animal or tumors derived from human cancer cell lines, by inhibiting or activating the biomarker. In one embodiment, a therapeutic biomarker may be used as a diagnostic biomarker.

As used herein, the term “diagnostic biomarker” includes biomarkers which are useful in the diagnosis of cancer, e.g., over- or under-activity emergence, expression, growth, remission, recurrence or resistance of tumors before, during or after therapy. The predictive functions of the biomarker may be confirmed by, e.g., (1) increased or decreased copy number (e.g., by FISH, FISH plus SKY, or qPCR), overexpression or underexpression (e.g., by ISH, Northern Blot, or qPCR), increased or decreased protein level (e.g., by IHC), or increased or decreased activity (determined by, for example, modulation of a pathway in which the biomarker is involved), e.g., in more than about 5%, 6%, 7%, 8%, 9%, 10%, 11%, 12%, 13%, 14%, 15%, 20%, 25%, or more of human cancers; (2) its presence or absence in a biological sample, e.g., a sample containing tissue, whole blood, serum, plasma, buccal scrape, saliva, cerebrospinal fluid, urine, stool, stool, bile, pancreatic cells or pancreatic tissue tissue from a subject, e.g. a human, afflicted with cancer; (3) its presence or absence in clinical subset of subjects with cancer (e.g., those responding to a particular therapy or those developing resistance).

Diagnostic biomarkers also include “surrogate biomarkers,” e.g., biomarkers which are indirect biomarkers of cancer progression.

The term “probe” refers to any molecule which is capable of selectively binding to a specifically intended target molecule, for example a biomarker of the invention. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic monomers.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a spatially or temporally restricted manner.

An “RNA interfering agent” as used herein, is defined as any agent which interferes with or inhibits expression of a target gene, e.g., a biomarker of the invention, by RNA interference (RNAi). Such RNA interfering agents include, but are not limited to, nucleic acid molecules including RNA molecules which are homologous to the target gene, e.g., a biomarker of the invention, or a fragment thereof, short interfering RNA (siRNA), and small molecules which interfere with or inhibit expression of a target gene by RNA interference (RNAi).

“RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” or “inhibition of biomarker gene expression” includes any decrease in expression or protein activity or level of the target gene (e.g., a biomarker gene of the invention) or protein encoded by the target gene, e.g., a biomarker protein of the invention. The decrease may be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the over hang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated be reference herein).

RNA interfering agents, e.g., siRNA molecules, may be administered to a subject having or at risk for having cancer, to inhibit expression of a biomarker gene of the invention, e.g., a biomarker gene which is overexpressed in cancer (such as the biomarkers listed in Table 2) and thereby treat, prevent, or inhibit cancer in the subject.

A “constitutive” promoter is a nucleotide sequence which, when operably linked with a polynucleotide which encodes or specifies a gene product, causes the gene product to be produced in a living human cell under most or all physiological conditions of the cell.

An “inducible” promoter is a nucleotide sequence which, when operably linked with a polynucleotide which encodes or specifies a gene product, causes the gene product to be produced in a living human cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living human cell substantially only if the cell is a cell of the tissue type corresponding to the promoter.

A “transcribed polynucleotide” is a polynucleotide (e.g. an RNA, a cDNA, or an analog of one of an RNA or cDNA) which is complementary to or homologous with all or a portion of a mature RNA made by transcription of a biomarker of the invention and normal post-transcriptional processing (e.g. splicing), if any, of the transcript, and reverse transcription of the transcript.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The terms “homology” or “identity,” as used interchangeably herein, refer to sequence similarity between two polynucleotide sequences or between two polypeptide sequences, with identity being a more strict comparison. The phrases “percent identity or homology” and “% identity or homology” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value there between. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences. The term “substantial homology,” as used herein, refers to homology of at least 50%, more preferably, 60%, 70%, 80%, 90%, 95% or more.

A biomarker is “fixed” to a substrate if it is covalently or non-covalently associated with the substrate such the substrate can be rinsed with a fluid (e.g. standard saline citrate, pH 7.4) without a substantial fraction of the biomarker dissociating from the substrate.

As used herein, a “naturally-occurring” nucleic acid molecule refers to an RNA or DNA molecule having a nucleotide sequence that occurs in nature (e.g. encodes a natural protein).

Cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

A kit is any manufacture (e.g. a package or container) comprising at least one reagent, e.g. a probe, for specifically detecting a biomarker of the invention, the manufacture being promoted, distributed, or sold as a unit for performing the methods of the present invention.

### II. USES OF THE INVENTION

The present invention is based, at least in part, on the generation of non-human animal models of pancreatic ductal adenocarcinoma which recapitulate the genetic and histological features of the human disease, including the initiation, maintenance, and progression of the disease. Accordingly, the present invention provides methods for identifying compounds that modulate, e.g., inhibit, treat, or prevent pancreatic adenocarcinoma using the animal models described herein. The present invention also provides methods for identifying pancreatic cancer specific biomarkers which are capable of use in diagnosis or prognosis of pancreatic cancer. These biomarkers serve as diagnostics for detection of early stage disease in, e.g., asymptomatic subjects, or to identify stage or progression of pancreatic cancer in a subject.

As described herein, misexpression, e.g., activation of Kras and misexpression, e.g., decreased expression, of one or more tumor suppressor genes, e.g., Ink4a/Arf, Ink4a, Arf, p53, Smad4/Dpc, Lkb1, Brca2, or Mlh1 in an animal model of the invention results in the development and progression of pancreatic cancer, e.g., pancreatic adenocarcinoma that mimics the initiation, progression and/or maintenance of the disease in humans. Thus, animal models as described herein, as well as specific cell types, e.g., pancreatic, stomal, acinar, ductal, purified cells derived from a pancreatic adenocarcinoma animal model, or cell lines generated from these cell types and derived from a pancreatic adenocarcinoma animal model, as described herein, can be used in screening assays to identify agents that modulate, treat, prevent, or diagnose pancreatic adenocarcinoma.

A. Biomarker Discovery

The present invention provides methods for the identification of diagnostic, prognostic and/or pharmacogenomic biomarkers for e.g., the initiation, progression and/or maintenance, of pancreatic cancer, e.g., pancreatic adenocarcinoma or prognostic biomarkers. The inbred, genetically determined animal models of the present invention are maintained under controlled environmental conditions and show highly reproducible and rapid pancreatic adenocarcinoma development making them ideal tools for identification of stage-specific biomarkers for the disease including, but not limited to, early stage, advanced stage, and late stage disease biomarkers. Also, the animal models of the invention allow the identification of biomarkers that are specific for particular genetic lesions, including loss of function of specific genes including, but not limited to, Ink4a/Arf, Ink4a, Arf, p53, Smad4/Dpc, Lkb1, Brca2, or Mlh1.

In general, a method of identifying biomarkers associated with pancreatic cancer, e.g., pancreatic adenocarcinoma involves comparing the amount and/or activity of a biomarker in a sample, e.g., a sample containing tissue, whole blood, serum, plasma, buccal scrape, saliva, urine, stool, bile, pancreatic cells or pancreatic tissue, from an animal model as described herein, e.g., an animal model carrying an activating mutation of KRAS in addition to one or more misexpressed tumor suppressor genes or loci, from a wild-type animal, e.g., a control animal. Differences between the animals in the amount and/or activity of a biomarker indicates that biomarker is associated with pancreatic adenocarcinoma.

In one embodiment, a sample, e.g., a sample containing tissue, whole blood, serum, plasma, buccal scrape, saliva, urine, stool, bile, pancreatic cells or pancreatic tissue, from an animal model of the invention is compared to a sample, e.g., a sample containing tissue, whole blood, serum, plasma, buccal scrape, saliva, urine, stool, bile, pancreatic cells or pancreatic tissue, from a wild-type animal, e.g., a control animal and used to identify biomarkers.

The animal models of the invention and cell lines isolated therefrom may also be used for the discovery of diagnostics which react with the stromal component, such as specific tumor associated proteases such as cathepsins that can serve as suitable substrates for fluorescent imaging probes (Ntziachristos, V., et al. (2003) Eur Radiol 13, 195-208; Ntziachristos, V., et al. (2002) Nat Med 8, 757-760; Weissleder, R., and Ntziachristos, V. (2003) Nat Med 9, 123-128). The characterization of the stroma in pancreatic adenocarcinoma is vital in diagnostics given its important contribution to tumor size. In vitro, culturing techniques enable the cultivation of both tumor cells and associated stroma allowing the potential to study expression profiles of cell surface markers (e.g., using phage-display techniques (Spear, M. A., et al (2001) Cancer Gene Ther 8, 506-511) in each tumor compartment. Cell lines have been used for subcutaneous injection of SCID mice, and have shown robust growth and retention of the origin tumor cell morphology and genetics.

Accordingly, in another embodiment, tissue from the stroma is compared to the tissue from the epithelial compartment of the tumor and used to identify biomarkers specific to the stromal compartment or the epithelial compartment of the tumor. In a further embodiment, cell lines are generated from the pancreatic tumor in its entirety and utilized to identify biomarkers as described above. In yet another embodiment, cell lines are generated from the stromal and/or epithelial component of the pancreatic tumor in its entirety and utilized to identify biomarkers as described above. In one embodiment, cells from the stromal component of the tumor are mixed with cells from the epithelial component of the tumor.

In one embodiment, a biomarker is identified based on the patterns of accumulation of a variety of molecules that may regulate, for example, growth of a tumor that are surveyed using immunohistochemical methods known in the art and as described herein. Screens directed at analyzing expression of specific genes or groups of molecules implicated in pathogenesis can be continued during the life of the animal model. Expression can be monitored by immunohistochemistry as well as by protein and RNA blotting techniques. Metastatic foci, once formed, can also be subjected to such comparative surveys. T his analysis can also be extended to include an assessment of the effects of various treatment paradigms (including the use of compounds identified as modulating pancreatic adenocarcinoma identified as described herein in the animal models of the invention) on differential gene expression. The information derived from the surveys of differential gene expression can ultimately be correlated with disease initiation and progression in the animal model.

In one embodiment, a biomarker is a protein or fragment thereof. As an exemplary embodiment, a protein and/or fragment of a biomarker of the invention is identified from the blood (whole blood, serum, and/or plasma) buccal scrape, saliva, urine, stool, bile, pancreatic cells or pancreatic tissue, of the animal models of the invention based on its misexpression when compared to control animals. The serum specimens are analyzed by protein discovery platforms known in the art (see Example 2) that resolve the specimens into fractions that can readily be subjected to analysis for identification of specific peptides, e.g., by mass spectrometry, to allow the identification of stage-specific biomarkers that are diagnostic, prognostic and/or pharmacogenomic for e.g., initiation, progression and/or maintenance, of pancreatic adenocarcinoma.

In one embodiment, antibodies, e.g., monoclonal and/or polyclonal antibodies, are raised against these biomarkers. Epitopes for antibody generation can be chosen by those skilled in the art to recognize the orthologous protein in humans. These antibodies can be evaluated for their applicability as human diagnostic, prognostic and/or pharmacogenomic biomarkers using, for example, ELISA-based assays, to test samples, e.g., serum, from human pancreatic adenocarcinoma subjects and from control subjects in prospective clinical studies whose clinical follow-up reveals the development of pancreatic cancer, e.g., pancreatic adenocarcinoma.

In another embodiment, protein biomarkers are identified utilizing specific protein chips (such as the Zyomyx cytokine chip).

In another embodiment, a biomarker is a nucleic acid or fragment thereof. Nucleic acid biomarkers can be identified based on, for example, gene expression patterns comparing the expression pattern of animal models of the invention with control littermates. For example, the expression pattern of one or more genes may form part of a “gene expression profile” or “transcriptional profile” which may be then be used in such an assessment. “Gene expression profile” or “transcriptional profile”, as used herein, includes the pattern of mRNA expression obtained for a given tissue or cell type under a given set of conditions. Such conditions may include, but are not limited to, cell growth, proliferation, differentiation, transformation, tumorigenesis, metastasis, and carcinogen exposure.

Gene expression profiles may be generated, for example, by utilizing a differential gene expression procedure, Northern analysis and/or RT-PCR. Gene expression profiles may be characterized for known states within the cell- and/or animal-based model systems. Subsequently, these known gene expression profiles may be compared to ascertain the effect a test compound has to modify such gene expression profiles, and to cause the profile to more closely resemble that of a more desirable profile.

In another embodiment, non-invasive imaging techniques, e.g., magnetic resonance imaging (MRI) are utilized to monitor the development and growth of pancreatic tumors in the model system of the present invention in order to permit correlation of cancer progression with the biomarkers discovered as described herein.

In another aspect of the invention, biomarkers are identified within chromosomal regions (MCRs) which are structurally altered leading to a different copy number in cancer cells, e.g., cells from the animal models of pancreatic cancer described herein, as compared to normal (i.e. non-cancerous) cells. Accordingly, the present invention is based, in part, on the identification of chromosomal regions (MCRs) which are structurally altered leading to a different copy number in cancer cells as compared to normal (i.e. non-cancerous) cells. Furthermore, the present invention is based, in part, on the identification of biomarkers, e.g., biomarkers which reside in the MCRs of the invention, which have an altered amount, structure, and/or activity in cancer cells as compared to normal (i.e., non-cancerous) cells. The biomarkers of the invention correspond to DNA, cDNA, RNA, and polypeptide molecules which can be detected in one or both of normal and cancerous cells.

The presence, absence, and/or copy number of one or more of the MCRs of the invention in a sample is also correlated with the cancerous state of the tissue. The invention thus provides compositions, kits, and methods for assessing the cancerous state of cells (e.g. cells obtained from a non-human, cultured non-human cells, and in vivo cells) as well as methods for treatment, prevention, and/or inhibition of cancer using a modulator, e.g., an agonist or antagonist, of a biomarker of the invention.

B. Screening Assays

In one aspect, the invention provides screening methods (also referred to herein as a “screening assays”) for identifying modulators, i.e., candidate or test compounds or agents (e.g., proteins, peptides, peptidomimetics, small molecules (organic or inorganic) or other drugs) which modulate, treat, or prevent pancreatic cancer or modulate a molecule involved in the initiation, maintenance, and/or progression of pancreatic cancer, e.g. pancreatic adenocarcinoma.

In one embodiment, cells derived from an animal model of the invention, e.g., cells which misexpress one or more tumor suppressor genes and which have an activating Kras mutation, can be contacted ex vivo with one or more test compound and a biological response regulated by Ink4a/Arf, p53, or a molecule in a signal transduction pathway involving one or more tumor suppressor genes or other genes associated with pancreatic cancer can be monitored. Modulation of the response in cells or a molecule in a signal transduction pathway involving one or more tumor suppressor genes or other genes associated with pancreatic cancer (as compared to an appropriate control such as, for example, untreated cells or cells treated with a control agent) identifies a test compound as a modulator of pancreatic cancer, e.g. pancreatic adenocarcinoma.

In another embodiment, one or more test compound is administered directly to an animal model of the invention in vivo (e.g., an animal model of pancreatic cancer as described herein), to identify a test compound that modulates the in vivo responses of cells which misexpress one or more tumor suppressor genes and have an activating Kras mutation or to evaluate the effect of the test compound on the initiation, maintenance, and/or progression of pancreatic cancer in the animal or on the symptoms of the disease. The response of the animals to the exposure may be monitored by assessing the reversal of pancreatic cancer, or symptoms associated therewith, for example, reduction in tumor burden, tumor size, and invasive and/or metastatic potential before and after treatment.

The test compound can be administered to an animal model as a pharmaceutical composition. Such compositions typically comprise the test compound and a pharmaceutically acceptable carrier. As used herein the term “pharmaceutically acceptable carrier” includes any and all solvents, dispersion media, coatings, antibacterial and antifungal compounds, isotonic and absorption delaying compounds, and the like, compatible with pharmaceutical administration. The use of such media and compounds for pharmaceutically active substances is well known in the art. Except insofar as any conventional media or compound is incompatible with the active compound, use thereof in the compositions is contemplated. Supplementary active compounds can also be incorporated into the compositions.

For practicing the screening methods ex vivo, cells derived from the animal models of the invention can be isolated from an animal or embryo by standard methods and incubated (i.e., cultured) in vitro with a test compound. Cells (e.g., pancreatic cells, e.g., pancreatic epithelial, stomal, acinar, ductal) can be isolated from animal models of the invention by standard techniques.

Following contact of the cells with one or more test compound (either ex vivo or in vivo), the effect of the test compound on the biological response of the cells can be determined by any one of a variety of suitable methods including, e.g., light microscopic analysis of the cells, histochemical analysis of the cells, production of proteins, induction of certain genes, e.g., tumor suppressor genes.

The invention also provides methods for identifying modulators, i.e., candidate or test compounds or agents which (a) bind to a biomarker of the invention, or (b) have a modulatory (e.g., stimulatory or inhibitory) effect on the activity of a biomarker of the invention or, more specifically, (c) have a modulatory effect on the interactions of a biomarker of the invention with one or more of its natural substrates (e.g., peptide, protein, hormone, co-factor, or nucleic acid), or (d) have a modulatory effect on the expression of a biomarker of the invention. Such assays typically comprise a reaction between the biomarker and one or more assay components. The other components may be either the test compound itself, or a combination of test compound and a natural binding partner of the biomarker. Compounds identified via assays such as those described herein may be useful, for example, for modulating, e.g., inhibiting, ameliorating, treating, or preventing cancer.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233. Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

In one embodiment, the invention provides assays for screening candidate or test compounds which are substrates of a biomarker of the invention or biologically active portion thereof. In another embodiment, the invention provides assays for screening candidate or test compounds which bind to a biomarker of the invention or biologically active portion thereof. Determining the ability of the test compound to directly bind to a biomarker can be accomplished, for example, by coupling the compound with a radioisotope or enzymatic label such that binding of the compound to the biomarker can be determined by detecting the labeled biomarker compound in a complex. For example, compounds (e.g., biomarker substrates) can be labeled with ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

In another embodiment, the invention provides assays for screening candidate or test compounds which modulate the activity of a biomarker of the invention or a biologically active portion thereof. In all likelihood, the biomarker can, in vivo, interact with one or more molecules, such as, but not limited to, peptides, proteins, hormones, cofactors and nucleic acids. For the purposes of this discussion, such cellular and extracellular molecules are referred to herein as “binding partners” or biomarker “substrate”.

One necessary embodiment of the invention in order to facilitate such screening is the use of the biomarker to identify its natural in vivo binding partners. There are many ways to accomplish this which are known to one skilled in the art. One example is the use of the biomarker protein as “bait protein” in a two-hybrid assay or three-hybrid assay (see, e.g., U.S. Pat. No. 5,283,317; Zervos et al, 1993, Cell 72:223-232; Madura et al, 1993, J. Biol. Chem. 268:12046-12054; Bartel et al, 1993, Biotechniques 14:920-924; Iwabuchi et al, 1993 Oncogene 8:1693-1696; Brent WO94/10300) in order to identify other proteins which bind to or interact with the biomarker (binding partners) and, therefore, are possibly involved in the natural function of the biomarker. Such biomarker binding partners are also likely to be involved in the propagation of signals by the biomarker or downstream elements of a biomarker-mediated signaling pathway. Alternatively, such biomarker binding partners may also be found to be inhibitors of the biomarker.

The two-hybrid system is based on the modular nature of most transcription factors, which consist of separable DNA-binding and activation domains. Briefly, the assay utilizes two different DNA constructs. In one construct, the gene that encodes a biomarker protein fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a biomarker-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be readily detected and cell colonies containing the functional transcription factor can be isolated and used to obtain the cloned gene which encodes the protein which interacts with the biomarker protein.

In a further embodiment, assays may be devised through the use of the invention for the purpose of identifying compounds which modulate (e.g., affect either positively or negatively) interactions between a biomarker and its substrates and/or binding partners. Such compounds can include, but are not limited to, molecules such as antibodies, peptides, hormones, oligonucleotides, nucleic acids, and analogs thereof. Such compounds may also be obtained from any available source, including systematic libraries of natural and/or synthetic compounds. The preferred assay components for use in this embodiment is a cancer biomarker identified herein, the known binding partner and/or substrate of same, and the test compound. Test compounds can be supplied from any source.

The basic principle of the assay systems used to identify compounds that interfere with the interaction between the biomarker and its binding partner involves preparing a reaction mixture containing the biomarker and its binding partner under conditions and for a time sufficient to allow the two products to interact and bind, thus forming a complex. In order to test an agent for inhibitory activity, the reaction mixture is prepared in the presence and absence of the test compound. The test compound can be initially included in the reaction mixture, or can be added at a time subsequent to the addition of the biomarker and its binding partner. Control reaction mixtures are incubated without the test compound or with a placebo. The formation of any complexes between the biomarker and its binding partner is then detected. The formation of a complex in the control reaction, but less or no such formation in the reaction mixture containing the test compound, indicates that the compound interferes with the interaction of the biomarker and its binding partner. Conversely, the formation of more complex in the presence of compound than in the control reaction indicates that the compound may enhance interaction of the biomarker and its binding partner.

The assay for compounds that interfere with the interaction of the biomarker with its binding partner may be conducted in a heterogeneous or homogeneous format. Heterogeneous assays involve anchoring either the biomarker or its binding partner onto a solid phase and detecting complexes anchored to the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the biomarkers and the binding partners (e.g., by competition) can be identified by conducting the reaction in the presence of the test substance, i.e., by adding the test substance to the reaction mixture prior to or simultaneously with the biomarker and its interactive binding partner. Alternatively, test compounds that disrupt preformed complexes, e.g., compounds with higher binding constants that displace one of the components from the complex, can be tested by adding the test compound to the reaction mixture after complexes have been formed.

The various formats are briefly described below.

In a heterogeneous assay system, either the biomarker or its binding partner is anchored onto a solid surface or matrix, while the other corresponding non-anchored component may be labeled, either directly or indirectly. In practice, microtitre plates are often utilized for this approach. The anchored species can be immobilized by a number of methods, either non-covalent or covalent, that are typically well known to one who practices the art. Non-covalent attachment can often be accomplished simply by coating the solid surface with a solution of the biomarker or its binding partner and drying. Alternatively, an immobilized antibody specific for the assay component to be anchored can be used for this purpose. Such surfaces can often be prepared in advance and stored.

In related embodiments, a fusion protein can be provided which adds a domain that allows one or both of the assay components to be anchored to a matrix. For example, glutathione-S-transferase/biomarker fusion proteins or glutathione-S-transferase/binding partner can be adsorbed onto glutathione sepharose beads (Sigma Chemical, St. Louis, Mo.) or glutathione derivatized microtiter plates, which are then combined with the test compound or the test compound and either the non-adsorbed biomarker or its binding partner, and the mixture incubated under conditions conducive to complex formation (e.g., physiological conditions). Following incubation, the beads or microtiter plate wells are washed to remove any unbound assay components, the immobilized complex assessed either directly or indirectly, for example, as described above. Alternatively, the complexes can be dissociated from the matrix, and the level of biomarker binding or activity determined using standard techniques.

Other techniques for immobilizing proteins on matrices can also be used in the screening assays of the invention. For example, either a biomarker or a biomarker binding partner can be immobilized utilizing conjugation of biotin and streptavidin. Biotinylated biomarker protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the protein-immobilized surfaces can be prepared in advance and stored.

In order to conduct the assay, the corresponding partner of the immobilized assay component is exposed to the coated surface with or without the test compound. After the reaction is complete, unreacted assay components are removed (e.g., by washing) and any complexes formed will remain immobilized on the solid surface. The detection of complexes anchored on the solid surface can be accomplished in a number of ways. Where the non-immobilized component is pre-labeled, the detection of label immobilized on the surface indicates that complexes were formed. Where the non-immobilized component is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds which modulate (inhibit or enhance) complex formation or which disrupt preformed complexes can be detected.

In an alternate embodiment of the invention, a homogeneous assay may be used. This is typically a reaction, analogous to those mentioned above, which is conducted in a liquid phase in the presence or absence of the test compound. The formed complexes are then separated from unreacted components, and the amount of complex formed is determined. As mentioned for heterogeneous assay systems, the order of addition of reactants to the liquid phase can yield information about which test compounds modulate (inhibit or enhance) complex formation and which disrupt preformed complexes.

In such a homogeneous assay, the reaction products may be separated from unreacted assay components by any of a number of standard techniques, including but not limited to: differential centrifugation, chromatography, electrophoresis and immunoprecipitation. In differential centrifugation, complexes of molecules may be separated from uncomplexed molecules through a series of centrifugal steps, due to the different sedimentation equilibria of complexes based on their different sizes and densities (see, for example, Rivas, G., and Minton, A. P., Trends Biochem Sci 1993 August; 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the complex as compared to the uncomplexed molecules may be exploited to differentially separate the complex from the remaining individual reactants, for example through the use of ion-exchange chromatography resins. Such resins and chromatographic techniques are well known to one skilled in the art (see, e.g., Heegaard, 1998, J Mol. Recognit. 11:141-148; Hage and Tweed, 1997, J. Chromatogr. B. Biomed. Sci. Appl., 699:499-525). Gel electrophoresis may also be employed to separate complexed molecules from unbound species (see, e.g., Ausubel et al (eds.), In: Current Protocols in Molecular Biology, J. Wiley & Sons, New York. 1999). In this technique, protein or nucleic acid complexes are separated based on size or charge, for example. In order to maintain the binding interaction during the electrophoretic process, nondenaturing gels in the absence of reducing agent are typically preferred, but conditions appropriate to the particular interactants will be well known to one skilled in the art. Immunoprecipitation is another common technique utilized for the isolation of a protein-protein complex from solution (see, e.g., Ausubel et al (eds.), In: Current Protocols in Molecular Biology, J. Wiley & Sons, New York. 1999). In this technique, all proteins binding to an antibody specific to one of the binding molecules are precipitated from solution by conjugating the antibody to a polymer bead that may be readily collected by centrifugation. The bound assay components are released from the beads (through a specific proteolysis event or other technique well known in the art which will not disturb the protein-protein interaction in the complex), and a second immunoprecipitation step is performed, this time utilizing antibodies specific for the correspondingly different interacting assay component. In this manner, only formed complexes should remain attached to the beads. Variations in complex formation in both the presence and the absence of a test compound can be compared, thus offering information about the ability of the compound to modulate interactions between the biomarker and its binding partner.

Also within the scope of the present invention are methods for direct detection of interactions between the biomarker and its natural binding partner and/or a test compound in a homogeneous or heterogeneous assay system without further sample manipulation. For example, the technique of fluorescence energy transfer may be utilized (see, e.g., Lakowicz et al, U.S. Pat. No. 5,631,169; Stavrianopoulos et al, U.S. Pat. No. 4,868,103). Generally, this technique involves the addition of a fluorophore label on a first ‘donor’ molecule (e.g., biomarker or test compound) such that its emitted fluorescent energy will be absorbed by a fluorescent label on a second, ‘acceptor’ molecule (e.g., biomarker or test compound), which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter). A test substance that either enhances or hinders participation of one of the species in the preformed complex will result in the generation of a signal variant to that of background. In this way, test substances that modulate interactions between a biomarker and its binding partner can be identified in controlled assays.

In another embodiment, modulators of biomarker expression are identified in a method wherein a cell is contacted with a candidate compound and the expression of mRNA or protein, corresponding to a biomarker in the cell, is determined. The level of expression of mRNA or protein in the presence of the candidate compound is compared to the level of expression of mRNA or protein in the absence of the candidate compound. The candidate compound can then be identified as a modulator of biomarker expression based on this comparison. For example, when expression of biomarker mRNA or protein is greater (statistically significantly greater) in the presence of the candidate compound than in its absence, the candidate compound is identified as a stimulator of biomarker mRNA or protein expression. Conversely, when expression of biomarker mRNA or protein is less (statistically significantly less) in the presence of the candidate compound than in its absence, the candidate compound is identified as an inhibitor of biomarker mRNA or protein expression. The level of biomarker mRNA or protein expression in the cells can be determined by methods described herein for detecting biomarker mRNA or protein.

In another aspect, the invention pertains to a combination of two or more of the assays described herein. For example, a modulating agent can be identified using a cell-based or a cell free assay, and the ability of the agent to modulate the activity of a biomarker protein can be further confirmed in vivo, e.g., in a whole animal model for cancer, cellular transformation and/or tumorigenesis, such as those described herein. Additional animal based models of cancer are well known in the art (reviewed in Animal Models of Cancer Predisposition Syndromes, Hiai, H and Hino, O (eds.) 1999, Progress in Experimental Tumor Research, Vol. 35; Clarke A R Carcinogenesis (2000) 21:435-41) and include, for example, carcinogen-induced tumors (Rithidech, K et al. Mutat Res (1999) 428:33-39; Miller, M L et al. Environ Mol Mutagen (2000) 35:319-327), injection and/or transplantation of tumor cells into an animal, as well as animals bearing mutations in growth regulatory genes, for example, oncogenes (e.g., ras) (Arbeit, J M et al. Am J Pathol (1993) 142:1187-1197; Sinn, E et al. Cell (1987) 49:465-475; Thorgeirsson, S S et al. Toxicol Lett (2000) 112-113:553-555) and tumor suppressor genes (e.g., p53) (Vooijs, M et al. Oncogene (1999) 18:5293-5303; Clark A R Cancer Metast Rev (1995) 14:125-148; Kumar, T R et al. J Intern Med (1995) 238:233-238; Donehower, L A et al. (1992) Nature 356215-221). Furthermore, experimental model systems are available for the study of, for example, ovarian cancer (Hamilton, T C et al. Semin Oncol (1984) 11:285-298; Rahman, N A et al. Mol Cell Endocrinol (1998) 145:167-174; Beamer, W G et al. Toxicol Pathol (1998) 26:704-710), gastric cancer (Thompson, J et al. Int J Cancer (2000) 86:863-869; Fodde, R et al. Cytogenet Cell Genet (1999) 86:105-111), breast cancer (Li, M et al. Oncogene (2000) 19:1010-1019; Green, J E et al. Oncogene (2000) 19:1020-1027), melanoma (Satyamoorthy, K et al. Cancer Metast Rev (1999) 18:401-405), and prostate cancer (Shirai, T et al. Mutat Res (2000) 462:219-226; Bostwick, D G et al. Prostate (2000) 43:286-294). Animal models described in, for example, Chin L. et al (1999) Nature 400(6743):468-72, may also be used in the methods of the invention.

This invention further pertains to novel agents identified by the above-described screening assays. Accordingly, it is within the scope of this invention to further use an agent identified as described herein in an appropriate animal model. For example, an agent identified as described herein (e.g., a biomarker modulating agent, a small molecule, an antisense biomarker nucleic acid molecule, a ribozyme, a biomarker-specific antibody, or fragment thereof, a biomarker protein, a biomarker nucleic acid molecule, an RNA interfering agent, e.g., an siRNA molecule targeting a biomarker of the invention, or a biomarker-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein. In one embodiment, the invention features a method of treating a subject having pancreatic cancer that involves administering to the subject a compound identified as a modulator of pancreatic cancer, e.g., pancreatic adenocarcinoma, such that treatment occurs.

### III. METHODS OF USE

The compositions, kits, and methods of the invention have the following uses, among others:
1) assessing whether a subject is afflicted with cancer, e.g., pancreatic adenocarcinoma;
2) assessing the stage of cancer, e.g., pancreatic adenocarcinoma, in a human subject;
3) assessing the grade of cancer, e.g., pancreatic adenocarcinoma, in a subject,
4) assessing the benign or malignant nature of cancer, e.g., pancreatic adenocarcinoma, in a subject;
5) assessing the metastatic potential of cancer, e.g., pancreatic adenocarcinoma, in a subject;
6) assessing the histological type of neoplasm associated with cancer, e.g., pancreatic adenocarcinoma, in a subject;
7) making antibodies, antibody fragments or antibody derivatives that are useful for treating cancer, e.g., pancreatic adenocarcinoma, and/or assessing whether a subject is afflicted with cancer;
8) assessing the presence of cancer, e.g., pancreatic adenocarcinoma, cells;
9) assessing the efficacy of one or more test compounds for inhibiting cancer, e.g., pancreatic adenocarcinoma, in a subject;
10) assessing the efficacy of a therapy for inhibiting cancer, e.g., pancreatic adenocarcinoma, in a subject;
11) monitoring the progression of cancer, e.g., pancreatic adenocarcinoma, in a subject;
12) selecting a composition or therapy for inhibiting cancer, e.g., pancreatic adenocarcinoma in a subject;
13) treating a subject afflicted with cancer, e.g., pancreatic adenocarcinoma;
14) inhibiting cancer, e.g., pancreatic adenocarcinoma in a subject;
15) assessing the carcinogenic potential of a test compound;
16) preventing the onset of cancer, e.g., pancreatic adenocarcinoma in a subject at risk for developing cancer;
17) assessing for the presence of specific mutations or activation of specific signaling pathways in pancreatic adenocarcinomas; and
18) determining the impact of therapeutics targeted to specific signaling pathways in pancreatic adenocarcinomas.

The present invention provides methods to identify biomarkers which are modulated in pancreatic cancer cells as compared to their amount and/or activity of a biomarker in normal (i.e. non-cancerous) pancreatic cells. The modulated amount and/or activity of one or more of these biomarkers in a sample, e.g., a sample containing blood, e.g., serum, urine, stool, bile, pancreatic cells or pancreatic juices, is herein correlated with the cancerous state of the tissue. The invention provides compositions, kits, and methods for assessing the cancerous state of pancreatic cells (e.g., cells obtained from a non-human, cultured non-human cells, and in vivo cells) as well as treating subjects afflicted with pancreatic adenocarcinoma.

The invention thus includes a method of assessing whether a subject is afflicted with cancer or is at risk for developing cancer. This method comprises comparing the amount, structure, and/or activity, e.g., the presence, absence, copy number, expression level, protein level, protein activity, presence of mutations, e.g., mutations which affect activity of the biomarker (e.g., substitution, deletion, or addition mutations), and/or methylation status, of a biomarker in a subject sample with the normal level. A significant difference between the amount, structure, or activity of the biomarker in the subject sample and the normal level is an indication that the subject is afflicted with cancer. The invention also provides a method for assessing whether a subject is afflicted with cancer or is at risk for developing cancer by comparing the level of expression of biomarker(s) within an MCR or copy number of an MCR in a cancer sample with the level of expression of biomarker(s) within an MCR or copy number of an MCR in a normal, control sample. A significant difference between the level of expression of biomarker(s) within an MCR or copy number of the MCR in the subject sample and the normal level is an indication that the subject is afflicted with cancer. The MCR is selected from the group consisting of those listed in Table 2.

Any biomarker or combination of biomarkers identified as described herein, or any MCR or combination of MCRs listed in Table 2, may be used in the compositions, kits, and methods of the present invention. In general, it is preferable to use biomarkers for which the difference between the amount, e.g., level of expression or copy number, and/or activity of the biomarker or MCR in cancer cells and the amount, e.g., level of expression or copy number, and/or activity of the same biomarker in normal cells, is as great as possible. Although this difference can be as small as the limit of detection of the method for assessing amount and/or activity of the biomarker, it is preferred that the difference be at least greater than the standard error of the assessment method, and preferably a difference of at least 2-, 3-, 4-, 5-, 6-, 7-, 8-, 9-, 10-, 15-, 20-, 25-, 100-, 500-, 1000-fold or greater than the amount, e.g., level of expression or copy number, and/or activity of the same biobiomarker in normal tissue.

It is understood that by routine screening of additional subject samples using one or more of the biomarkers of the invention, it will be realized that certain of the biomarkers have altered amount, structure, and/or activity in cancers of various types, including specific pancreatic cancers, e.g., pancreatic adenocarcinoma, as well as other cancers, examples of which include, but are not limited to, melanomas, breast cancer, bronchus cancer, colorectal cancer, prostate cancer, lung cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like.

For example, it will be confirmed that some of the biomarkers of the invention have altered amount, structure, and/or activity in some, i.e., 10%, 20%, 30%, or 40%, or most (i.e. 50% or more) or substantially all (i.e. 80% or more) of cancer, e.g., pancreatic adenocarcinoma. Furthermore, it will be confirmed that certain of the biomarkers of the invention are associated with cancer of various histologic subtypes.

In addition, as a greater number of subject samples are assessed for altered amount, structure, and/or activity of the biomarkers or altered expression or copy number MCRs of the invention and the outcomes of the individual subjects from whom the samples were obtained are correlated, it will also be confirmed that biomarkers have altered amount, structure, and/or activity of certain of the biomarkers or altered expression or copy number of MCRs of the invention are strongly correlated with malignant cancers and that altered expression of other biomarkers of the invention are strongly correlated with benign tumors or premalignant states. The compositions, kits, and methods of the invention are thus useful for characterizing one or more of the stage, grade, histological type, and benign/premalignant/malignant nature of cancer in subjects.

When the compositions, kits, and methods of the invention are used for characterizing one or more of the stage, grade, histological type, and benign/premalignant/malignant nature of cancer, in a subject, it is preferred that the biomarker or MCR or panel of biomarkers or MCRs of the invention be selected such that a positive result is obtained in at least about 20%, and preferably at least about 40%, 60%, or 80%, and more preferably, in substantially all, subjects afflicted with cancer, of the corresponding stage, grade, histological type, or benign/premalignant/malignant nature. Preferably, the biomarker or panel of biomarkers of the invention is selected such that a PPV (positive predictive value) of greater than about 10% is obtained for the general population (more preferably coupled with an assay specificity greater than 99.5%).

When a plurality of biomarkers or MCRs of the invention are used in the compositions, kits, and methods of the invention, the amount, structure, and/or activity of each biomarker or level of expression or copy number can be compared with the normal amount, structure, and/or activity of each of the plurality of biomarkers or level of expression or copy number, in non-cancerous samples of the same type, either in a single reaction mixture (i.e., using reagents, such as different fluorescent probes, for each biomarker) or in individual reaction mixtures corresponding to one or more of the biomarkers or MCRs.

In one embodiment, a significantly altered amount, structure, and/or activity of more than one of the plurality of biomarkers, or significantly altered copy number of one or more of the MCRs in the sample, relative to the corresponding normal levels, is an indication that the subject is afflicted with cancer. For example, a significantly lower copy number in the sample of each of the plurality of biomarkers or MCRs, relative to the corresponding normal levels or copy number, is an indication that the subject is afflicted with cancer. In yet another embodiment, a significantly enhanced copy number of one or more biomarkers or MCRs and a significantly lower level of expression or copy number of one or more biomarkers or MCRs in a sample relative to the corresponding normal levels, is an indication that the subject is afflicted with cancer. Also, for example, a significantly enhanced copy number in the sample of each of the plurality of biomarkers or MCRs, relative to the corresponding normal copy number, is an indication that the subject is afflicted with cancer. In yet another embodiment, a significantly enhanced copy number of one or more biomarkers or MCRs and a significantly lower copy number of one or more biomarkers or MCRs in a sample relative to the corresponding normal levels, is an indication that the subject is afflicted with cancer.

When a plurality of biomarkers or MCRs are used, it is preferred that 2, 3, 4, 5, 8, 10, 12, 15, 20, 30, or 50 or more individual biomarkers or MCRs be used or identified, wherein fewer biomarkers or MCRs are preferred.

Only a small number of biomarkers are known to be associated with, for example, pancreatic adenocarcinoma (e.g., p16^{INK4A} and TP53 tumor suppressors and the MYC, KRAS2 and AKT2 oncogenes). These biomarkers or other biomarkers which are known to be associated with other types of cancer may be used together with one or more biomarkers of the invention in, for example, a panel of biomarkers. In addition, frequent gains have been mapped to 3q, 5p, 7p, 8q, 11q, 12p, 17q and 20q and losses to 3p, 4q, 6q, 8p, 9p, 10q, 12q, 13q, 17p, 18q and 21q and 22q in pancreatic cancer. In some instances, validated oncogenes and tumor suppressor genes residing within these loci have been identified, including MYC (8q24), p16^{INK4A} (9p21), p53 (17p13), SMAD4 (18q21) and AKT2 (19q13). It is well known that certain types of genes, such as oncogenes, tumor suppressor genes, growth factor-like genes, protease-like genes, and protein kinase-like genes are often involved with development of cancers of various types. Thus, among the biomarkers of the invention, use of those which correspond to proteins which resemble known proteins encoded by known oncogenes and tumor suppressor genes, and those which correspond to proteins which resemble growth factors, proteases, and protein kinases, are preferred.

It is recognized that the compositions, kits, and methods of the invention will be of particular utility to subjects having an enhanced risk of developing cancer, and their medical advisors. Subjects recognized as having an enhanced risk of developing cancer, include, for example, subjects having a familial history of cancer, subjects identified as having a mutant oncogene (i.e. at least one allele), and subjects of advancing age.

An alteration, e.g. copy number, amount, structure, and/or activity of a biomarker in normal (i.e. non-cancerous) human tissue can be assessed in a variety of ways. In one embodiment, the normal level of expression or copy number is assessed by assessing the level of expression and/or copy number of the biomarker or MCR in a portion of cells which appear to be non-cancerous and by comparing this normal level of expression or copy number with the level of expression or copy number in a portion of the cells which are suspected of being cancerous. For example, when laparoscopy or other medical procedure, reveals the presence of a tumor on one portion of an organ, the normal level of expression or copy number of a biomarker or MCR may be assessed using the non-affected portion of the organ, and this normal level of expression or copy number may be compared with the level of expression or copy number of the same biomarker in an affected portion (i.e., the tumor) of the organ. Alternately, and particularly as further information becomes available as a result of routine performance of the methods described herein, population-average values for “normal” copy number, amount, structure, and/or activity of the biomarkers or MCRs of the invention may be used. In other embodiments, the “normal” copy number, amount, structure, and/or activity of a biomarker or MCR may be determined by assessing copy number, amount, structure, and/or activity of the biomarker or MCR in a subject sample obtained from a non-cancer-afflicted subject, from a subject sample obtained from a subject before the suspected onset of cancer in the subject, from archived subject samples, and the like.

The invention includes compositions, kits, and methods for assessing the presence of cancer cells in a sample (e.g. an archived tissue sample or a sample obtained from a subject). These compositions, kits, and methods are substantially the same as those described above, except that, where necessary, the compositions, kits, and methods are adapted for use with certain types of samples. For example, when the sample is a parafinized, archived human tissue sample, it may be necessary to adjust the ratio of compounds in the compositions of the invention, in the kits of the invention, or the methods used. Such methods are well known in the art and within the skill of the ordinary artisan.

The invention thus includes a kit for assessing the presence of cancer cells (e.g. in a sample such as a subject sample). The kit may comprise one or more reagents capable of identifying a biomarker or MCR of the invention, e.g., binding specifically with a nucleic acid or polypeptide corresponding to a biomarker or MCR of the invention. Suitable reagents for binding with a polypeptide corresponding to a biomarker of the invention include antibodies, antibody derivatives, antibody fragments, and the like. Suitable reagents for binding with a nucleic acid (e.g. a genomic DNA, an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

The kit of the invention may optionally comprise additional components useful for performing the methods of the invention. By way of example, the kit may comprise fluids (e.g., SSC buffer) suitable for annealing complementary nucleic acids or for binding an antibody with a protein with which it specifically binds, one or more sample compartments, an instructional material which describes performance of a method of the invention, a sample of normal cells, a sample of cancer cells, and the like.

A kit of the invention may comprise a reagent useful for determining protein level or protein activity of a biomarker. In another embodiment, a kit of the invention may comprise a reagent for determining methylation status of a biomarker, or may comprise a reagent for determining alteration of structure of a biomarker, e.g., the presence of a mutation.

The invention also includes a method of making an isolated hybridoma which produces an antibody useful in methods and kits of the present invention. A protein corresponding to a biomarker of the invention may be isolated (e.g. by purification from a cell in which it is expressed or by transcription and translation of a nucleic acid encoding the protein in vivo or in vitro using known methods) and a vertebrate, preferably a mammal such as a mouse, rat, rabbit, or sheep, is immunized using the isolated protein. The vertebrate may optionally (and preferably) be immunized at least one additional time with the isolated protein, so that the vertebrate exhibits a robust immune response to the protein. Splenocytes are isolated from the immunized vertebrate and fused with an immortalized cell line to form hybridomas, using any of a variety of methods well known in the art. Hybridomas formed in this manner are then screened using standard methods to identify one or more hybridomas which produce an antibody which specifically binds with the protein. The invention also includes hybridomas made by this method and antibodies made using such hybridomas.

The invention also includes a method of assessing the efficacy of a test compound for inhibiting cancer cells. As described above, differences in the amount, structure, and/or activity of the biomarkers of the invention, or level of expression or copy number of the MCRs of the invention, correlate with the cancerous state of cells. Although it is recognized that changes in the levels of amount, e.g., expression or copy number, structure, and/or activity of certain of the biomarkers or expression or copy number of the MCRs of the invention likely result from the cancerous state of cells, it is likewise recognized that changes in the amount may induce, maintain, and promote the cancerous state. Thus, compounds which inhibit cancer, in a subject may cause a change, e.g., a change in expression and/or activity of one or more of the biomarkers of the invention to a level nearer the normal level for that biomarker (e.g., the amount, e.g., expression, and/or activity for the biomarker in non-cancerous cells).

This method thus comprises comparing amount, e.g., expression, and/or activity of a biomarker in a first cell sample and maintained in the presence of the test compound and amount, e.g., expression, and/or activity of the biomarker in a second cell sample and maintained in the absence of the test compound. A significant increase in the amount, e.g., expression, and/or activity of a biomarker e.g., a biomarker that was shown to be decreased in cancer, a significant decrease in the amount, e.g., expression, and/or activity of a biomarker e.g., a biomarker that was shown to be increased in cancer, is an indication that the test compound inhibits cancer. The cell samples may, for example, be aliquots of a single sample of normal cells obtained from a subject, pooled samples of normal cells obtained from a subject, cells of a normal cell lines, aliquots of a single sample of cancer, cells obtained from a subject, pooled samples of cancer, cells obtained from a subject, cells of a cancer cell line, cells from an animal model of cancer, or the like. In one embodiment, the samples are cancer cells obtained from a subject and a plurality of compounds known to be effective for inhibiting various cancers, are tested in order to identify the compound which is likely to best inhibit the cancer in the subject.

This method may likewise be used to assess the efficacy of a therapy, e.g., chemotherapy, radiation therapy, surgery, or any other therapeutic approach useful for inhibiting cancer in a subject. In this method, the amount, e.g., expression, and/or activity of one or more biomarkers of the invention in a pair of samples (one subjected to the therapy, the other not subjected to the therapy) is assessed. As with the method of assessing the efficacy of test compounds, if the therapy induces a significant decrease in the amount, e.g., expression, and/or activity of a biomarker e.g., a biomarker that was shown to be increased in cancer, blocks induction of a biomarker e.g., a biomarker that was shown to be increased in cancer, or if the therapy induces a significant enhancement of the amount, e.g., expression, and/or activity of a biomarker e.g., a biomarker that was shown to be decreased in cancer, then the therapy is efficacious for inhibiting cancer. As above, if samples from a selected subject are used in this method, then alternative therapies can be assessed in vitro in order to select a therapy most likely to be efficacious for inhibiting cancer in the subject.

This method may likewise be used to monitor the progression of cancer in a subject, wherein if a sample in a subject has a significant decrease in the amount, e.g., expression, and/or activity of a biomarker e.g., a biomarker that was shown to be increased in cancer, or blocks induction of a biomarker e.g., a biomarker that was shown to be increased in cancer, or a significant enhancement of the amount, e.g., expression, and/or activity of a biomarker e.g., a biomarker that was shown to be decreased in cancer, during the progression of cancer, e.g., at a first point in time and a subsequent point in time, then the cancer has improved. In yet another embodiment, between the first point in time and a subsequent point in time, the subject has undergone treatment, e.g., chemotherapy, radiation therapy, surgery, or any other therapeutic approach useful for inhibiting cancer, has completed treatment, or is in remission.

As described herein, cancer in subjects is associated with an increase in amount, e.g., expression, and/or activity of one or more biomarker that was shown to be increased in cancer, and/or a decrease in amount, e.g., expression, and/or activity of one or more biomarker that was shown to be decreased in cancer. While, as discussed above, some of these changes in amount, e.g., expression, and/or activity number result from occurrence of the cancer, others of these changes induce, maintain, and promote the cancerous state of cancer cells. Thus, cancer characterized by an increase in the amount, e.g., expression, and/or activity of one or more biomarkers e.g., a biomarker that was shown to be increased in cancer, can be inhibited by inhibiting amount, e.g., expression, and/or activity of those biomarkers. Likewise, cancer characterized by a decrease in the amount, e.g., expression, and/or activity of one or more biomarkers e.g., a biomarker that was shown to be decreased in cancer, can be inhibited by enhancing amount, e.g., expression, and/or activity of those biomarkers.

Amount and/or activity of a biomarker e.g., a biomarker that was shown to be increased in cancer, can be inhibited in a number of ways generally known in the art. For example, an antisense oligonucleotide can be provided to the cancer cells in order to inhibit transcription, translation, or both, of the biomarker(s). An RNA interfering agent, e.g., an siRNA molecule, which is targeted to a biomarker e.g., a biomarker that was shown to be increased in cancer, can be provided to the cancer cells in order to inhibit expression of the target biomarker, e.g., through degradation or specific post-transcriptional gene silencing (PTGS) of the messenger RNA (mRNA) of the target biomarker. Alternately, a polynucleotide encoding an antibody, an antibody derivative, or an antibody fragment, e.g., a fragment capable of binding an antigen, and operably linked with an appropriate promoter or regulator region, can be provided to the cell in order to generate intracellular antibodies which will inhibit the function, amount, and/or activity of the protein corresponding to the biomarker(s). Conjugated antibodies or fragments thereof, e.g., chemolabeled antibodies, radiolabeled antibodies, or immunotoxins targeting a biomarker of the invention may also be administered to treat, prevent or inhibit cancer.

A small molecule may also be used to modulate, e.g., inhibit, expression and/or activity of a biomarker e.g., a biomarker that was shown to be increased in cancer. In one embodiment, a small molecule functions to disrupt a protein-protein interaction between a biomarker of the invention and a target molecule or ligand, thereby modulating, e.g., increasing or decreasing the activity of the biomarker.

Using the methods described herein, a variety of molecules, particularly including molecules sufficiently small that they are able to cross the cell membrane, can be screened in order to identify molecules which inhibit amount and/or activity of the biomarker(s). The compound so identified can be provided to the subject in order to inhibit amount and/or activity of the biomarker(s) in the cancer cells of the subject.

Amount and/or activity of a biomarker e.g., a biomarker that was shown to be decreased in cancer, can be enhanced in a number of ways generally known in the art. For example, a polynucleotide encoding the biomarker and operably linked with an appropriate promoter/regulator region can be provided to cells of the subject in order to induce enhanced expression and/or activity of the protein (and mRNA) corresponding to the biomarker therein. Alternatively, if the protein is capable of crossing the cell membrane, inserting itself in the cell membrane, or is normally a secreted protein, then amount and/or activity of the protein can be enhanced by providing the protein (e.g., directly or by way of the bloodstream) to cancer cells in the subject. A small molecule may also be used to modulate, e.g., increase, expression or activity of a biomarker e.g., a biomarker that was shown to be decreased in cancer. Furthermore, in another embodiment, a modulator of a biomarker of the invention, e.g., a small molecule, may be used, for example, to re-express a silenced gene, e.g., a tumor suppressor, in order to treat or prevent cancer. For example, such a modulator may interfere with a DNA binding element or a methyltransferase.

As described above, the cancerous state of human cells is correlated with changes in the amount and/or activity of the biomarkers of the invention. Thus, compounds which induce increased expression or activity of one or more of the biomarker that was shown to be increased in cancer, decreased amount and/or activity of one or more of the biomarkers that was shown to be decreased in cancer, can induce cell carcinogenesis. The invention also includes a method for assessing the human cell carcinogenic potential of a test compound. This method comprises maintaining separate aliquots of human cells in the presence and absence of the test compound. Expression or activity of a biomarker of the invention in each of the aliquots is compared. A significant increase in the amount and/or activity of a biomarker e.g., a biomarker that was shown to be increased in cancer, or a significant decrease in the amount and/or activity of a biomarker e.g., a biomarker that was shown to be decreased in cancer, in the aliquot maintained in the presence of the test compound (relative to the aliquot maintained in the absence of the test compound) is an indication that the test compound possesses human cell carcinogenic potential. The relative carcinogenic potentials of various test compounds can be assessed by comparing the degree of enhancement or inhibition of the amount and/or activity of the relevant biomarkers, by comparing the number of biomarkers for which the amount and/or activity is enhanced or inhibited, or by comparing both.

### IV. ISOLATED NUCLEIC ACID MOLECULES

One aspect of the invention pertains to isolated nucleic acid molecules that correspond to a biomarker of the invention, including nucleic acids which encode a polypeptide corresponding to a biomarker of the invention or a portion of such a polypeptide. The nucleic acid molecules of the invention include those nucleic acid molecules which reside in the MCRs identified herein. Isolated nucleic acid molecules of the invention also include nucleic acid molecules sufficient for use as hybridization probes to identify nucleic acid molecules that correspond to a biomarker of the invention, including nucleic acid molecules which encode a polypeptide corresponding to a biomarker of the invention, and fragments of such nucleic acid molecules, e.g., those suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

A nucleic acid molecule of the present invention, e.g., a nucleic acid molecules encoding a protein corresponding to a biomarker listed in Tables 1 or 2, can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which has a nucleotide sequence complementary to the nucleotide sequence of a nucleic acid corresponding to a biomarker of the invention or to the nucleotide sequence of a nucleic acid encoding a protein which corresponds to a biomarker of the invention. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence, wherein the full length nucleic acid sequence comprises a biomarker of the invention or which encodes a polypeptide corresponding to a biomarker of the invention. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences corresponding to one or more biomarkers of the invention. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

The invention further encompasses nucleic acid molecules that differ, due to degeneracy of the genetic code, from the nucleotide sequence of nucleic acid molecules encoding a protein which corresponds to a biomarker of the invention, and thus encode the same protein.

It will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

The term “allele,” which is used interchangeably herein with “allelic variant,” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. Alleles of a specific gene can differ from each other in a single nucleotide, or several nucleotides, and can include substitutions, deletions, and insertions of nucleotides. An allele of a gene can also be a form of a gene containing one or more mutations.

The term “allelic variant of a polymorphic region of gene” or “allelic variant”, used interchangeably herein, refers to an alternative form of a gene having one of several possible nucleotide sequences found in that region of the gene in the population. As used herein, allelic variant is meant to encompass functional allelic variants, non-functional allelic variants, SNPs, mutations and polymorphisms.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. SNP's may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, or it may have no effect on the function of the protein.

As used herein, the terms “gene” and “recombinant gene” refer to nucleic acid molecules comprising an open reading frame encoding a polypeptide corresponding to a biomarker of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, an isolated nucleic acid molecule of the invention is at least 7, 15, 20, 25, 30, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to a nucleic acid molecule corresponding to a biomarker of the invention or to a nucleic acid molecule encoding a protein corresponding to a biomarker of the invention. As used herein, the term “hybridizes under stringent conditions” is intended to describe conditions for hybridization and washing under which nucleotide sequences at least 60% (65%, 70%, 75%, 80%, preferably 85%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

In addition to naturally-occurring allelic variants of a nucleic acid molecule of the invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a polypeptide of the invention that contain changes in amino acid residues that are not essential for activity. Such polypeptides differ in amino acid sequence from the naturally-occurring proteins which correspond to the biomarkers of the invention, yet retain biological activity. In one embodiment, such a protein has an amino acid sequence that is at least about 40% identical, 50%, 60%, 70%, 80%, 90%, 95%, or 98% identical to the amino acid sequence of one of the proteins which correspond to the biomarkers of the invention.

An isolated nucleic acid molecule encoding a variant protein can be created by introducing one or more nucleotide substitutions, additions or deletions into the nucleotide sequence of nucleic acids of the invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

The present invention encompasses antisense nucleic acid molecules, i.e., molecules which are complementary to a sense nucleic acid of the invention, e.g., complementary to the coding strand of a double-stranded cDNA molecule corresponding to a biomarker of the invention or complementary to an mRNA sequence corresponding to a biomarker of the invention. Accordingly, an antisense nucleic acid molecule of the invention can hydrogen bond to (i.e. anneal with) a sense nucleic acid of the invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the invention. The non-coding regions (“5′ and 3′untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a polypeptide corresponding to a selected biomarker of the invention to thereby inhibit expression of the biomarker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the invention includes direct injection at a tissue site or infusion of the antisense nucleic acid into a pancreatic-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual α-units, the strands run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a polypeptide corresponding to a biomarker of the invention can be designed based upon the nucleotide sequence of a cDNA corresponding to the biomarker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the invention can be used to select a catalytic RNA having a specific ribonuclease activity from a pool of RNA molecules (see, e.g., Bartel and Szostak, 1993, Science 261:1411-1418).

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acid molecules (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., or targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

The invention also includes molecular beacon nucleic acid molecules having at least one region which is complementary to a nucleic acid molecule of the invention, such that the molecular beacon is useful for quantitating the presence of the nucleic acid molecule of the invention in a sample. A “molecular beacon” nucleic acid is a nucleic acid molecule comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid molecules are not annealed with one another, fluorescence of the fluorophore is quenched to a lesser degree. Molecular beacon nucleic acid molecules are described, for example, in U.S. Pat. No. 5,876,930.

### V. ISOLATED PROTEINS AND ANTIBODIES

One aspect of the invention pertains to isolated proteins which correspond to individual biomarkers of the invention, and biologically active portions thereof, as well as polypeptide fragments suitable for use as immunogens to raise antibodies directed against a polypeptide corresponding to a biomarker of the invention. In one embodiment, the native polypeptide corresponding to a biomarker can be isolated from cells or tissue sources by an appropriate purification scheme using standard protein purification techniques. In another embodiment, polypeptides corresponding to a biomarker of the invention are produced by recombinant DNA techniques. Alternative to recombinant expression, a polypeptide corresponding to a biomarker of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a polypeptide corresponding to a biomarker of the invention include polypeptides comprising amino acid sequences sufficiently identical to or derived from the amino acid sequence of the protein corresponding to the biomarker which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides have an amino acid sequence of a protein encoded by a nucleic acid molecule of a biomarker of the invention. Other useful proteins are substantially identical (e.g., at least about 40%, preferably 50%, 60%, 70%, 80%, 90%, 95%, or 99%) to one of these sequences and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) Comput Appl Biosci, 4:11-7. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The invention also provides chimeric or fusion proteins corresponding to a biomarker of the invention. As used herein, a “chimeric protein” or “fusion protein” comprises all or part (preferably a biologically active part) of a polypeptide corresponding to a biomarker of the invention operably linked to a heterologous polypeptide (i.e., a polypeptide other than the polypeptide corresponding to the biomarker). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention.

One useful fusion protein is a GST fusion protein in which a polypeptide corresponding to a biomarker of the invention is fused to the carboxyl terminus of GST sequences. Such fusion proteins can facilitate the purification of a recombinant polypeptide of the invention.

In another embodiment, the fusion protein contains a heterologous signal sequence at its amino terminus. For example, the native signal sequence of a polypeptide corresponding to a biomarker of the invention can be removed and replaced with a signal sequence from another protein. For example, the gp67 secretory sequence of the baculovirus envelope protein can be used as a heterologous signal sequence (Ausubel et al., ed., Current Protocols in Molecular Biology, John Wiley & Sons, NY, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide corresponding to a biomarker of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction can be useful therapeutically, both for treating proliferative and differentiative disorders and for modulating (e.g. promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the polypeptides corresponding to individual biomarkers of the invention. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a polypeptide corresponding to a biomarker of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan, 1992, Proc. Natl. Acad. Sci. USA 89:7811-7815; Delgrave et al., 1993, Protein Engineering 6(3):327-331).

An isolated polypeptide corresponding to a biomarker of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of one of the polypeptides of the invention, and encompasses an epitope of the protein such that an antibody raised against the peptide forms a specific immune complex with a biomarker of the invention to which the protein corresponds. Preferred epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions.

An immunogen typically is used to prepare antibodies by immunizing a suitable (i.e. immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. The antibody titer in the immunized subject can be monitored over time by standard techniques, such as with an enzyme linked immunosorbent assay (ELISA) using immobilized polypeptide. If desired, the antibody molecules can be harvested or isolated from the subject (e.g., from the blood or serum of the subject) and further purified by well-known techniques, such as protein A chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human subjects. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to a biomarker of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

An antibody, antibody derivative, or fragment thereof, which specifically binds a biomarker of the invention which is overexpressed in cancer may be used to inhibit activity of a biomarker, and therefore may be administered to a subject to treat, inhibit, or prevent cancer in the subject. Furthermore, conjugated antibodies may also be used to treat, inhibit, or prevent cancer in a subject. Conjugated antibodies, preferably monoclonal antibodies, or fragments thereof, are antibodies which are joined to drugs, toxins, or radioactive atoms, and used as delivery vehicles to deliver those substances directly to cancer cells. The antibody, e.g., an antibody which specifically binds a biomarker of the invention (e.g., a biomarker listed in Table 2), is administered to a subject and binds the biomarker, thereby delivering the toxic substance to the cancer cell, minimizing damage to normal cells in other parts of the body.

Conjugated antibodies are also referred to as “tagged,” “labeled,” or “loaded.” Antibodies with chemotherapeutic agents attached are generally referred to as chemolabeled. Examples of chemotherapeutic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Antibodies with radioactive particles attached are referred to as radiolabeled, and this type of therapy is known as radioimmunotherapy (RIT). Aside from being used to treat cancer, radiolabeled antibodies can also be used to detect areas of cancer spread in the body. Antibodies attached to toxins are called immunotoxins.

Immunotoxins are made by attaching toxins (e.g., poisonous substances from plants or bacteria) to monoclonal antibodies. Immunotoxins may be produced by attaching monoclonal antibodies to bacterial toxins such as ribosome-inhibiting protein (see Better et al., U.S. Pat. No. 6,146,631, the disclosure of which is incorporated herein in its entirety), abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors diphtherial toxin (DT) or pseudomonal exotoxin (PE40), or to plant toxins such as ricin A or saporin.

An antibody directed against a polypeptide corresponding to a biomarker of the invention (e.g., a monoclonal antibody) can be used to isolate the polypeptide by standard techniques, such as affinity chromatography or immunoprecipitation. Moreover, such an antibody can be used to detect the biomarker (e.g., in a cellular lysate or cell supernatant) in order to evaluate the level and pattern of expression of the biomarker. The antibodies can also be used diagnostically to monitor protein levels in tissues or body fluids (e.g. in a pancreas-associated body fluid, such as bile) as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### VI. RECOMBINANT EXPRESSION VECTORS AND HOST CELLS

Another aspect of the invention pertains to vectors, preferably expression vectors, containing a nucleic acid encoding a polypeptide corresponding to a biomarker of the invention (or a portion of such a polypeptide). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide corresponding to a biomarker of the invention in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET 11d (Studier et al., p. 60-89, In Gene Expression Technology: Methods in Enzymology vol. 185, Academic Press, San Diego, Calif., 1991). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21 (DE3) or HMS174(DE3) from a resident prophage harboring a T7 gn1 gene under the transcriptional control of the lacUV 5 promoter.

One strategy to maximize recombinant protein expression in E. coli is to express the protein in a host bacterium with an impaired capacity to proteolytically cleave the recombinant protein (Gottesman, p. 119-128, In Gene Expression Technology. Methods in Enzymology vol. 185, Academic Press, San Diego, Calif., 1990. Another strategy is to alter the nucleic acid sequence of the nucleic acid to be inserted into an expression vector so that the individual codons for each amino acid are those preferentially utilized in E. coli (Wada et al., 1992, Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari et al., 1987, EMBO J. 6:229-234), pMFa (Kurjan and Herskowitz, 1982, Cell 30:933-943), pJRY88 (Schultz et al., 1987, Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al., 1983, Mol. Cell. Biol. 3:2156-2165) and the pVL series (Lucklow and Summers, 1989, Virology 170:31-39).

In yet another embodiment, a nucleic acid of the invention is expressed in mammalian cells using a mammalian expression vector. Examples of mammalian expression vectors include pCDM8 (Seed, 1987, Nature 329:840) and pMT2PC (Kaufman et al., 1987, EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Banerji et al., 1983, Cell 33:729-740; Queen and Baltimore, 1983, Cell 33:741-748), neuron-specific promoters (e.g., the neurofilament promoter; Byrne and Ruddle, 1989, Proc. Natl. Acad. Sci. USA 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Camper and Tilghman, 1989, Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., 1986, Trends in Genetics, Vol. 1(1).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable biomarker (e.g., for resistance to antibiotics) is generally introduced into the host cells along with the gene of interest. Preferred selectable biomarkers include those which confer resistance to drugs, such as G418, hygromycin and methotrexate. Cells stably transfected with the introduced nucleic acid can be identified by drug selection (e.g., cells that have incorporated the selectable biomarker gene will survive, while the other cells die).

A host cell of the invention, such as a prokaryotic or eukaryotic host cell in culture, can be used to produce a polypeptide corresponding to a biomarker of the invention. Accordingly, the invention further provides methods for producing a polypeptide corresponding to a biomarker of the invention using the host cells of the invention. In one embodiment, the method comprises culturing the host cell of invention (into which a recombinant expression vector encoding a polypeptide of the invention has been introduced) in a suitable medium such that the biomarker is produced. In another embodiment, the method further comprises isolating the biomarker polypeptide from the medium or the host cell.

The host cells of the invention can also be used to produce nonhuman transgenic animals. For example, in one embodiment, a host cell of the invention is a fertilized oocyte or an embryonic stem cell into which sequences encoding a polypeptide corresponding to a biomarker of the invention have been introduced. Such host cells can then be used to create non-human transgenic animals in which exogenous sequences encoding a biomarker protein of the invention have been introduced into their genome or homologous recombinant animals in which endogenous gene(s) encoding a polypeptide corresponding to a biomarker of the invention sequences have been altered. Such animals are useful for studying the function and/or activity of the polypeptide corresponding to the biomarker, for identifying and/or evaluating modulators of polypeptide activity, as well as in pre-clinical testing of therapeutics or diagnostic molecules, for biomarker discovery or evaluation, e.g., therapeutic and diagnostic biomarker discovery or evaluation, or as surrogates of drug efficacy and specificity.

As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal. Transgenic animals also include inducible transgenic animals, such as those described in, for example, Chan I. T., et al. (2004) J Clin Invest. 113(4):528-38 and Chin L. et al (1999) Nature 400(6743):468-72.

A transgenic animal of the invention can be created by introducing a nucleic acid encoding a polypeptide corresponding to a biomarker of the invention into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, Cold Spring Harbor Laboratory Press, Cold Spring Harbor, N.Y., 1986. Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create an homologous recombinant animal, a vector is prepared which contains at least a portion of a gene encoding a polypeptide corresponding to a biomarker of the invention into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g., Thomas and Capecchi, 1987, Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al., 1992, Cell 69:915). The selected cells are then injected into a blastocyst of an animal (e.g., a mouse) to form aggregation chimeras (see, e.g., Bradley, Teratocarcinomas and Embryonic Stem Cells: A Practical Approach, Robertson, Ed., IRL, Oxford, 1987, pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication NOS. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al., 1991, Science 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication NOS. WO 97/07668 and WO 97/07669.

### VII. METHODS OF TREATMENT

The present invention provides for both prophylactic and therapeutic methods of treating a subject, e.g., a human, who has or is at risk of (or susceptible to) cancer, e.g., pancreatic cancer, e.g., pancreatic adenocarcinoma. As used herein, “treatment” of a subject includes the application or administration of a therapeutic agent to a subject, or application or administration of a therapeutic agent to a cell or tissue from a subject, who has a diseases or disorder, has a symptom of a disease or disorder, or is at risk of (or susceptible to) a disease or disorder, with the purpose of curing, inhibiting, healing, alleviating, relieving, altering, remedying, ameliorating, improving, or affecting the disease or disorder, the symptom of the disease or disorder, or the risk of (or susceptibility to) the disease or disorder. As used herein, a “therapeutic agent” or “compound” includes, but is not limited to, small molecules, peptides, peptidomimetics, polypeptides, RNA interfering agents, e.g., siRNA molecules, antibodies, ribozymes, and antisense oligonucleotides.

As described herein, cancer in subjects is associated with a change, e.g., an increase in the amount and/or activity, or a change in the structure, of one or more biomarkers, e.g., a biomarker that was shown to be increased in cancer, and/or a decrease in the amount and/or activity, or a change in the structure of one or more biomarkers, e.g., a biomarker that was shown to be decreased in cancer. While, as discussed above, some of these changes in amount, structure, and/or activity, result from occurrence of the cancer, others of these changes induce, maintain, and promote the cancerous state of cancer, cells. Thus, cancer, characterized by an increase in the amount and/or activity, or a change in the structure, of one or more biomarkers, e.g., a biomarker that is shown to be increased in cancer, can be inhibited by inhibiting amount, e.g., expression or protein level, and/or activity of those biomarkers. Likewise, cancer characterized by a decrease in the amount and/or activity, or a change in the structure, of one or more biomarkers, e.g., a biomarker that is shown to be decreased in cancer, can be inhibited by enhancing amount, e.g., expression or protein level, and/or activity of those biomarkers

Accordingly, another aspect of the invention pertains to methods for treating a subject suffering from cancer. These methods involve administering to a subject a compound which modulates amount and/or activity of one or more biomarkers of the invention. For example, methods of treatment or prevention of cancer include administering to a subject a compound which decreases the amount and/or activity of one or more biomarkers, e.g., a biomarker that was shown to be increased in cancer. Compounds, e.g., antagonists, which may be used to inhibit amount and/or activity of a biomarker, e.g., a biomarker that was shown to be increased in cancer, to thereby treat or prevent cancer include antibodies (e.g., conjugated antibodies), small molecules, RNA interfering agents, e.g., siRNA molecules, ribozymes, and antisense oligonucleotides. In one embodiment, an antibody used for treatment is conjugated to a toxin, a chemotherapeutic agent, or radioactive particles.

Methods of treatment or prevention of cancer also include administering to a subject a compound which increases the amount and/or activity of one or more biomarkers, e.g., a biomarker that was shown to be decreased in cancer. Compounds, e.g., agonists, which may be used to increase expression or activity of a biomarker, e.g., a biomarker that was shown to be decreased in cancer, to thereby treat or prevent cancer include small molecules, peptides, peptoids, peptidomimetics, and polypeptides.

Small molecules used in the methods of the invention include those which inhibit a protein-protein interaction and thereby either increase or decrease biomarker amount and/or activity. Furthermore, modulators, e.g., small molecules, which cause re-expression of silenced genes, e.g., tumor suppressors, are also included herein. For example, such molecules include compounds which interfere with DNA binding or methyltransferase activity.

An aptamer may also be used to modulate, e.g., increase or inhibit expression or activity of a biomarker of the invention to thereby treat, prevent or inhibit cancer. Aptamers are DNA or RNA molecules that have been selected from random pools based on their ability to bind other molecules. Aptamers may be selected which bind nucleic acids or proteins.

### VIII. PHARMACEUTICAL COMPOSITIONS

The small molecules, peptides, peptoids, peptidomimetics, polypeptides, RNA interfering agents, e.g., siRNA molecules, antibodies, ribozymes, and antisense oligonucleotides (also referred to herein as “active compounds” or “compounds”) corresponding to a biomarker of the invention can be incorporated into pharmaceutical compositions suitable for administration. Such compositions typically comprise the small molecules, peptides, peptoids, peptidomimetics, polypeptides, RNA interfering agents, e.g., siRNA molecules, antibodies, ribozymes, or antisense oligonucleotides and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid corresponding to a biomarker of the invention. Such methods comprise formulating a pharmaceutically acceptable carrier with an agent which modulates expression or activity of a polypeptide or nucleic acid corresponding to a biomarker of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid corresponding to a biomarker of the invention and one or more additional active compounds.

It is understood that appropriate doses of small molecule agents and protein or polypeptide agents depends upon a number of factors within the knowledge of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid molecule or polypeptide of the invention. Small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses of a small molecule include milligram or microgram amounts per kilogram of subject or sample weight (e.g. about 1 microgram per kilogram to about 500 milligrams per kilogram, about 100 micrograms per kilogram to about 5 milligrams per kilogram, or about 1 microgram per kilogram to about 50 micrograms per kilogram).

As defined herein, a therapeutically effective amount of an RNA interfering agent, e.g., siRNA, (i.e., an effective dosage) ranges from about 0.001 to 3,000 mg/kg body weight, preferably about 0.01 to 2500 mg/kg body weight, more preferably about 0.1 to 2000, about 0.1 to 1000 mg/kg body weight, 0.1 to 500 mg/kg body weight, 0.1 to 100 mg/kg body weight, 0.1 to 50 mg/kg body weight, 0.1 to 25 mg/kg body weight, and even more preferably about 1 to 10 mg/kg, 2 to 9 mg/kg, 3 to 8 mg/kg, 4 to 7 mg/kg, or 5 to 6 mg/kg body weight. Treatment of a subject with a therapeutically effective amount of an RNA interfering agent can include a single treatment or, preferably, can include a series of treatments. In a preferred example, a subject is treated with an RNA interfering agent in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks.

Exemplary doses of a protein or polypeptide include gram, milligram or microgram amounts per kilogram of subject or sample weight (e.g. about 1 microgram per kilogram to about 5 grams per kilogram, about 100 micrograms per kilogram to about 500 milligrams per kilogram, or about 1 milligram per kilogram to about 50 milligrams per kilogram). It is furthermore understood that appropriate doses of one of these agents depend upon the potency of the agent with respect to the expression or activity to be modulated. Such appropriate doses can be determined using the assays described herein. When one or more of these agents is to be administered to an animal (e.g. a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes having monoclonal antibodies incorporated therein or thereon) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the epithelium). A method for lipidation of antibodies is described by Cruikshank et al. (1997) J. Acquired Immune Deficiency Syndromes and Human Retrovirology 14:193.

The nucleic acid molecules corresponding to a biomarker of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al., 1994, Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The RNA interfering agents, e.g., siRNAs used in the methods of the invention can be inserted into vectors. These constructs can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the vector can include the RNA interfering agent, e.g., the siRNA vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### IX. PREDICTIVE MEDICINE

The present invention also pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining the amount, structure, and/or activity of polypeptides or nucleic acids corresponding to one or more biomarkers of the invention, in order to determine whether an individual is at risk of developing cancer. Such assays can be used for prognostic or predictive purposes to thereby prophylactically treat an individual prior to the onset of the cancer.

Yet another aspect of the invention pertains to monitoring the influence of agents (e.g., drugs or other compounds administered either to inhibit cancer or to treat or prevent any other disorder {i.e. in order to understand any carcinogenic effects that such treatment may have}) on the amount, structure, and/or activity of a biomarker of the invention in clinical trials. These and other agents are described in further detail in the following sections.

A. Diagnostic Assays

1. Methods for Detection of Copy Number

Methods of evaluating the copy number of a particular biomarker or chromosomal region (e.g., an MCR) are well known to those of skill in the art. The presence or absence of chromosomal gain or loss can be evaluated simply by a determination of copy number of the regions or biomarkers identified herein.

Methods for evaluating copy number of encoding nucleic acid in a sample include, but are not limited to, hybridization-based assays. For example, one method for evaluating the copy number of encoding nucleic acid in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal mRNA (e.g., a non-amplified portion of the same or related cell, tissue, organ, etc.) provides an estimate of the relative copy number of the target nucleic acid.

An alternative means for determining the copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

Preferred hybridization-based assays include, but are not limited to, traditional “direct probe” methods such as Southern blots or in situ hybridization (e.g., FISH and FISH plus SKY), and “comparative probe” methods such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained.

The probes are typically labeled, e.g., with radioisotopes or fluorescent reporters. Preferred probes are sufficiently long so as to specifically hybridize with the target nucleic acid(s) under stringent conditions. The preferred size range is from about 200 bases to about 1000 bases.

In some applications it is necessary to block the hybridization capacity of repetitive sequences. Thus, in some embodiments, tRNA, human genomic DNA, or Cot-I DNA is used to block non-specific hybridization.

In CGH methods, a first collection of nucleic acids (e.g., from a sample, e.g., a possible tumor) is labeled with a first label, while a second collection of nucleic acids (e.g., a control, e.g., from a healthy cell/tissue) is labeled with a second label. The ratio of hybridization of the nucleic acids is determined by the ratio of the two (first and second) labels binding to each fiber in the array. Where there are chromosomal deletions or multiplications, differences in the ratio of the signals from the two labels will be detected and the ratio will provide a measure of the copy number. Array-based CGH may also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays.

Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), etc. In one embodiment, the hybridization protocol of Pinkel, et al. (1998) Nature Genetics 20: 207-211, or of Kallioniemi (1992) Proc. Natl. Acad Sci USA 89:5321-5325 (1992) is used.

The methods of the invention are particularly well suited to array-based hybridization formats. Array-based CGH is described in U.S. Pat. No. 6,455,258, the contents of which are incorporated herein by reference.

In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the invention. In fluorogenic quantitative PCR, quantitation is based on amount of fluorescence signals, e.g., TaqMan and sybr green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

Loss of heterozygosity (LOH) mapping (Wang, Z. C., et al. (2004) Cancer Res 64(1):64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93) may also be used to identify regions of amplification or deletion.

2. Methods for Detection of Gene Expression

Biomarker expression level can also be assayed as a method for diagnosis of cancer or risk for developing cancer. Expression of a biomarker of the invention may be assessed by any of a wide variety of well known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g. mRNA), by a measure of the quantity of translated protein, or by a measure of gene product activity. Biomarker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

Methods of detecting and/or quantifying the gene transcript (mRNA or cDNA made therefrom) using nucleic acid hybridization techniques are known to those of skill in the art (see Sambrook et al. supra). For example, one method for evaluating the presence, absence, or quantity of cDNA involves a Southern transfer as described above. Briefly, the mRNA is isolated (e.g. using an acid guanidinium-phenol-chloroform extraction method, Sambrook et al. supra.) and reverse transcribed to produce cDNA. The cDNA is then optionally digested and run on a gel in buffer and transferred to membranes. Hybridization is then carried out using the nucleic acid probes specific for the target cDNA.

A general principle of such diagnostic and prognostic assays involves preparing a sample or reaction mixture that may contain a biomarker, and a probe, under appropriate conditions and for a time sufficient to allow the biomarker and probe to interact and bind, thus forming a complex that can be removed and/or detected in the reaction mixture. These assays can be conducted in a variety of ways.

For example, one method to conduct such an assay would involve anchoring the biomarker or probe onto a solid phase support, also referred to as a substrate, and detecting target biomarker/probe complexes anchored on the solid phase at the end of the reaction. In one embodiment of such a method, a sample from a subject, which is to be assayed for presence and/or concentration of biomarker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, biomarker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the biomarker or probe belongs. Well-known supports or carriers include, but are not limited to, glass, polystyrene, nylon, polypropylene, polyethylene, dextran, amylases, natural and modified celluloses, polyacrylamides, gabbros, and magnetite.

In order to conduct assays with the above-mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of biomarker/probe complexes anchored to the solid phase can be accomplished in a number of methods outlined herein.

In a preferred embodiment, the probe, when it is the unanchored assay component, can be labeled for the purpose of detection and readout of the assay, either directly or indirectly, with detectable labels discussed herein and which are well-known to one skilled in the art.

It is also possible to directly detect biomarker/probe complex formation without further manipulation or labeling of either component (biomarker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a biomarker can be accomplished without labeling either assay component (probe or biomarker) by utilizing a technology such as real-time Biomolecular Interaction Analysis (BIA) (see, e.g., Sjolander, S, and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with biomarker and probe as solutes in a liquid phase. In such an assay, the complexed biomarker and probe are separated from uncomplexed components by any of a number of standard techniques, including but not limited to: differential centrifugation, chromatography, electrophoresis and immunoprecipitation. In differential centrifugation, biomarker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the biomarker/probe complex as compared to the uncomplexed components may be exploited to differentiate the complex from uncomplexed components, for example, through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of mRNA corresponding to the biomarker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated nucleic acid can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a biomarker of the present invention. Other suitable probes for use in the diagnostic assays of the invention are described herein. Hybridization of an mRNA with the probe indicates that the biomarker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the biomarkers of the present invention.

The probes can be full length or less than the full length of the nucleic acid sequence encoding the protein. Shorter probes are empirically tested for specificity. Preferably nucleic acid probes are 20 bases or longer in length. (See, e.g., Sambrook et al. for methods of selecting nucleic acid probe sequences for use in nucleic acid hybridization.) Visualization of the hybridized portions allows the qualitative determination of the presence or absence of cDNA.

An alternative method for determining the level of a transcript corresponding to a biomarker of the present invention in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. Fluorogenic rtPCR may also be used in the methods of the invention. In fluorogenic rtPCR, quantitation is based on amount of fluorescence signals, e.g., TaqMan and Sybr green. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to mRNA that encodes the biomarker.

As an alternative to making determinations based on the absolute expression level of the biomarker, determinations may be based on the normalized expression level of the biomarker. Expression levels are normalized by correcting the absolute expression level of a biomarker by comparing its expression to the expression of a gene that is not a biomarker, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a non-cancerous sample, or between samples from different sources.

Alternatively, the expression level can be provided as a relative expression level. To determine a relative expression level of a biomarker, the level of expression of the biomarker is determined for 10 or more samples of normal versus cancer cell isolates, preferably 50 or more samples, prior to the determination of the expression level for the sample in question. The mean expression level of each of the genes assayed in the larger number of samples is determined and this is used as a baseline expression level for the biomarker. The expression level of the biomarker determined for the test sample (absolute level of expression) is then divided by the mean expression value obtained for that biomarker. This provides a relative expression level.

Preferably, the samples used in the baseline determination will be from cancer cells or normal cells of the same tissue type. The choice of the cell source is dependent on the use of the relative expression level. Using expression found in normal tissues as a mean expression score aids in validating whether the biomarker assayed is specific to the tissue from which the cell was derived (versus normal cells). In addition, as more data is accumulated, the mean expression value can be revised, providing improved relative expression values based on accumulated data. Expression data from normal cells provides a means for grading the severity of the cancer state.

In another preferred embodiment, expression of a biomarker is assessed by preparing genomic DNA or mRNA/cDNA (i.e. a transcribed polynucleotide) from cells in a subject sample, and by hybridizing the genomic DNA or mRNA/cDNA with a reference polynucleotide which is a complement of a polynucleotide comprising the biomarker, and fragments thereof. cDNA can, optionally, be amplified using any of a variety of polymerase chain reaction methods prior to hybridization with the reference polynucleotide. Expression of one or more biomarkers can likewise be detected using quantitative PCR (QPCR) to assess the level of expression of the biomarker(s). Alternatively, any of the many known methods of detecting mutations or variants (e.g., single nucleotide polymorphisms, deletions, etc.) of a biomarker of the invention may be used to detect occurrence of a mutated biomarker in a subject.

In a related embodiment, a mixture of transcribed polynucleotides obtained from the sample is contacted with a substrate having fixed thereto a polynucleotide complementary to or homologous with at least a portion (e.g. at least 7, 10, 15, 20, 25, 30, 40, 50, 100, 500, or more nucleotide residues) of a biomarker of the invention. If polynucleotides complementary to or homologous with are differentially detectable on the substrate (e.g., detectable using different chromophores or fluorophores, or fixed to different selected positions), then the levels of expression of a plurality of biomarkers can be assessed simultaneously using a single substrate (e.g., a “gene chip” microarray of polynucleotides fixed at selected positions). When a method of assessing biomarker expression is used which involves hybridization of one nucleic acid with another, it is preferred that the hybridization be performed under stringent hybridization conditions.

In another embodiment, a combination of methods to assess the expression of a biomarker is utilized.

Because the compositions, kits, and methods of the invention rely on detection of a difference in expression levels or copy number of one or more biomarkers of the invention, it is preferable that the level of expression or copy number of the biomarker is significantly greater than the minimum detection limit of the method used to assess expression or copy number in at least one of normal cells and cancerous cells.

3. Methods for Detection of Expressed Protein

The activity or level of a biomarker protein can also be detected and/or quantified by detecting or quantifying the expressed polypeptide. The polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, and the like. A skilled artisan can readily adapt known protein/antibody detection methods for use in determining whether cells express a biomarker of the present invention.

A preferred agent for detecting a polypeptide of the invention is an antibody capable of binding to a polypeptide corresponding to a biomarker of the invention, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In a preferred embodiment, the antibody is labeled, e.g., a radio-labeled, chromophore-labeled, fluorophore-labeled, or enzyme-labeled antibody. In another embodiment, an antibody derivative (e.g. an antibody conjugated with a substrate or with the protein or ligand of a protein-ligand pair {e.g. biotin-streptavidin}), or an antibody fragment (e.g. a single-chain antibody, an isolated antibody hypervariable domain, etc.) which binds specifically with a protein corresponding to the biomarker, such as the protein encoded by the open reading frame corresponding to the biomarker or such a protein which has undergone all or a portion of its normal post-translational modification, is used.

Proteins from cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

In one format, antibodies, or antibody fragments, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means. Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of a polypeptide in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind a polypeptide. The anti-polypeptide antibodies specifically bind to the polypeptide on the solid support. These antibodies may be directly labeled or alternatively may be subsequently detected using labeled antibodies (e.g., labeled sheep anti-human antibodies) that specifically bind to the anti-polypeptide.

In a more preferred embodiment, the polypeptide is detected using an immunoassay. As used herein, an immunoassay is an assay that utilizes an antibody to specifically bind to the analyte. The immunoassay is thus characterized by detection of specific binding of a polypeptide to an anti-antibody as opposed to the use of other physical or chemical properties to isolate, target, and quantify the analyte.

The polypeptide is detected and/or quantified using any of a number of well recognized immunological binding assays (see, e.g., U.S. Pat. Nos. 4,366,241; 4,376,110; 4,517,288; and 4,837,168). For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (polypeptide or subsequence). The capture agent is a moiety that specifically binds to the analyte. In a preferred embodiment, the capture agent is an antibody that specifically binds a polypeptide. The antibody (anti-peptide) may be produced by any of a number of means well known to those of skill in the art.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled anti-antibody. Alternatively, the labeling agent may be a third moiety, such as another antibody, that specifically binds to the antibody/polypeptide complex.

In one preferred embodiment, the labeling agent is a second human antibody bearing a label. Alternatively, the second antibody may lack a label, but it may, in turn, be bound by a labeled third antibody specific to antibodies of the species from which the second antibody is derived. The second can be modified with a detectable moiety, e.g., as biotin, to which a third labeled molecule can specifically bind, such as enzyme-labeled streptavidin.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

As indicated above, immunoassays for the detection and/or quantification of a polypeptide can take a wide variety of formats well known to those of skill in the art.

Preferred immunoassays for detecting a polypeptide are either competitive or noncompetitive. Noncompetitive immunoassays are assays in which the amount of captured analyte is directly measured. In one preferred “sandwich” assay, for example, the capture agent (anti-peptide antibodies) can be bound directly to a solid substrate where they are immobilized. These immobilized antibodies then capture polypeptide present in the test sample. The polypeptide thus immobilized is then bound by a labeling agent, such as a second human antibody bearing a label.

In competitive assays, the amount of analyte (polypeptide) present in the sample is measured indirectly by measuring the amount of an added (exogenous) analyte (polypeptide) displaced (or competed away) from a capture agent (anti-peptide antibody) by the analyte present in the sample. In one competitive assay, a known amount of, in this case, a polypeptide is added to the sample and the sample is then contacted with a capture agent. The amount of polypeptide bound to the antibody is inversely proportional to the concentration of polypeptide present in the sample.

In one particularly preferred embodiment, the antibody is immobilized on a solid substrate. The amount of polypeptide bound to the antibody may be determined either by measuring the amount of polypeptide present in a polypeptide/antibody complex, or alternatively by measuring the amount of remaining uncomplexed polypeptide. The amount of polypeptide may be detected by providing a labeled polypeptide.

The assays of this invention are scored (as positive or negative or quantity of polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of polypeptide.

Antibodies for use in the various immunoassays described herein, can be produced as described herein.

In another embodiment, level (activity) is assayed by measuring the enzymatic activity of the gene product. Methods of assaying the activity of an enzyme are well known to those of skill in the art.

In vivo techniques for detection of a biomarker protein include introducing into a subject a labeled antibody directed against the protein. For example, the antibody can be labeled with a radioactive biomarker whose presence and location in a subject can be detected by standard imaging techniques.

Certain biomarkers identified by the methods of the invention may be secreted proteins. It is a simple matter for the skilled artisan to determine whether any particular biomarker protein is a secreted protein. In order to make this determination, the biomarker protein is expressed in, for example, a mammalian cell, preferably a human cell line, extracellular fluid is collected, and the presence or absence of the protein in the extracellular fluid is assessed (e.g. using a labeled antibody which binds specifically with the protein).

The following is an example of a method which can be used to detect secretion of a protein. About 8×10⁵ 293T cells are incubated at 37° C. in wells containing growth medium (Dulbecco's modified Eagle's medium {DMEM} supplemented with 10% fetal bovine serum) under a 5% (v/v) CO2, 95% air atmosphere to about 60-70% confluence. The cells are then transfected using a standard transfection mixture comprising 2 micrograms of DNA comprising an expression vector encoding the protein and 10 microliters of LipofectAMINE™ (GIBCO/BRL Catalog no. 18342-012) per well. The transfection mixture is maintained for about 5 hours, and then replaced with fresh growth medium and maintained in an air atmosphere. Each well is gently rinsed twice with DMEM which does not contain methionine or cysteine (DMEM-MC; ICN Catalog no. 16-424-54).

About 1 milliliter of DMEM-MC and about 50 microcuries of Trans-³⁵S™ reagent (ICN Catalog no. 51006) are added to each well. The wells are maintained under the 5% CO₂ atmosphere described above and incubated at 37° C. for a selected period. Following incubation, 150 microliters of conditioned medium is removed and centrifuged to remove floating cells and debris. The presence of the protein in the supernatant is an indication that the protein is secreted.

It will be appreciated that subject samples, e.g., a sample containing tissue, whole blood, serum, plasma, buccal scrape, saliva, cerebrospinal fluid, urine, stool, bile, pancreatic juice or pancreatic tissue, may contain cells therein, particularly when the cells are cancerous, and, more particularly, when the cancer is metastasizing, and thus may be used in the methods of the present invention. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the level of expression of the biomarker in the sample. Thus, the compositions, kits, and methods of the invention can be used to detect expression of biomarkers corresponding to proteins having at least one portion which is displayed on the surface of cells which express it. It is a simple matter for the skilled artisan to determine whether the protein corresponding to any particular biomarker comprises a cell-surface protein. For example, immunological methods may be used to detect such proteins on whole cells, or well known computer-based sequence analysis methods (e.g. the SIGNALP program; Nielsen et al., 1997, Protein Engineering 10:1-6) may be used to predict the presence of at least one extracellular domain (i.e. including both secreted proteins and proteins having at least one cell-surface domain). Expression of a biomarker corresponding to a protein having at least one portion which is displayed on the surface of a cell which expresses it may be detected without necessarily lysing the cell (e.g. using a labeled antibody which binds specifically with a cell-surface domain of the protein).

The invention also encompasses kits for detecting the presence of a polypeptide or nucleic acid corresponding to a biomarker of the invention in a biological sample, e.g., a sample containing tissue, whole blood, serum, plasma, buccal scrape, saliva, urine, stool, bile, pancreatic cells or pancreatic tissue,. Such kits can be used to determine if a subject is suffering from or is at increased risk of developing cancer. For example, the kit can comprise a labeled compound or agent capable of detecting a polypeptide or an mRNA encoding a polypeptide corresponding to a biomarker of the invention in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

For antibody-based kits, the kit can comprise, for example: (1) a first antibody (e.g., attached to a solid support) which binds to a polypeptide corresponding to a biomarker of the invention; and, optionally, (2) a second, different antibody which binds to either the polypeptide or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a polypeptide corresponding to a biomarker of the invention or (2) a pair of primers useful for amplifying a nucleic acid molecule corresponding to a biomarker of the invention. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

4. Method for Detecting Structural Alterations

The invention also provides a method for assessing whether a subject is afflicted with cancer or is at risk for developing cancer by comparing the structural alterations, e.g., mutations or allelic variants, of a biomarker in a cancer sample with the structural alterations, e.g., mutations of a biomarker in a normal, e.g., control sample. The presence of a structural alteration, e.g., mutation or allelic variant in the biomarker in the cancer sample is an indication that the subject is afflicted with cancer.

A preferred detection method is allele specific hybridization using probes overlapping the polymorphic site and having about 5, 10, 20, 25, or 30 nucleotides around the polymorphic region. In a preferred embodiment of the invention, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix™). Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment. For example, the identity of the allelic variant of the nucleotide polymorphism in the 5′ upstream regulatory element can be determined in a single hybridization experiment.

In other detection methods, it is necessary to first amplify at least a portion of a biomarker prior to identifying the allelic variant. Amplification can be performed, e.g., by PCR and/or LCR (see Wu and Wallace (1989) Genomics 4:560), according to methods known in the art. In one embodiment, genomic DNA of a cell is exposed to two PCR primers and amplification for a number of cycles sufficient to produce the required amount of amplified DNA. In preferred embodiments, the primers are located between 150 and 350 base pairs apart.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al., (1988) Bio/Technology 6:1197), and self-sustained sequence replication (Guatelli et al., (1989) Proc. Nat. Acad. Sci. 87:1874), and nucleic acid based sequence amplification (NABSA), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, any of a variety of sequencing reactions known in the art can be used to directly sequence at least a portion of a biomarker and detect allelic variants, e.g., mutations, by comparing the sequence of the sample sequence with the corresponding reference (control) sequence. Exemplary sequencing reactions include those based on techniques developed by Maxam and Gilbert (Proc. Natl. Acad Sci USA (1977) 74:560) or Sanger (Sanger et al. (1977) Proc. Nat. Acad. Sci. 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Köster; U.S. Pat. No. 5,547,835 and international patent application Publication Number WO 94/21822 entitled DNA Sequencing by Mass Spectrometry Via Exonuclease Degradation by H. Köster), and U.S. Pat. No. 5,605,798 and International Patent Application No. PCT/US96/03651 entitled DNA Diagnostics Based on Mass Spectrometry by H. Köster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing.”

In some cases, the presence of a specific allele of a biomarker in DNA from a subject can be shown by restriction enzyme analysis. For example, a specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of a biomarker allelic variant with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, Cotton et al (1988) Proc. Natl Acad Sci USA 85:4397; Saleeba et al (1992) Methods Enzymol. 217:286-295. In a preferred embodiment, the control or sample nucleic acid is labeled for detection.

In another embodiment, an allelic variant can be identified by denaturing high-performance liquid chromatography (DHPLC) (Oefner and Underhill, (1995) Am. J. Human Gen. 57:Suppl. A266). DHPLC uses reverse-phase ion-pairing chromatography to detect the heteroduplexes that are generated during amplification of PCR fragments from individuals who are heterozygous at a particular nucleotide locus within that fragment (Oefner and Underhill (1995) Am. J. Human Gen. 57:Suppl. A266). In general, PCR products are produced using PCR primers flanking the DNA of interest. DHPLC analysis is carried out and the resulting chromatograms are analyzed to identify base pair alterations or deletions based on specific chromatographic profiles (see O'Donovan et al. (1998) Genomics 52:44-49).

In other embodiments, alterations in electrophoretic mobility are used to identify the type of biomarker allelic variant. For example, single strand conformation polymorphism (SSCP) may be used to detect differences in electrophoretic mobility between mutant and wild type nucleic acids (Orita et al. (1989) Proc Natl. Acad. Sci. USA 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence and the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the identity of an allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between two nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al (1989) Proc. Natl. Acad. Sci. USA 86:6230; and Wallace et al. (1979) Nucl. Acids Res. 6:3543). Such allele specific oligonucleotide hybridization techniques may be used for the simultaneous detection of several nucleotide changes in different polylmorphic regions of biomarker. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al (1992) Mol. Cell. Probes 6:1).

In another embodiment, identification of the allelic variant is carried out using an oligonucleotide ligation assay (OLA), as described, e.g., in U.S. Pat. No. 4,998,617 and in Landegren, U. et al., (1988) Science 241:1077-1080. The OLA protocol uses two oligonucleotides which are designed to be capable of hybridizing to abutting sequences of a single strand of a target. One of the oligonucleotides is linked to a separation biomarker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., (1990) Proc. Natl. Acad. Sci. (U.S.A.) 87:8923-8927. In this method, PCR is used to achieve the exponential amplification of target DNA, which is then detected using OLA.

The invention further provides methods for detecting single nucleotide polymorphisms in a biomarker. Because single nucleotide polymorphisms constitute sites of variation flanked by regions of invariant sequence, their analysis requires no more than the determination of the identity of the single nucleotide present at the site of variation and it is unnecessary to determine a complete gene sequence for each subject. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site (Cohen, D. et al. French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Several primer-guided nucleotide incorporation procedures for assaying polymorphic sites in DNA have been described (Komher, J. S. et al., (1989) Nucl. Acids. Res. 17:7779-7784; Sokolov, B. P., (1990) Nucl. Acids Res. 18:3671; Syvanen, A.-C., et al., (1990) Genomics 8:684-692; Kuppuswamy, M. N. et al., (1991) Proc. Natl. Acad. Sci. (U.S.A.) 88:1143-1147; Prezant, T. R. et al., (1992) Hum. Mutat. 1:159-164; Ugozzoli, L. et al., (1992) GATA 9:107-112; Nyren, P. (1993) et al., Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. C., et al., (1993) Amer. J. Hum. Genet. 52:46-59).

For determining the identity of the allelic variant of a polymorphic region located in the coding region of a biomarker, yet other methods than those described above can be used. For example, identification of an allelic variant which encodes a mutated biomarker can be performed by using an antibody specifically recognizing the mutant protein in, e.g., immunohistochemistry or immunoprecipitation. Antibodies to wild-type biomarker or mutated forms of biomarkers can be prepared according to methods known in the art.

Alternatively, one can also measure an activity of a biomarker, such as binding to a biomarker ligand. Binding assays are known in the art and involve, e.g., obtaining cells from a subject, and performing binding experiments with a labeled ligand, to determine whether binding to the mutated form of the protein differs from binding to the wild-type of the protein.

B. Pharmacogenomics

Agents or modulators which have a stimulatory or inhibitory effect on amount and/or activity of a biomarker of the invention can be administered to individuals to treat (prophylactically or therapeutically) cancer in the subject. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the amount, structure, and/or activity of the invention in an individual can be determined to thereby select appropriate agent(s) for therapeutic or prophylactic treatment of the individual.

Pharmacogenomics deals with clinically significant variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some subjects do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the amount, structure, and/or activity of a biomarker of the invention in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of amount, structure, and/or activity of a biomarker of the invention.

C. Monitoring Clinical Trials

Monitoring the influence of agents (e.g., drug compounds) on amount, structure, and/or activity of a biomarker of the invention can be applied not only in basic drug screening, but also in clinical trials. For example, the effectiveness of an agent to affect biomarker amount, structure, and/or activity can be monitored in clinical trials of subjects receiving treatment for cancer. In a preferred embodiment, the present invention provides a method for monitoring the effectiveness of treatment of a subject with an agent (e.g., an agonist, antagonist, peptidomimetic, protein, peptide, antibody, nucleic acid, antisense nucleic acid, ribozyme, small molecule, RNA interfering agent, or other drug candidate) comprising the steps of (i) obtaining a pre-administration sample from a subject prior to administration of the agent; (ii) detecting the amount, structure, and/or activity of one or more selected biomarkers of the invention in the pre-administration sample; (iii) obtaining one or more post-administration samples from the subject; (iv) detecting the amount, structure, and/or activity of the biomarker(s) in the post-administration samples; (v) comparing the amount, structure, and/or activity of the biomarker(s) in the pre-administration sample with the amount, structure, and/or activity of the biomarker(s) in the post-administration sample or samples; and (vi) altering the administration of the agent to the subject accordingly. For example, increased administration of the agent can be desirable to increase amount and/or activity of the biomarker(s) to higher levels than detected, i.e., to increase the effectiveness of the agent. Alternatively, decreased administration of the agent can be desirable to decrease amount and/or activity of the biomarker(s) to lower levels than detected, i.e., to decrease the effectiveness of the agent.

D. Surrogate Markers

The biomarkers of the invention may serve as surrogate markers for one or more disorders or disease states or for conditions leading up to disease states, and in particular, pancreatic cancer, e.g., pancreatic adenocarcinoma. As used herein, a “surrogate biomarker” is an objective biochemical biomarker which correlates with the absence or presence of a disease or disorder, or with the progression of a disease or disorder (e.g., with the presence or absence of a tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate biomarker, and an analysis of HIV infection may be made using HIV RNA levels as a surrogate biomarker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The biomarkers of the invention are also useful as pharmacodynamic markers. As used herein, a “pharmacodynamic biomarker” is an objective biochemical biomarker which correlates specifically with drug effects. The presence or quantity of a pharmacodynamic biomarker is not related to the disease state or disorder for which the drug is being administered; therefore, the presence or quantity of the biomarker is indicative of the presence or activity of the drug in a subject. For example, a pharmacodynamic biomarker may be indicative of the concentration of the drug in a biological tissue, in that the biomarker is either expressed or transcribed or not expressed or transcribed in that tissue in relationship to the level of the drug. In this fashion, the distribution or uptake of the drug may be monitored by the pharmacodynamic biomarker. Similarly, the presence or quantity of the pharmacodynamic biomarker may be related to the presence or quantity of the metabolic product of a drug, such that the presence or quantity of the biomarker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of biomarker transcription or expression, the amplified biomarker may be in a quantity which is more readily detectable than the drug itself. Also, the biomarker may be more easily detected due to the nature of the biomarker itself; for example, using the methods described herein, antibodies may be employed in an immune-based detection system for a protein biomarker, or biomarker-specific radiolabeled probes may be used to detect a mRNA biomarker. Furthermore, the use of a pharmacodynamic biomarker may offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J Health-Syst. Pharm. 56 Suppl. 3: S16-S20.

### EXAMPLES

This invention is further illustrated by the following examples which should not be construed as limiting. The contents of all references, figures, Sequence Listing, patents and published patent applications cited throughout this application are hereby incorporated by reference.

### Example 1

### Generation of a Mouse Model of Pancreatic Adenocarcinoma

### A. Materials and Methods

Engineering of the Conditional Ink4a/Arf Mouse Strain

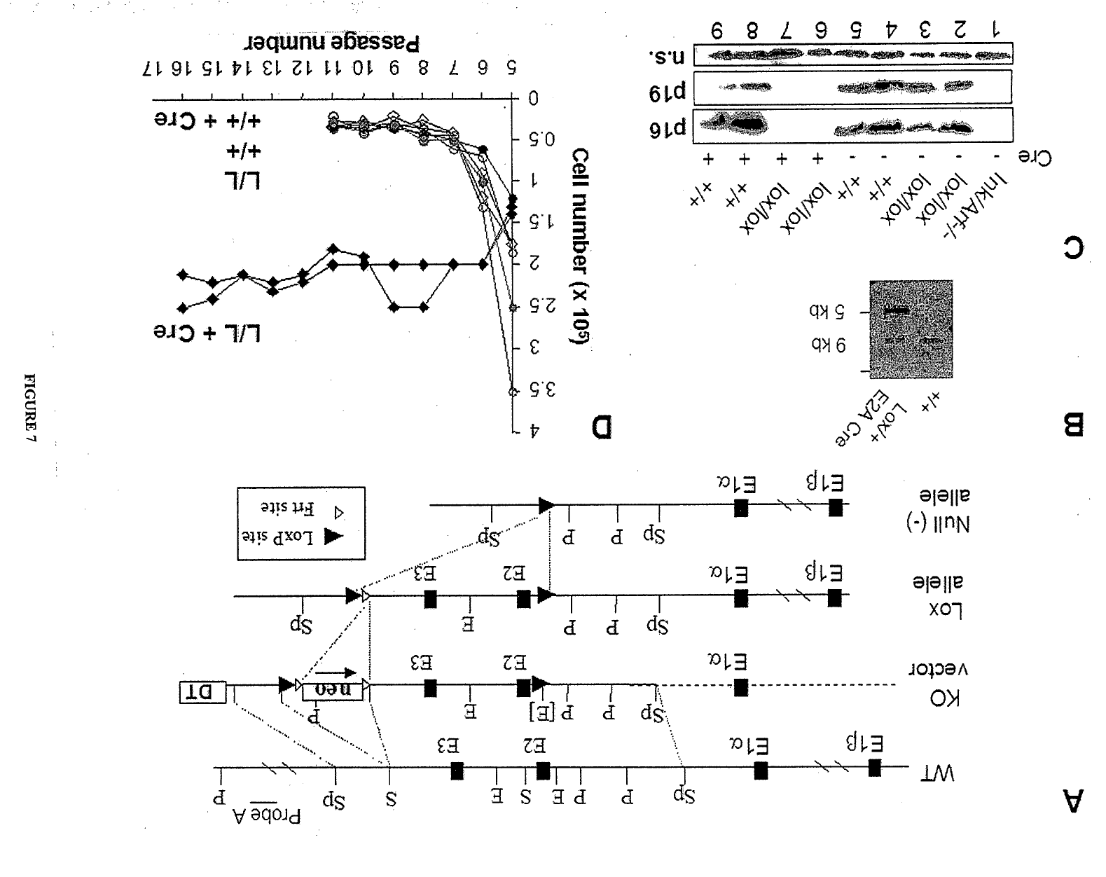

The Ink4a/Arf locus was subcloned into the pKOII targeting vector that carried a negative selection marker for diptheria toxin (DT), a positive selection marker for neomycin acetyltransferase (Neo), Frt sites and loxP sites (FIG. 7). Embryonic stem (ES) cells were electroporated and selected by standard techniques. Clones were screened by Southern analysis using the PstI restriction enzyme and a 3′ fragment external to the targeting construct (FIG. 7). Blastocyst injections were carried out with targeted clones, and transmitting chimeric mice were bred to CAGG-Flpe and transgenic mice to generate the Ink4a/Arf lox allele. These mice were bred onto an FVB/n background (backcrossed 4 generations). Mice were genotyped by Southern analysis and multiplex PCR (primers and conditions are available on request). For functional tests of the allele, these mice were crossed to the EIIA-Cre general deleter strain (Lakso, M., J et al. (1996) Proc Natl Acad Sci USA 93: 5860-5) resulting in efficient deletion of exons 2 and 3 as assessed by Southern blot. Methods for testing the functionality of the Ink4a/Arf^{lox} allele in mouse embryonic fibroblasts (MEFs), including MEF isolation and culturing, Cre-mediated deletion and 3T3 assay, were performed as described (Bardeesy, N., et al. (2002b) Nature 419: 162-7).

### Mouse Colony Generation

The LSL-Kras^{G12D} knock-in strain (Jackson, E. L. et al. (2001) Genes Dev 15: 3243-8) and the Pdx1-Cre transgenic strain (Gu, G., et al. (2002) Development 129: 2447-57) were bred to Ink4a/Arf^{lox/lox} mice to generate the genotypes, Pdx1-Cre; Ink4a/Arf^{lox/+} and LSL-Kras^{G12D}; Ink4a/Arf^{lox/lox}. These strains were intercrossed to produce the experimental cohorts. Mice were genotyped by slot blot and PCR.

### Histology and Immunohistochemistry

Tissues were fixed in 10% formalin overnight and embedded in paraffin. For immunohistochemistry, slides were deparaffinized in xylene and rehydrated sequentially in ethanol. For antibodies requiring antigen retrieval, Antigen Unmasking Solution (Vector) was used according to the manufacturer's instructions. Slides were quenched in hydrogen peroxide (0.3-3%) to block endogenous peroxidase activity and then washed in Automation Buffer (Biomeda). Slides were blocked in 5% normal serum for 1 hour at room temperature. Slides were incubated at 4° C. overnight with primary antibody diluted in blocking buffer. The avidin-biotin peroxidase complex method (Vector) was used and slides were counterstained with hematoxylin. Slides were dehydrated sequentially in ethanol, cleared with xylenes and mounted with Permount (Fisher). The antibodies and dilutions were amylase, 1:500 (Calbiochem), insulin 1:100 (Dako), TROMA3 (cytokeratin 19) 1:10, and EGFR, 1:50 (Cell Signaling). EGFR and cytokeratin staining required antigen unmasking. Biotinylated DBA lectin (Vector) was used at 1:100.

### Establishment and Cultivation of Primary Pancreatic Adenocarcinoma Cell Lines

Freshly isolated tumor specimens were minced with sterile razor blades, digested with dispase II/colagenase (4 mg/ml each) for 1 hour at 37° C. and then resuspended in RPMI+20% fetal calf serum and seeded on vitrogen/fibronectin coated plates. Cells were passaged by trypsinization. All studies were done on cells cultivated fewer than 7 passages.

### Molecular Analyses

RNA was isolated by the Trizol reagent (Gibco), treated with RQ1 DNAse (Promega), and then purified by the RNAeasy kit (Qiagen), each according to manufacturer's instructions. RNA was reverse-transcribed using Superscript II (Gibco). PCR primers were designed to amplify the entire Smad4 and p53 coding regions as a series of overlapping 400-500 bp fragments.

For Smad4 the primer pairs were:

| | | |
|---|---|---|
| A, | 5′-TCCAGAAATTGGAGAGTTGGA-3′, | (SEQ ID NO.:2) |
| A1, | 5′-TCAATTCCAGGTGAGACAACC-3′, | (SEQ ID NO.:3) |
| B, | 5′-TGACAGTGTCTGTGTGAATCCAT-3′, | (SEQ ID NO.:4) |
| B1, | 5′-TTAGGTGTGTATGGTGCAGTCC-3′, | (SEQ ID NO.:5) |
| C, | 5′-ACAGCACTACCACCTGGACTGG-3′, | (SEQ ID NO.:6) |
| C1, | 5′-ACAAAGACCGCGTGGTCACTAA-3′, | (SEQ ID NO.:7) |
| D, | 5′-TTTGGGTCAGGTGCCTTAGTGA-3′, | (SEQ ID NO.:8) |
| D1, | 5′-GTCCACCATCCTGGAAATGGT-′3. | (SEQ ID NO.:9) |

For p53 the primer pairs were:

| | | |
|---|---|---|
| E, | 5′-GTGTCACGCTTCTCCGAAGACT-3′, | (SEQ ID NO.:10) |
| E1, | 5′-CGTCATGTGCTGTGACTTCTTGT-3′, | (SEQ ID NO.:11) |
| F, | 5′-GCACGTACTCTCCTCCCCTCAA-3′, | (SEQ ID NO.:12) |
| F1, | 5′-AGGCACAAACACGAACCTCAAA-3′ | (SEQ ID NO.:13) |
| G, | 5′-ATGAACCGCCGACCTATCCTTA-3′, | (SEQ ID NO.:14) |
| G1, | 5′-GGATTGTGCTCAGCCCTGAAGT-3′. | (SEQ ID NO.:15) |

PCR products were subjected to direct sequencing with one of the primers used in the PCR. RT-PCR/RFLP analysis to distinguish the wild-type Kras and Kras^{G12D} mutant transcripts utilized primers:

| | | |
|---|---|---|
| KRAS1: | 5′-AGGCCTGCTGAAAATGACTG-3′ | (SEQ ID NO.:16) |
| and | | |
| KRAS7: | 5′-CCCTCCCCAGTTCTCATGTA-3′ | (SEQ ID NO.:17) |

to amplify a 243 bp product from both the wild-type and mutant transcripts. The Kras^{G12D} allele but not the wild-type allele contains a HindIII restriction site engineered in exon 1. Thus, digestion of the 243 bp PCR product with HindIII yields 213 bp and 30 bp bands from the mutant product only. For Western blot analyses, tissues or cell pellets were sonicated in 20 mM Tris (pH 7.5), 150 mM NaCl, 1 mM EDTA, 1 mM EGTA, 1% Triton X-100 in the presence of a protease inhibitor cocktail (Roche) and a phosphatase inhibitor cocktail (kits I and II, Calbiochem). 50 μg of lysate was resolved on 4-12% Bis-Tris NuPAGE gels (Invitrogen) and transferred to PVDF membranes. Membranes were blotted for p16 Ink4a (M-156, Santa Cruz), p53 (Ab-7, Oncogene Research), Smad4 (B-8, Santa Cruz), p21 (C-19, Santa Cruz), p19 Arf (Ab-80, Abcam), and tubulin (DM-1A, Sigma). For p53 induction, cells were irradiated using a cesium source (Atomic Energy Commission of Canada) at 10 Gy and harvested after 4 hrs. For measurement of activated Ras level, the Ras activation assay kit (Upsate) was used according to the manufacturer's instructions.

### Quantitative Real-Time PCR

PCR primers were designed to amplify a 154 bp product of Kras genomic DNA encompassing exon 1

(SEQ ID NO.:18)

| | |
|---|---|
| KrasE1-F: | 5′-TGTAAGGCCTGCTGAAAATG-3′, |

(SEQ ID NO.:19)

| | |
|---|---|
| KrasE1-R: | 5′-GCACGCAGACTGTAGAGCAG-3′. |

Quantitative PCR was performed by monitoring in real-time the increase in fluorescence of SYBR Green dye (Qiagen) with an ABI 7700 sequence detection system (Perkin Elmer Life Sciences). Data was analyzed by relative quantitation using the comparative Ct method and normalization to GAPDH.

### B. Results

Generation of Mouse Strains with Pancreas-Specific Kras^{G12D} Expression and Ink4a/Arf Deletion

To model the unique and cooperative interactions of two signature genetic lesions encountered in human pancreatic adenocarcinoma, mouse strains harboring a latent Kras^{G12D} knock-in allele (LSL-Kras) (Jackson, E. L. et al. (2001) Genes Dev 15: 3243-8) and/or a conditional Ink4a/Arf knockout allele in the germline were characterized. As described previously (Jackson, E. L. et al. (2001) Genes Dev 15: 3243-8), the LSL-Kras^{G12D} allele is expressed at endogenous levels following Cre-mediated excision of a transcriptional stopper element and may aide in the directed expression of Kras^{G12D} in the appropriate cell-of-origin for a given cancer type. The conditional Ink4a/Arf allele (Ink4a/Arf^{lox}) was engineered to sustain Cre-mediated excision of exons 2 and 3, thereby eliminating both p16INK4A and p19ARF proteins (FIG. 7). The performance of this allele was assessed genetically and functionally: 1) Crosses of Ink4a/Arf^{lox/lox} mice to the EIIA-Cre constitutive deleter strain produced offspring with the expected deletion product, 2) Ink4a/Arf^{lox/lox} mouse embryonic fibroblasts showed normal levels of p16^{INK4A} and p19^{ARF} and underwent passage-induced senescence with similar kinetics to wild-type cells, whereas infection of these cells with retroviruses encoding Cre recombinase caused extinction of expression of both p16^{INK4A} and p19^{ARF} and resulted in immortal cell growth, 3) Ink4a/Arf^{lox/lox} mice showed similar tumor incidences and life spans to wildtype mice (FIG. 7). These data indicate that the Ink4a/Arf^{lox} allele retains wild-type function and can be rendered null for both Ink4a and Arf by Cre recombinase activity. To express the Kras^{G12D} allele and to delete both copies of the conditional Ink4a/Arf allele specifically in the pancreas, the Pdx1-Cre transgene, shown previously to delete efficiently loxP-containing alleles in all pancreatic lineages (endocrine, exocrine and duct cells) (Gu, G., et al. (2002) Development 129: 2447-57) was utilized.

### Kras^{G12D} Provokes Premalignant Ductal Lesions

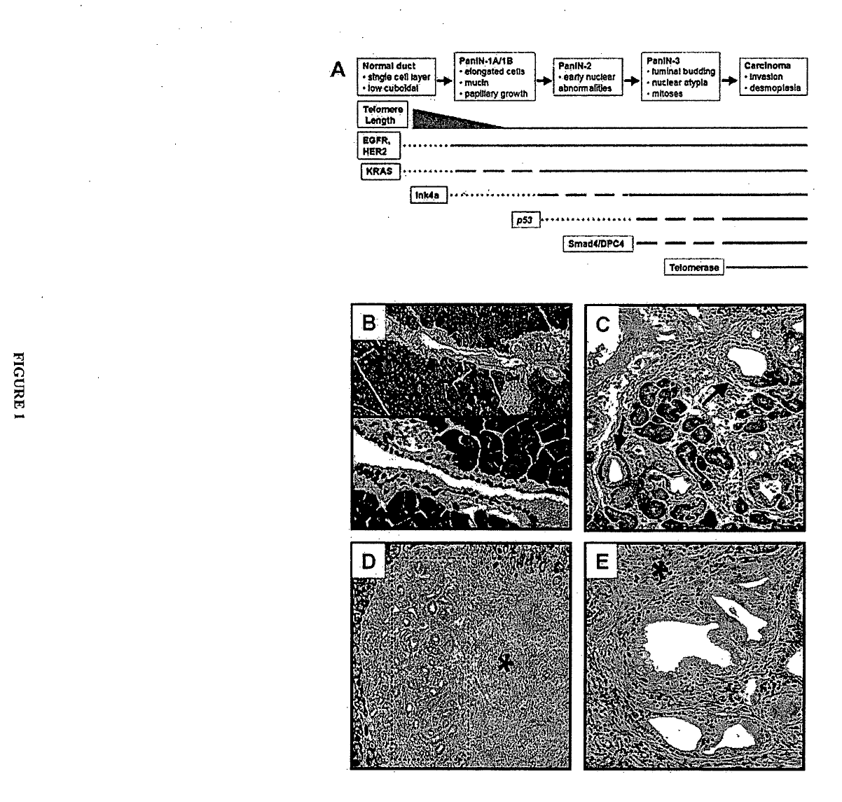

Histolopathologic and genetic analyses of human specimens have generated a staged progression model for premalignant and malignant pancreatic ductal lesions and their corresponding mutational profile (FIG. 1a). To examine the role of Kras^{G12D} on the initiation and progression of pancreatic neoplasia, a cohort of Pdx1-Cre; LSL-Kras^{G12D} mice was generated and assessed for pancreatic pathology. Compound Pdx1-Cre and LSL-Kras^{G12D} strains were born at the expected Mendelian ratios and without evidence of clinical compromise. Up to an age of 30 weeks (n=15), Pdx1-Cre; LSL-Kras^{G12D} mice showed no outward signs of ill-health. Correspondingly, serum glucose, amylase, lipase, albumin, and total bilirubin measurements in these mice (n=5) were within normal limits which indicates normal pancreatic structure and physiology. Furthermore, histologically normal islets, acini and ducts were clearly evident in the pancreata of these mice at all stages analyzed (FIG. 1b). Serial histological surveys (9, 12, 18 and 26 weeks) however, revealed pancreatic ductal lesions strongly reminiscent of human PanINs. In younger mice, only small PanIN-1 lesions consisting of elongated, mucinous ductal cells were detected (FIG. 1c). By 12 weeks, larger and more proliferative ductal lesions were noted (FIG. 1d) and these changes became more pronounced with age. At 26 weeks, extensive regions of the pancreatic parenchyma had been replaced by PanINs, surrounded by a pronounced fibrous stroma (FIG. 1e). Although PanINs increased in number and size with age, no invasive tumors were seen up to 30 weeks of age in any of the 15 mice analyzed. While there was no evidence of neoplasia in the acinar cell compartment, focal reactive metaplastic changes were observed—likely relating to regional ductal obstruction by PanINs. Similarly, some pancreatic islets appeared moderately enlarged yet showed no evidence of neoplasia. The modest impact of Kras^{G12D} expression on these compartments is concordant with the normal weight gains and serum chemistries noted above. Mice harboring either the LSL-Kras^{G12D} or the Pdx1-Cre alleles alone showed no pancreatic abnormalities. These results are in line with an extensive independent study of mice harboring LSL-Kras^{G12D} and either a different Pdx1-Cre allele or a p48-Cre allele (Kawaguchi, Y., et al. (2002) Nat Genet. 32: 128-34). In this study both compound strains displayed progressive premalignant lesions with ductal histology, an observation consistent with an initiating role for activated KRAS in pancreatic adenocarcinoma.

Ink4a/Arf Loss Causes Malignant Progression in the Pancreas

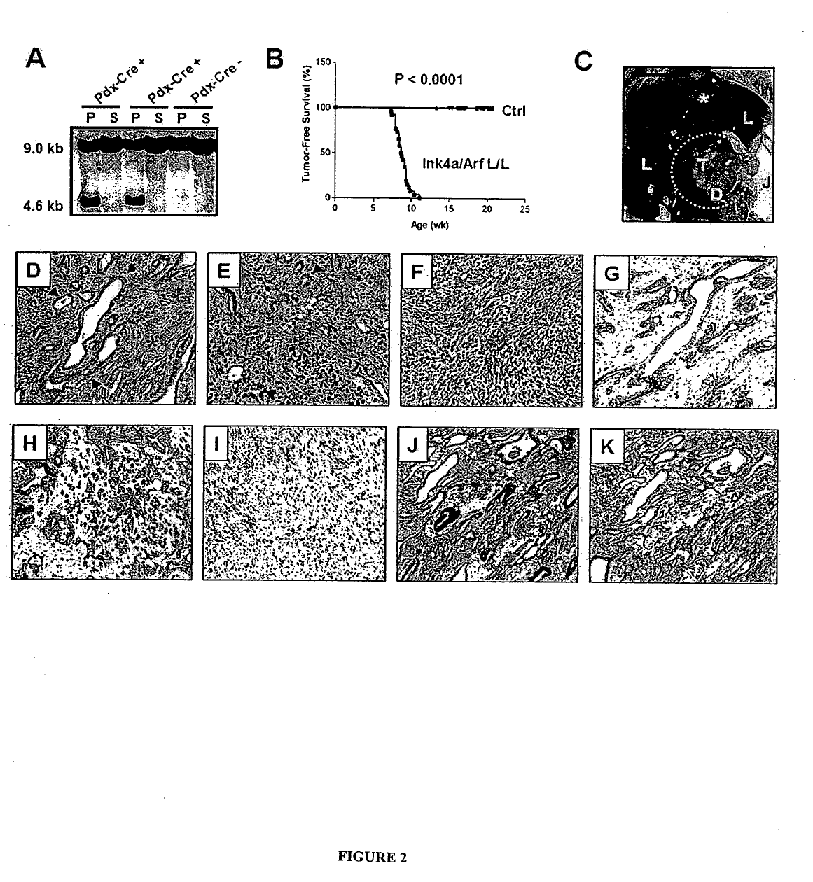

The Ink4a/Arf locus has been proposed to restrain the oncogenic potential of activated Ras genes, a concept in line with the high degree of coincident mutations of these genes in human cancers and the role of Ink4a/Arf loss in facilitating the oncogenicity of activated Hras in vitro and in vivo (Serrano, M., et al. (1996) Cell 85: 27-37; Chin, L., J. et al. (1997) Genes Dev 11: 2822-34; Kamijo, T., et al. (1997) Cell 91: 649-59; Rozenblum, E., M. et al. (1997) Cancer Res 57: 1731-4; Serrano, M., et al. (1997) Cell 88: 593-602; Ruas, M. and G. Peters (1998) Biochim Biophys Acta 1378: F115-77). The lack of progression of the PanIN lesions in Pdx1-Cre; LSL-Kras^{G12D} mice prompted an assessment of the combined impact of Kras^{G12D} expression and Ink4a/Arf deletion in the pancreas. Southern blots of whole pancreas DNA from Pdx1-Cre; Ink4a/Arf^{lox/+} mice revealed efficient deletion of the Ink4a/Arf^{lox} allele (FIG. 2a). Up to an age of 7 weeks, mice of each genotype were clinically normal, however between 7 and 11 weeks of age all Pdx1-Cre; LSL-Kras^{G12D}; Ink4a/Arf^{lox/lox} animals (n=26) became moribund (see FIG. 2b, survival curve), commonly presenting with weight loss, ascites, jaundice (FIG. 2c), and a palpable abdominal mass (Table 1). Autopsies revealed the presence of solid pancreatic tumors ranging in diameter from 4 to 20 mm (FIG. 2c, Table 1). Grossly, these tumors were firm with irregular and ill-defined margins, frequently adhering to adjacent organs and to the retroperitoneum. In some cases, more than one distinct tumor nodule was apparent, indicating that these neoplasms may be multi-focal in nature. The tumors were highly invasive, frequently involving the duodenum and/or spleen and occasionally obstructing the common bile duct (Table 1). Liver and lung metastases were not grossly evident. No tumors were observed in control cohorts, including the Pdx1-Cre; LSL-Kras^{G12D}; Ink4a/Arf^{lox/+} and Pdx1-Cre; Ink4a/Arf^{lox/lox} animals, up to an age of 21 weeks. The absence of pancreatic cancers in mice lacking Ink4a/Arf in the pancreas, together with the powerful synergy of this lesion in promoting the advancement of Kras^{G12D} induced PanINs, points to a role of Ink4a/Arf in constraining the malignant progression of early stage ductal neoplasms rather than in regulating the initiating phases of tumorigenesis.

TABLE 1

| | Ag<< | | | | Bilaiy | | Bloody | |
|---|---|---|---|---|---|---|---|---|
| ID | (wk) | Size (cm) | Location | *][£·** | Obstruct | Jaundice | Ascites | InvationiMetastaste (m) |
| 30 | 7.9 | 1 | Head | Y | N | N | Y | D, C.S, A |
| 31 | 7.9 | 1.4 | Head | N | Y | Y | Y | D. S |
| 32 | 7.9 | 2 | Head | Y | Y | K | N | D.PN.L, S, RN (m) |
| 33 | 7.9 | 1.4 | Tail | Y | N | N | Y | SP, RP, DP |
| 35 | 7.4 | 0.5 | Body | Y | N | N | N | V |
| 43 | 8.4 | >2 | Entire panc | Y | N | N | Y | D, S |
| 44 | 7.3 | 2 | Entire panc | N | N | N | N | V |
| 45 | 8.6 | 1 | Head | Y | N | N | Y | SP, L.E.S, D |
| 46 | 8.9 | 0.6 | Head | Y | Y | Y | N | D |
| 52 | 8.1 | 1 | Head | N | N | N | N | — |
| 58 | 8.7 | 0.5 | Head | Y | N | N | N | V. PN |
| 59 | 9.3 | 1.5 | Entire panc | Y | Y | Y | N | RP, RN |
| 60 | 9.9 | 0\hat{ }9 | Head | N | Y | Y | Y | D, PN, V, L(m). RP, A, DP |
| 61 | 9.3 | N/A | Head | Y | Y | Y | N/A | PN, L(m), D |
| 62 | 10.4 | 1.5 | Head | Y | Y | N | Y | PN.S.D.C |
| 63 | 9.3 | N/A | Entire panc | Y | Y | N | Y | GB, L (m), V, PN, S, D |
| 64 | 9.1 | 1.5 | Entire panc | Y | Y | N | Y | S. C. PN, SP |
| 65 | 9.7 | 0.4 | Head | Y | Y | Y | N | PN (m). D |
| 76 | 11.1 | 0.75 | Head | N | N | N | N | SP, D |
| 81 | 8.6 | 1.5 | Head | Y | N | N | Y | V, LN, |
| 90 | 8.4 | 2 | Entire panc | N | N | N | N | PN, D |
| 91 | 8.3 | 1 | Head | N | N | N | N | D. RN |
| 94 | 8.3 | N/A | Inferior Panc | Y | NA | MA | N/A | LN. D |
| 97 | 9.4 | 1 | Tail | N | N | N | N | LN |

Table 1: Data from clinically compromised Pdx1-Cre LSL-KRAS cInk4a/Arf^{lox/lox} animals.
Tumor size is given as the approximate diameter of largest distinct tumor nodule when multiple foci were present. Tumors were often large and invasive, making size approximation difficult.
Location indicates relative relation of the pancreatic tumor to the adjacent organs, with head, body and tail used in reference to the human anatomy.
“Inferior Pane” refers to the location of mouse pancreatic tissue affixed to the intestines, for which there is no analogous human pancreatic tissue.
Weight Loss was judged by signs of physical wasting and decreased weight compared with littermates.
Jaundice and bloody ascites were clinical observations made on autopsy.
Invasion/metastasis are noted by location as follows: duodenum (D), stomach (S), colon (C), peripancreatic lymph node (PN), renal lymph node (RN), adrenal gland (A), liver (L), spleen (SP), retroperitoneum (RP), diaphragm (DP), venous (V), esophagus (E), unspecified lymph node (LN), gall bladder (GB). Metastases are indicated by (m) following the indicated location.

### p53 Accelerates Malignant Progression in the Pancreas

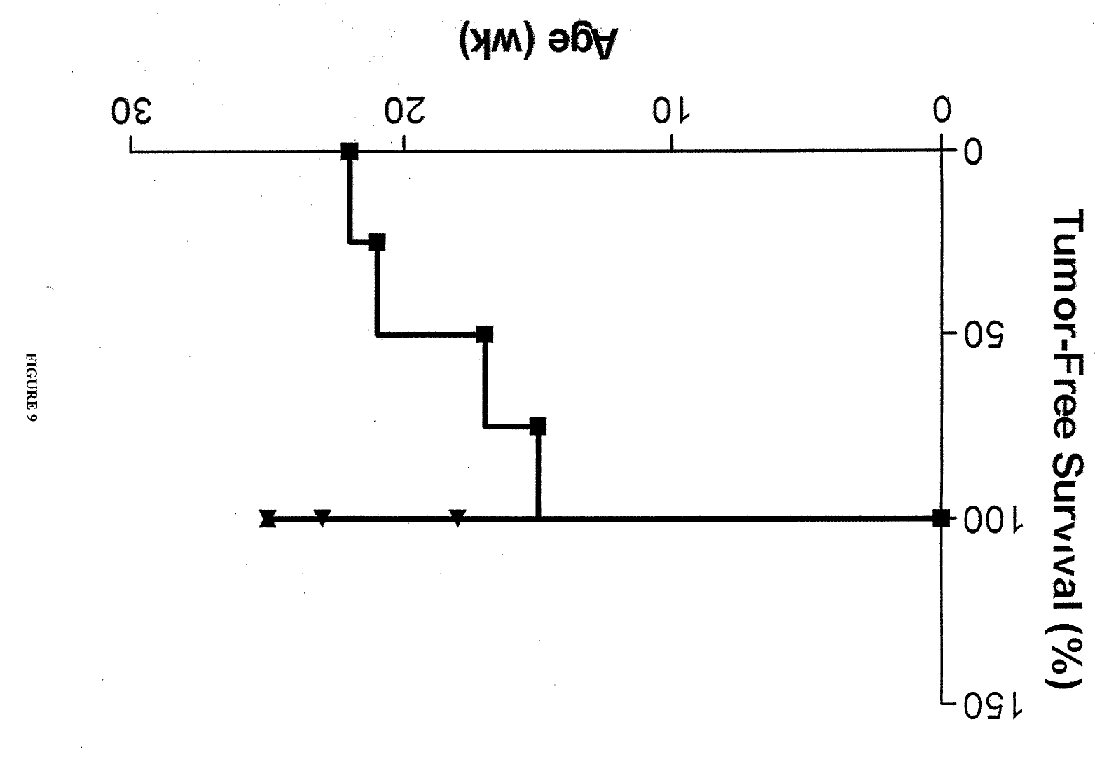

Mice carrying Pdx1-Cre; LSL-Kras^{G12D}; Ink4a^{lox/+}; p53^{lox/+} became moribund (see FIG. 9, survival curve), more rapidly than animals carrying Pdx1-Cre; LSL-Kras^{G12D}; Ink4a/Arf^{lox/lox} (see FIG. 2b) and commonly present with weight loss, ascites, jaundice, and a palpable abdominal mass.

### Murine Pancreatic Tumors Recapitulate the Pathologic Features of Human Pancreatic Adenocarcinoma

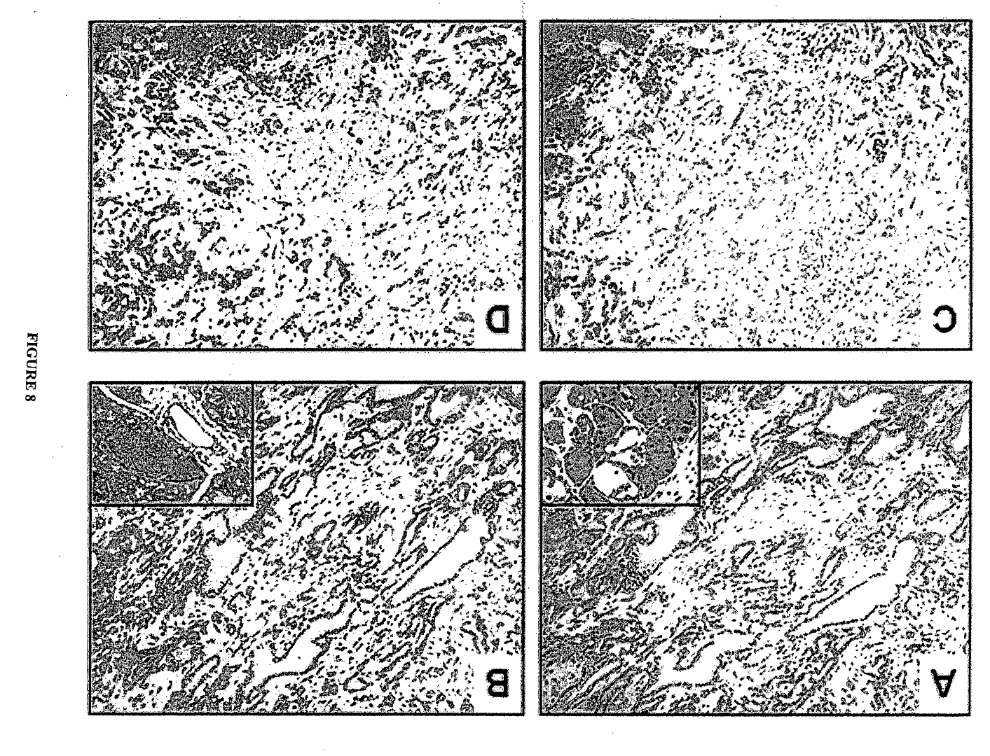

Microscopic examination of the tumors revealed a well-formed glandular morphology with resemblance to well-differentiated and moderately-differentiated human pancreatic ductal adenocarcinomas (FIG. 2d). Human pancreatic adenocarcinomas predominantly express ductal markers and lack expression of exocrine and endocrine markers (Solcia, E., et al. (1995) Tumors of the Pancreas. Armed Forces Institute for Pathology, Washington, D.C.). Consistent with a ductal phenotype, the glandular components of the murine tumors were positive for cytokeratin (Ck)-19 immunohistochemistry (FIGS. 2g and h), reacted with DBA lectin (data not shown), and showed mucin content by periodic acid Schiff plus diastase (PAS+D) (FIG. 2j) and alcian blue staining. Furthermore, the tumors showed stromal collagen deposition as evidenced by positive trichrome staining (FIG. 2k). None of the tumors showed reactivity for amylase and insulin, markers for acinar and β-cell differentiation, respectively (FIG. 8). The histological and immunohistochemical profiles of these experimental tumors bear striking resemblance to human pancreatic ductal adenocarcinoma.

In addition to well- and moderately differentiated ductal adenocarcinoma, regions of undifferentiated/anaplastic (sarcomatoid) tumor (FIGS. 2e and f) were observed in most cases. These anaplastic regions show weak or patchy reactivity with anti-Ck-19 antibodies and the DBA lectin (FIG. 2h, 2i). These tumors exhibited high mitotic activity, severe nuclear atypia and extensive cellular pleomorphism. In some tumors, several grades of differentiation ranging from well-differentiated to anaplastic carcinoma were noted. The epithelial nature of these anaplastic regions was confirmed by electron microscopy that revealed the presence of intermembranous junctions and microvilli.

### Invasion and Metastasis of Murine Pancreatic Adenocarcinomas

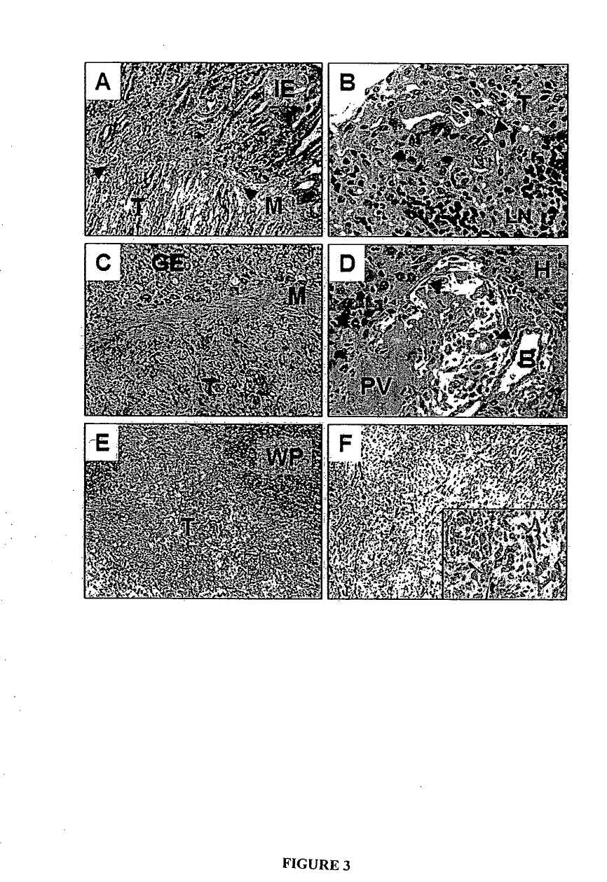

Local invasion and metastatic spread are pathologic hallmarks of advanced human pancreatic adenocarcinoma. Pancreatic tumors arising in Pdx1-Cre; LSL-Kras^{G12D}; Ink4a/Arf^{lox/lox} animals showed extensive invasion of adjacent organs including the duodenum, stomach, liver and spleen (Table 1, FIGS. 3a, c and e). Tumor encroachment of the retroperitoneum and diaphragm was also observed. Furthermore, invasion of the lymphatic and vascular system was frequently detected, an observation suggestive of metastatic potential of these neoplasms. Notably, invasion by both the glandular and anaplastic components of the tumors was observed, and invading tumor cells were shown to stain positively for Ck-19 (FIG. 3f).

Given the invasive nature of these lesions, a systematic histologic survey of distant organs in a subset of cases was conducted. This survey revealed metastases to the lymph nodes (FIG. 3b) and occasionally to the liver (FIG. 3d), although lung metastases were not observed. Metastases were often multi-focal in nature but small in size. The extensive invasion of the vasculature and lymph nodes detected in these mice make it likely that the histologic survey underestimates the true metastatic nature of these tumors. Overall, the distribution pattern of metastases is similar to that observed in the human disease which most commonly spreads to the liver and regional lymph nodes (Solcia, E., et al. (1995) Tumors of the Pancreas. Armed Forces Institute for Pathology, Washington, D.C.).

Accelerated Malignant Transformation in the Pdx1-Cre; LSL-Kras; Ink4a/Arf^{lox/lox} Pancreas

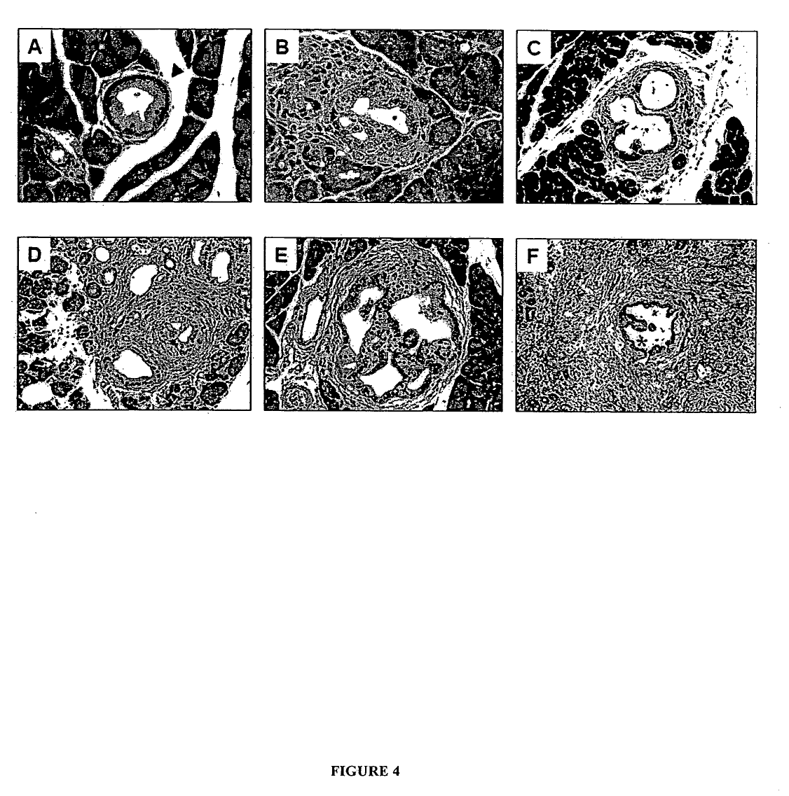

To understand better the earlier stages of tumor development produced by KRAS activation and homozygous Ink4a/Arf deficiency, an autopsy series was performed on outwardly normal Pdx1-Cre; LSL-Kras; Ink4a/Arf^{lox/lox} and Pdx1-Cre; LSL-Kras; Ink4a/Arf^{lox/+} littermates at ages 3, 4, 5 and 6 weeks. At all ages, the Pdx1-Cre; LSL-Kras^{G12D}; Ink4a/Arf^{lox/+} animals exhibited normal gross pancreatic histology with rare and isolated PanIN-1a lesions. At 3 weeks, Pdx1-Cre; LSL-Kras; Ink4a/Arf^{lox/lox} animals had primarily normal pancreatic histology; however, low-grade ductal lesions were also observed in all mice at this timepoint (FIG. 4b, n=6 mice). In addition to low-grade PanIN lesions, two of these mice also showed occasional foci of malignant ductal cells. By 4 weeks, these mice (n=4) had an increased overall number and higher grade of pancreatic ductal lesions relative to littermates (FIG. 4c). Furthermore, early-stage adenocarcinomas were detected at this age and importantly, these tumors showed both ductal and anaplastic morphologies from their earliest inception (FIG. 4d). At five weeks, most mice exhibited small multifocal pancreatic adenocarcinoma although exhaustive serial sectioning revealed that some animals had only advanced PanINs and had not yet progressed to frank malignancy (FIG. 4e). Notably, in several cases advanced PanIN lesions could be found surrounded by invasive ductal and anaplastic tumor cells (FIG. 4f), an observation consistent with these tumors arising from the progression of such PanIN lesions. By six weeks of age, all Pdx1-Cre; LSL-Kras^{G12D}; Ink4a/Arf^{lox/lox} mice analyzed had small pancreatic adenocarcinomas (n=5) with histology resembling the invasive tumors arising in adult mice. Thus, it appears that once initiated, the pancreatic ductal lesions in Pdx1-Cre; LSL-Kras^{G12D}; Ink4a/Arf^{lox/lox} mice undergo rapid histologic and clinical progression to invasive pancreatic adenocarcinoma. These features are supportive of the model in which human ductal adenocarcinoma derives from PanIN lesions with activating KRAS mutations that progress towards malignancy upon loss of INK4A/ARF function.

### Molecular Analyses of Pancreatic Adenocarcinomas

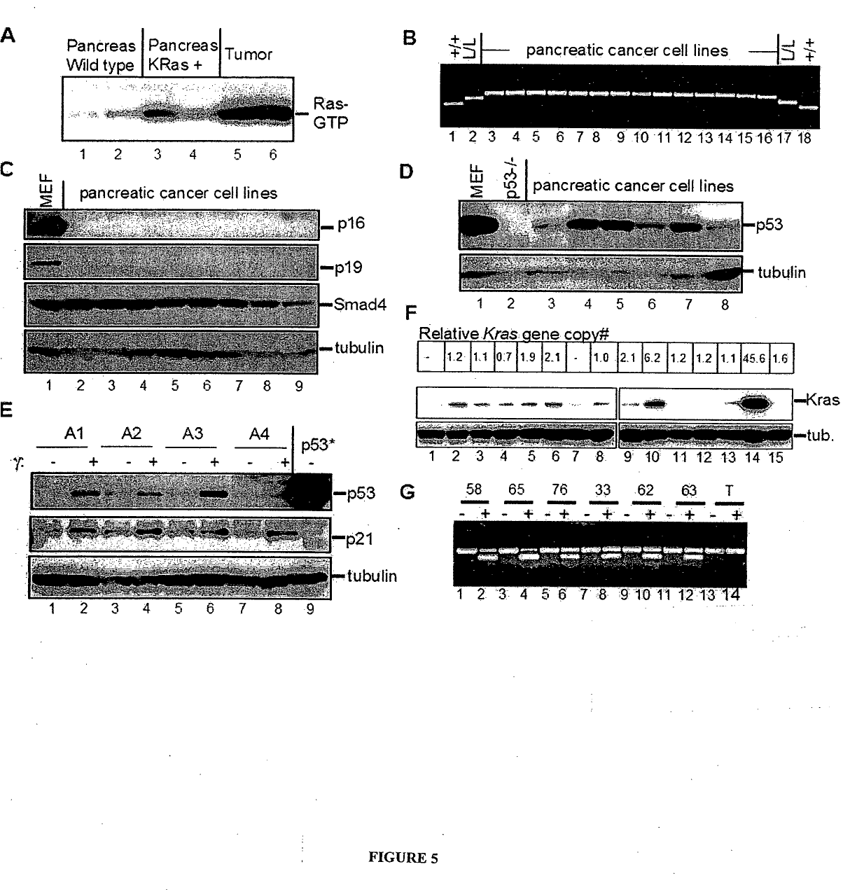

A molecular analysis of the tumors was conducted to determine the status of pathways that are commonly altered in human pancreatic adenocarcinomas. Whole pancreas lysates from Pdx1-Cre; LSL-Kras^{G12D} mice showed a modest elevation in Ras-GTP compared to age-matched wildtype controls (FIG. 5a), consistent with expression of the mutant constitutively active Kras^{G12D} allele. Furthermore, levels of Ras-GTP were significantly elevated in Pdx1-Cre; LSL-Kras^{G12D}; Ink4a/Arf^{lox/lox} tumor lysates (FIG. 5a), indicating that the activated KRAS signaling pathway remains engaged and may even be further enhanced in advanced-stage tumors, although this increase could alternatively reflect the altered balance of cell types in the tumor versus normal pancreas.

Further molecular studies of tumor samples utilized early passage cell lines derived from the murine pancreatic adenocarcinomas (Materials and Methods) in order to avoid the presence of contaminating normal cells. As expected, the tumor cell lines showed homozygous deletion of the Ink4a/Arf locus as determined by PCR analysis (FIG. 5b) and did not express p16^{Ink4a} or p19^{Arf} (FIG. 5c). Next, the status of the Smad4 and p53 tumor suppressor genes was assessed; advanced human pancreatic adenocarcinomas often sustain homozygous deletion or truncating mutations of Smad4 resulting in loss of expression as well as p53 missense mutations resulting in stabilization of the mutant protein. In all cases examined (n=15), western blots showed robust expression of full length SMAD4 protein (FIG. 5c) and sequence analysis of the RT-PCR-generated open reading frame revealed wildtype Smad4 sequences (see Materials and Methods). In addition, Western blot analysis revealed modest levels of p53 (FIG. 5d) and showed induction of increased p53 and p21^{C1P1} levels in response to ionizing radiation (FIG. 5e), consistent with wild-type p53 function. Accordingly, sequence analysis of the RT-PCR-generated p53 open reading frame confirmed that all specimens had a wild-type p53 status. Gene copy number alterations at the KRAS locus are thought to contribute to the progression of some tumors harboring activating KRAS mutations. Specifically, KRAS gene amplification occurs in certain human malignancies including human pancreatic adenocarcinomas and in several tumor types in the mouse (Yamada, H., et al. (1986) Jpn J Cancer Res 77: 370-5; Mahlamaki, E. H., et al. (1997) Genes Chromosomes Cancer 20: 383-91; Liu, M. L., et al. (1998) Oncogene 17: 2403-11; Schleger, C., et al. (2000) J Pathol 191: 27-32; Heidenblad, M., et al. (2002) Genes Chromosomes Cancer 34: 211-23; O'Hagan, R. C., et al. (2002) Cancer Cell 2: 149-55). Additionally, loss of the wildtype RAS allele has also been shown to promote cellular transformation by activated RAS (Bremner, R. and A. Balmain (1990) Cell 61: 407-17; Finney, R. E. and J. M. Bishop (1993) Science 260: 1524-7; Zhang, Z., Y. et al. (2001) Nat Genet. 29: 25-33). Quantitative real-time PCR (QPCR) was performed on genomic DNA derived from 15 pancreatic cancer cell lines to assess the relative Kras gene copy numbers (see Materials and Methods). These analyses revealed high-level amplifications in two specimens (FIG. 5f upper panel, lanes 10 and 14, 6-fold and 45-fold respectively); these changes were also evident in the corresponding primary tumors (3-fold and 5.5-fold respectively). In addition, approximately 2-fold gains were detected in 3 other specimens (FIG. 5f upper panel, lanes 5, 6 and 9) and in the corresponding primary tumors. The reduced relative magnitude of the amplification in some primary tumor specimens may have been due to the presence contaminating stromal tissue. Immunoblot analysis revealed marked Kras expression increases in the lysates from cell lines that had high level gene amplifications and more modest increases in the lysates from lines with lower level gains (FIG. 5f, lower panels). Next, RT-PCR/RFLP analysis was employed to assess whether the mutant or wild-type allele is amplified in these tumors. These analyses revealed an increased intensity of the band corresponding to the mutant Kras^{G12D} transcript in the samples with Kras amplification (FIG. 5g, lanes 2 and 4), indicating that specifically the mutant allele is amplified. Finally, these data show that the expression of the wild-type Kras allele is also retained in all tumors, a result corroborated by PCR analysis of genomic DNA showing that both the wild-type and mutant Kras alleles were present (FIG. 5g and data not shown). Hence gene amplification of activated Kras, but not loss of the wild-type allele, appears to contribute to malignant progression in the pancreas.

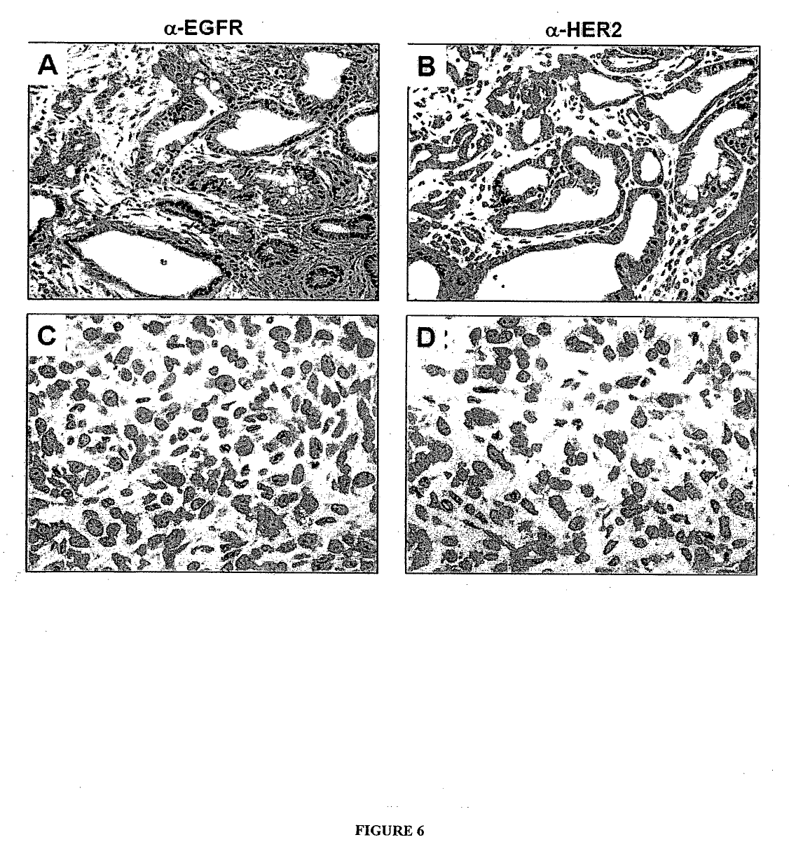

Finally, the status of accessory signaling pathways in these tumors was addressed. The activation and extinction of EGFR and HER2/NEU are characteristic of the evolving human disease. Specifically both EGFR and HER2/NEU are induced early in PanIN progression and remain elevated in ductal adenocarcinomas while HER2/NEU expression becomes extinguished in more advanced, undifferentiated tumors (Korc, M., et al. (1992) J Clin Invest 90: 1352-60; Day, J. D., et al. (1996) Hum Pathol 27: 119-24; Friess, H., et al. (1996) J Mol Med 74: 35-42). Likewise, in the mouse model, expression of both EGFR (FIG. 6a) and HER2/NEU (FIG. 6b) in the glandular component of the tumors but not in the undifferentiated, sarcomatoid regions (FIGS. 6c and 6d) was observed. In this model, the lack of Smad4 and p53 mutations, along with the activation of the growth signaling molecules, provides opportunity for the further analysis of these pathways in the biology of this disease.

Kras activation appears to be an initiating step in PanIN development in the mouse. The grossly normal pancreatic architecture despite universal Kras^{G12D} expression in Pdx1-Cre; LSL-Kras^{G12D} mice suggests that additional—possibly epigenetic—events are required to allow the emergence of PanINs. Equivalently, KRAS mutations have been observed in histologically normal human pancreas specimens (Luttges, J., et al. (1999) Cancer 85: 1703-10). In the presence of an intact Ink4a/Arf locus, the murine PanINs do not progress beyond the PanIN-2 stage by age 30 weeks despite their high multiplicity, which indicates that an efficient and potent p16^{INK4A} and/or p19^{ARF}-mediated checkpoint mechanism restrains malignant progression in initiated lesions. The Ink4a/Arf locus is critical in regulating both the progression of PanINs and the development of invasive pancreatic adenocarcinoma.

The onset of pancreatic adenocarcinomas by 6 weeks, the preceding rapid progression of PanINs to advanced stages and the absence of neoplastic change in other compartments provide evidence for a PanIN-to adenocarcinoma sequence in Pdx1-Cre; LSL-Kras^{G12D}; Ink/Arf^{lox/lox} mice. Likewise, it cannot be determined whether PanINs arise from differentiated ductal cells, resident multipotent progenitor cells or via transdifferentiation of acinar or other cell types. In fact, previous data have shown that Ink4a/Arf loss permits de-differentiation of mature astrocytes to glioblastoma in vivo (Bachoo, R. M., et al. (2002) Cancer Cell 1: 269-77), and therefore p16^{INK4A} and/or p19^{ARF} could be playing a similar role in blocking progression in this model. It is of interest to note that the expression of Cre early in pancreas development through the Pdx1 promoter enables efficient activation of Kras in all pancreatic compartments, yet only ductal tumors develop. This unique association of Kras activation and pancreatic ductal tumorigenesis appears applicable to human cancer of the pancreas since KRAS mutations are detected in ductal adenocarcinomas and in another type of pancreatic ductal malignancy, Intraductal Papillary Mucinous Neoplasms (Z'Graggen, K., et al. (1997) Ann Surg 226:491-8; discussion 498-500), but not in acinar cell tumors (Terhune, P. G., et al. (1994) Mol Carcinog 10: 110-4). The unbiased Pdx1-Cre system, therefore, indicates that, at physiological levels, either activated Kras drives progenitor cells or differentiated cells of various lineages toward a ductal phenotype, or that this oncoprotein is singularly able to exert its transforming effects in the duct compartment. Formal resolution of these questions will likely require rigorously defined cell culture-based systems as well as crosses to Cre recombinase strains specifically targeted to more differentiated pancreatic lineages.

The absence of PanIN lesions in Pdx1-Cre; Ink4a/Arf^{lox/lox} mice suggests that p16^{INK4A} and p19^{ARF} do not regulate the onset of these earliest neoplastic stages. Rather, the rapid progression of PanIN lesions and development of adenocarcinomas in the Pdx1-Cre; LSL-Kras^{G12D}; Ink4a/Arf^{lox/lox} mouse indicates that the Ink4a/Arf locus is required to restrain the malignant transformation of these initiated lesions. Such a role in progression rather than initiation fits well with the documented appearance of INK4A/ARF loss at the PanIN-2 stage in humans, and the comparable age-of-onset of pancreatic adenocarcinoma observed in a subset of families with germline INK4A mutations and that observed for sporadic tumors (Moskaluk, C. A., et al. (1997) Cancer Res 57:2140-3; Wilentz, R. E., et al. (1998) Cancer Res 58: 4740-4; Lynch, H. T., et al. (2002) Cancer 94: 84-96). The mechanistic basis for the in vivo cooperative interactions of activated KRAS and INK4A/ARF deficiency remains an important issue. Until recently, a prevailing model has proposed the existence of a feedback loop whereby activated RAS mediates induction of MAPK kinase leading directly to induction of Ink4a and Arf and subsequent growth arrest (Serrano, M., et al. (1996) Cell 85: 27-37). However, recent studies have shown that such a direct loop is not operative in response to endogenous—as opposed to overexpressed—levels of activated RAS (Guerra, C., et al. (2003) Cancer Cell 4: 111-20). It appears that physiological expression of this locus is tightly controlled by both positive and negative regulators, possibly modulated by such factors as integrin-extracellular matrix interactions (Plath, T., et al. (2000) J Cell Biol 150: 1467-78; Natarajan, E., et al. (2003) Am J Pathol 163: 477-91) and mitogenic stimuli (Alani, R. M., et al. (2001) Proc Natl Acad Sci USA 98:7812-6; Ohtani, N., et al. (2001) Nature 409: 1067-70). Without intending to be bound by theory, these finding indicate that alterations in the balance of these signals occur in PanIN lesions, but not in the normal pancreas (with or without Ras activation), resulting in activation of the Ink4a/Arf locus.

Another important issue in human pancreatic adenocarcinoma is the relative pathogenic roles of INK4A versus ARF loss. In humans, INK4A mutation seems to determine disease predisposition as there are both sporadic and germline mutations that specifically target INK4A yet spare ARF (Rozenblum, E., M. et al. (1997) Cancer Res 57: 1731-4; Liu, L., et al. (1999) Nat Genet. 21: 128-32; Lal, G., et al. (2000) Genes Chromosomes Cancer 27: 358-61; Lynch, H. T., et al. (2002) Cancer 94: 84-96). On the other hand, the loss of ARF—via homozygous deletion of the locus—occurs in about 50% of the tumors (Rozenblum, E., M. et al. (1997) Cancer Res 57: 1731-4). Notably, a significant proportion of these ARF-deficient tumors also harbor p53 mutations (Rozenblum, E., M. et al. (1997) Cancer Res 57: 1731-4), potentially reflecting an oncogenic role of ARF loss other than in p53 regulation and/or the specific role of p53 loss on the DNA damage response (see below). The specific tumor suppressor activities contributed by Ink4a and by Arf should be resolved by crosses of the Pdx1-Cre; LSL-Kras^{G12D} mice onto genetic backgrounds deficient for either gene of this locus. It is notable that the p53 mutations were absent in the mouse tumor model. This may reflect the need to inactivate the p19^{ARF}-independent DNA damage sensing function of p53 in human cancer but not in the mouse. Thus, in the Pdx1-Cre; LSL-Kras^{G12D}; Ink4a/Arf^{lox/lox} mice, Arf deletion—which neutralizes p53 induction by other stresses such as aberrant cell cycle entry and activated oncogene expression—may effectively substitute for p53 loss in tumor progression. One basis for this divergence may be the cross species differences in telomere dynamics (Maser, R. S, and R. A. DePinho (2002) Science 297: 565-9), the prominent role of p53 (and not ARF) in the telomere checkpoint response of evolving tumors (Chin, L., et al. (1999) Cell 97: 527-38), and evidence of telomere dysfunction in the progression of human pancreatic adenocarcinoma (van Heek, N. T., et al. (2002) Am J Pathol 161: 1541-7).

Lastly, the data indicate that many of the classical features of malignancy in general and of pancreatic cancer in specific can be recapitulated by Ink4a/Arf loss in the setting of KRAS activation. Whereas KRAS activation or Ink4a/Arf loss alone does not cause local invasion and advanced local growth, the combination does.

Analyses of the mouse model directed by oncogenic Kras and deletion of the Ink4/Arf tumor suppress genes, described above, have shown that Kras activation constitutes an initiating lesion for pancreatic adenocarcinoma but is insufficient for malignant progression. While the Ink4a/Arf locus does not contribute to initiation, the integrity of this locus is critical in restraining the malignant progression of Kras induced lesions. These studies have been extended to investigate the genetic interactions in pancreatic adenocarcinoma pathogenesis by analyzing the p53, Ink4a (with intact Arf), Smad4, and Lkb1 tumor suppressors against the backdrop of Kras activation, e.g., Pdx1-Cre; LSL-Kras^{G12D}; Ink4a^{lox/lox}; Pdx1-Cre; LSL-Kras^{G12D}; p53^{lox/lox}; Pdx1-Cre; LSL-Kras^{G12D}; SMAD4^{lox/lox}; Pdx1-Cre; LSL-Kras^{G12D}; Lkb1^{lox/lox}, mice have been generated. The data indicate that 1) p53 deletion can effectively replace the need for Ink4a/Arf loss in the promotion of pancreatic adenocarcinoma, 2) Ink4a deletion is insufficient to promote Kras-driven tumorigenesis, 3) Smad4 deletion promotes enhanced invasive and metastatic tumor growth and 4) Lkb1 deletion effectively cooperates with Kras activation. These genetic model systems provide a platform for dissecting the oncogenic circuitry engaged by these mutant alleles and for identifying surrogate biomarkers of their activity. Specifically the availability of large numbers of genetically identical animals undergoing synchronous tumorigenesis allows isolation of specimens at progressive stages of the tumorigenic process, an opportunity that is unavailable in association with the human disease.

### Example 2

### Biomarker Discovery Utilizing Animal Models of Pancreatic Adenocarcinoma

Having established the histologic and clinically validity of the animal models of the invention, the applicability of these models for the identification of pancreatic cancer specific serum biomarkers was addressed. In addition, to the comprehensively identification of stage-specific biomarkers for pancreatic adenocarcinoma, biomarkers that are specific for particular genetic lesions, including loss of function of Smad4, p53, Ink4a, Arf, and Lkb1 were developed, e.g., Pdx1-Cre; LSL-Kras^{G12D}; Ink4a^{lox/lox}; Pdx1-Cre; LSL-Kras^{G12D}; p53^{lox/lox}; Pdx1-Cre; LSL-Kras^{G12D}; SMAD4^{lox/lox}. These biomarkers will serve to allow the development of highly sensitive, rapid and cost-effective screening methods for detection of early stage disease in clinically asymptomatic subjects as well as for diagnosis of various stages of pancreatic cancer. A non-invasive test involving serum analysis allows the rapid evaluation of subjects who may be at risk in the context of routine clinical examination.

An important component of biomarker discovery is the need to develop sensitive, non-invasive imaging techniques to monitor the development and growth of pancreatic tumors in the model system of the present invention in order to permit serial serum analysis and correlation with cancer progression. To this end, optimized protocols for magnetic resonance imaging (MRI) detection of evolving murine pancreatic cancers have been developed. Furthermore, there was a need to evaluate existing proteomics approaches for their suitability in the biomarker discovery efforts. To this end, serum from mice with a moderate tumor burden and from control littermate mice was isolated. The serum specimens were analyzed by the Eprogen ProteoSep platform, a novel protein discovery chemistry and software platform that employs automated analytical techniques for high-resolution protein analysis. ProteoSep is gel-free, all-liquid phase protein mapping technology that uses liquid chromatographic techniques to produce high-resolution 2D maps of complex protein systems. First dimension separation of proteins based on their pI and second dimension hydrophobicity separations are achieved using a unique high-performance chromatofocusing (CF) and high-resolution reverse phase NPS protein separation columns, respectively. A 2D protein map is then produced using Eprogen's ProteoSep Software Suite, displaying the complete protein profile for the sample. The analysis demonstrated map profiles that showed clear clustering of serum specimens from tumor bearing mice and control mice, respectively and that revealed the existence of numerous specific cancer-associated protein peaks. These data serve as a definitive proof-of-principle for the utility of the murine tumor model in conjunction with the Eprogen ProteoSep system as a biomarker discovery platform. Importantly, the Eprogen system resolves the serum specimens into 9×96 liquid fractions that can readily be subject to mass spectrometry analysis for identification of specific peptides once 2D plots have identified potential biomarkers. Those skilled in the art would recognize that other proteomics platforms could be used to achieve the same results such as those employing other chromatography approaches (for example the Protein Forest ProteomeChip™) and specific protein chips (such as the Zyomyx cytokine chips).

The specific approach for biomarker discovery and validation is as follows: Tumor-prone and control mice were subjected to serial (weekly) serum isolation and monitoring by MRI; the serum specimens were profiled by the Eprogen system or another proteomics platform followed by mass spectrometry in order to allow the identification of stage-specific markers. Antibodies are raised to these markers. Epitopes for antibody generation are chosen to also recognize the orthologous protein in humans. These antibodies are evaluated for their applicability as human diagnostic markers using ELISA-based assays to test serum from human pancreatic cancer subjects and from subjects in prospective studies whose clinical follow-up reveals the development of pancreatic adenocarcinoma.

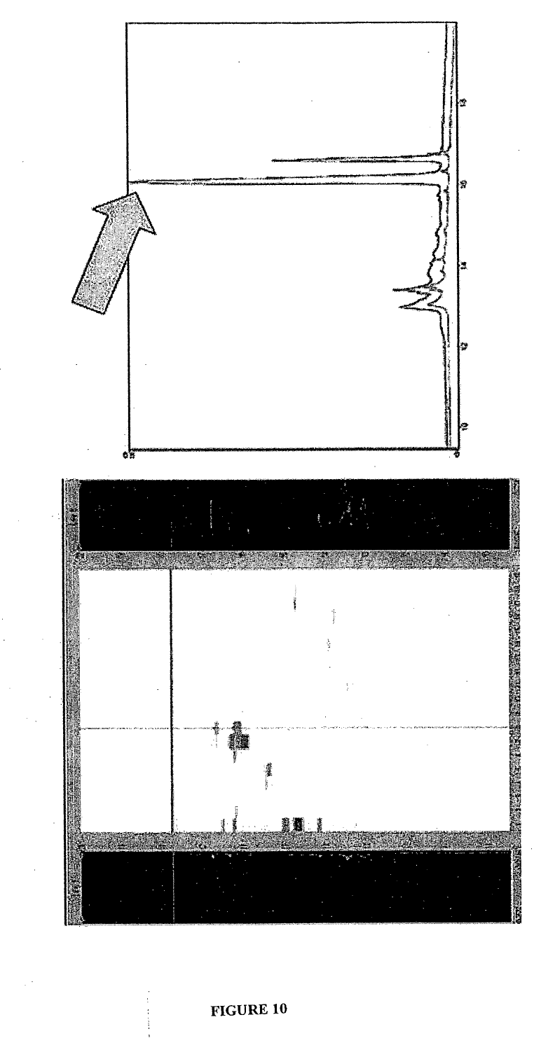

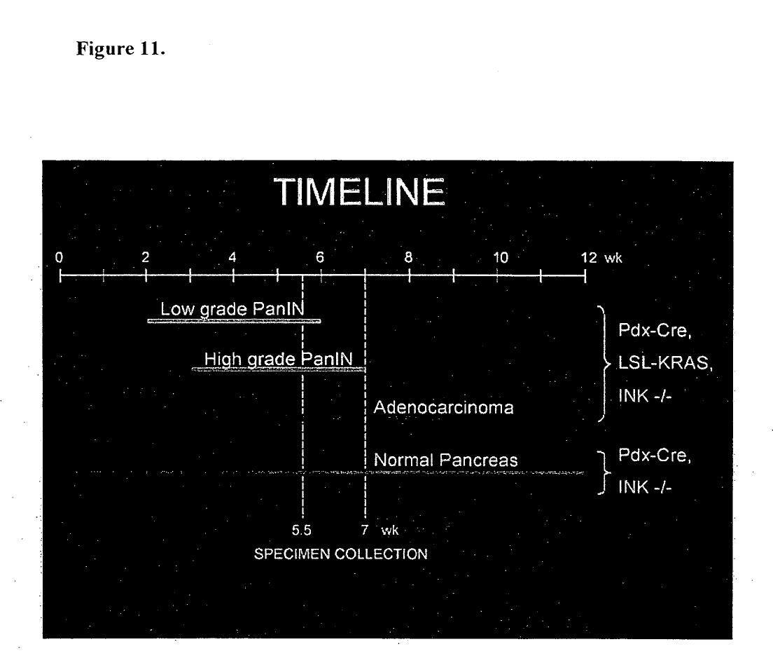

These efforts have identified specific biomarkers of moderately advanced disease (established malignant tumors but no invasion of adjacent organs or metastasis) in Pdx1-Cre; LSL-Kras^{G12D}; Ink4a/Arf^{lox/lox} mice (FIG. 10). FIG. 11 depicts the timeline of tumor progression in the Pdx1-Cre; LSL-Kras^{G12D}; Ink4a/Arf^{lox/lox} model and indicates the points at which specimens were collected for this analysis.

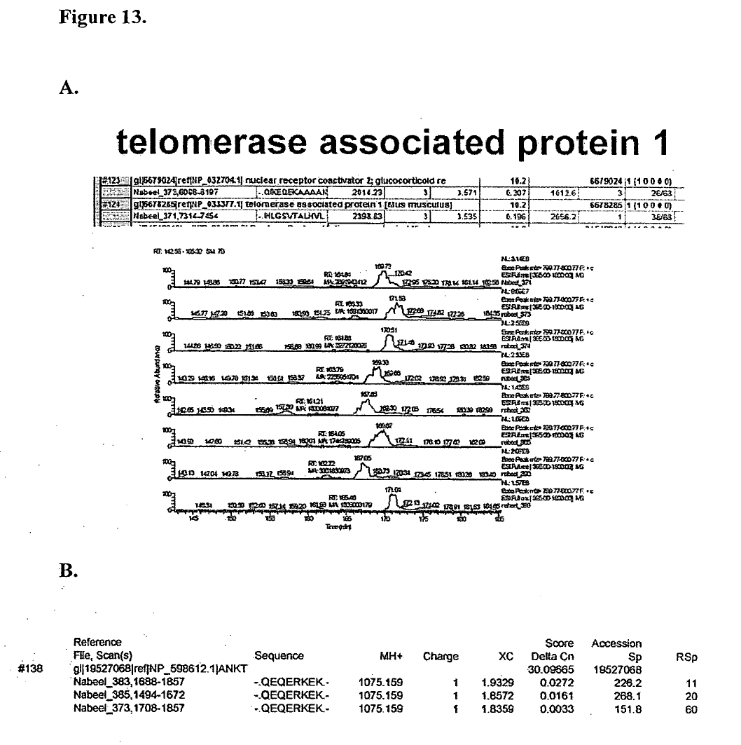

Subsequent to these analyses, LC-MS was employed, either directly or on tryptic digests of plasma specimens or following prefractionation (Eprogen or Lectin binding). Using informatics methods, the clustering of disease profiles versus control profiles have been performed and specific peptides with high correlation to the disease state have been identified, such as those shown in FIGS. 13A and B, and include but are not limited to HLGSVTALHVL (SEQ ID NO:23) and QEQERKEK (SEQ ID NO.:24) (FIG. 13B). FIG. 13A shows cancer-specific expression of telomerase-associated protein and of the protein, ANKT, that were both identified in unfractionated plasma. ANKT, also known as nucleolar spindle-associated protein (NuSAP), is crucial in spindle microtubule organization and is selectively expressed in proliferating cells.

### Example 3

### Biomarker Discovery Utilizing Animal Models of Pancreatic Adenocarcinoma

Human solid tumors often undergo extensive chromosomal rearrangements and display marked cytogenetic abnormalities, including amplifications and deletions, leading to gain or loss of normal chromosomal material and translocations, or abnormal fusions between two different chromosomes, leading to the production of novel chimeric proteins. This process of “genomic instability” occurs through disruption of the molecular mechanisms governing the integrity of a cell's chromosomal material and the resultant cytogenetic aberrations are thought to contribute to the pathogenesis of malignant neoplasms.

Genomic instability is a hallmark feature of pancreatic adenocarcinoma and numerous cytogenetic studies of human tumor specimens have indicated a profound number of recurrent amplifications and deletions within the pancreatic cancer genome. While these genetic lesions point to the existence of many potentially novel oncogenes and tumor suppressor genes in this disease, the level of extreme genomic complexity in human pancreatic cancers also complicates identification of those lesions that are most critical to the pathogenesis of this neoplasm. Studies of the cytogenetic abnormalities occurring in faithful mouse models of pancreatic cancer may serve as valuable filters to help identify those events occurring in human tumors with primary diagnostic and pathogenic importance. Furthermore, the ability to genetically manipulate alleles in mice allows one to rigorously address the mechanistic role of oncogenic lesions in generating genomic instability during tumorigenesis.

In order to identify the MCRs of the present invention, novel cDNA or oligomer-based platform and bioinformatics tools were utilized. These methods allowed for the high-resolution characterization of copy-number alterations in the pancreatic cancer genome, e.g., the pancreatic adenocarcinoma genome.

To arrive at the MCRs, array comparative genomic hybridization (array-CGH) was utilized to define copy number aberrations (CNAs) (gains and losses of chromosomal regions) in pancreatic adenocarcinoma cell lines and tumor specimens (see, e.g., Table 2).

Segmentation analysis of the raw profiles to filter noise from the dataset (as described by Olshen and Venkatraman, Olshen, A. B., and Venkatraman, E. S. (2002) ASA Proceedings of the Joint Statistical Meetings 2530-2535; Ginzinger, D. G. (2002) Exp Hematol 30, 503-12; Golub, T. R., et al. (1999) Science 286, 531-7; Hyman, E., et al. (2002) Cancer Res 62, 6240-5; Lucito, R., et al. (2003) Genome Res 13, 2291-305) was performed and used to identify statistically significant changepoints in the data.

Identification of loci within the MCRs was based on an automated computer algorithm that utilized several basic criteria as follows: 1) segments above or below certain percentiles were identified as altered; 2) if two or more altered segments were adjacent in a single profile separated by less than 500 KB, the entire region spanned by the segments was considered to be an altered span; 3) highly altered segments or spans that were shorter than 20 MB were retained as “informative spans” for defining discrete locus boundaries. Longer regions were not discarded, but were not included in defining locus boundaries; 4) informative spans were compared across samples to identify overlapping groups of positive-value or negative-value segments; each group defines a locus; and 5) MCRs were defined as contiguous spans having at least 75% of the peak recurrence as calculated by counting the occurrence of highly altered segments. If two MCRs were separated by a gap of only one probe position, they were joined. If there were more than 3 MCRs in a locus, the whole region was reported as a single complex MCR.

A locus-identification algorithm was used that defines informative CNAs on the basis of size and achievement of a high significance threshold for the amplitude of change. Overlapping CNAs from multiple profiles were then merged in an automated fashion to define a discrete “locus” of regional copy number change, the bounds of which represent the combined physical extend to these overlapping CNAs. Each locus was characterized by a peak profile, the width and amplitude of which reflect the contour of the most prominent amplification or deletion for that locus. Furthermore, within each locus, one or more MCRs were identified across multiple tumor samples, with each MCR potentially harboring a distinct cancer-relevant gene targeted for copy number alteration across the sample set.

The locus-identification algorithm defined discrete MCRs within the dataset which were annotated in terms of recurrence, amplitude of change and representation in both cell lines and primary tumors. These discrete MCRs were prioritized based on four criteria that emphasize recurrent high-threshold changes in both primary tumors and cell lines. Implementation of this prioritization scheme yielded MCRs of the present invention that satisfied at least three of the four criteria (see Table 2).

The confidence-level ascribed to these prioritized loci is further validated by real-time quantitative PCR (QPCR), which demonstrated 100% concordance with X selected MCRs defined by array-CGH.

In Table 2, the loci and MCRs are indicated as having either “gain and amplification” or “loss and deletion,” indicating that each locus and MCR has either (1) increased copy number and/or expression or (2) decreased copy number and/or expression, or deletion, in cancer. Furthermore, genes known to play important roles in the pathogenesis of pancreatic cancer (such as, p16^{INK4a}, Kras2, SMAD4, LKB1, and telomerase) are present within the loci and are also set forth in Table 2.

Complementary expression profile analysis of a significant fraction of the genes residing within the MCRs of the present invention provides a subset of biomarkers with statistically significant association between gene dosage and mRNA expression. Additional biomarkers within the MCRs that have not yet been annotated may also be used as biomarkers for cancer as described herein, and are included in the invention.

The novel methods for identifying chromosomal regions of altered copy number, as described herein, may be applied to various data sets for various diseases, including, but not limited to, cancer. Other methods may be used to determine copy number aberrations are known in the art, including, but not limited to oligonucleotide-based microarrays (Brennan, et al. (2004) In Press; Lucito, et al. (2003) Genome Res. 13:2291-2305; Bignell et al. (2004) Genome Res. 14:287-295; Zhao, et al (2004) Cancer Research, 64(9):3060-71), and other methods as described herein including, for example, hybridization methods (such as, for example, FISH and FISH plus spectral karyotype (SKY)).

### A. Materials and Methods

### Cell Lines and Primary Tumors.

All the primary tumors were acquired from Pdx-Cre; LSL-Kras^{G12D} mice harboring either the Ink4a/Arf or p53 conditional tumor suppressor alleles. Mice were allowed to generate pancreatic adenocarcinomas and low passage cell lines were derived from these tumors. Mice harboring other conditional tumor suppressor alleles, e.g., Pdx1-Cre; LSL-Kras^{G12D}; Ink4a^{lox/lox}; Pdx1-Cre; LSL-Kras^{G12D}; p53^{lox/lox}; Pdx1-Cre; LSL-Kras^{G12D}; SMAD4^{lox/lox}; Pdx1-Cre; LSL-Kras^{G12D}; Lkb1^{lox/lox}, were also allowed to develop tumors which are used for the analyses as described below

Array-CGH Profiling on cDNA Microarrays.

Genomic DNAs from cell lines and primary tumors were extracted according to the manufacturer instruction (Gentra System Lie, Minneapolis, Minn.). Genomic DNA was fragmented and random-prime labeled according to published protocols (Pollack, J. R., et al. (1999a) Nat Genet. 23, 41-46.) with modifications (For details, see, Aguirre, A. J., et al. (2004) Proc Natl Acad Sci USA 101, 9067-72). Labeled DNAs were hybridized to either human cDNA microarrays or oligo microarrays. The cDNA microarrays contain 14,160 cDNA clones (Agilent Technologies, Human 1 clone set) with 13,281 mappable clones, for which approximately 11,211 unique map positions were defined (NCBI, Build 34).). The median interval between mapped elements is 72.7 kilobase; 94.1% of intervals are less than 1 Mb, and 98.9% are less than 3 Mb. The oligo array contains 22K oligonucleotides (Agilent Technologies). All probes were subjected to BLAST alignment with the latest draft of human/mouse genome sequence (NCBI, Build 34). Based on the BLAST results, un-mappable probes were eliminated, which were arbitrarily defined as alignment length of best hit less than 55 bp; and un-informative probes, which were identified by having a second best hit score of 95% or higher on the alignment length of the best hit. These criteria selected 16022 informative probes for the human array. The median interval between mapped elements is 54.7 kilobase, 96.7% of intervals are less than 1 Mb, and 99.5% are less than 3 Mb.

Fluorescence ratios of scanned images of the arrays were calculated and the raw array-CGH profiles were processed to identify statistically significant transitions in copy number using a segmentation algorithm which employs permutation to determine the significance of change points in the raw data (Olshen, A. B., et al. (2004) Biostatistics 5(4):557-72). Each segment was assigned a Log₂ ratio that is the median of the contained probes. The data was centered by the tallest mode in the distribution of the segmented values. After mode-centering, gains and losses were defined as Log₂ ratios of greater than or equal to +0.11 or −0.11 (+/−4 standard deviations of the middle 50% quantile of data), and amplification and deletion as a ratio greater than 0.4 or less than −0.4, respectively. The comparison between squamous and adenocarcinomas was performed as follows: a custom-made algorithm was designed to identify all the probes on the segmented data that were above a Log₂ ratio of 0.5 in at least 25% of the samples in one tumor subtype and absent in the other dataset. For the deletions, the algorithm searched for all the probes on the segmented data that were below a Log₂ ratio of 0.5 in at least 25% of the samples in one tumor subtype and absent in the other dataset.

### Automated Locus Definition.

Loci are defined by an automated algorithm applied to the segmented data based on the following rules:

1. Segments above 0.4 or below −0.4 were identified as altered.

2. If two or more altered segments are adjacent in a single profile or separated by less than 500 KB, the entire region spanned by the segments was considered to be an altered span.

3. Highly altered segments or spans that are shorter than 20 MB were retained as “informative spans” for defining discrete locus boundaries. Longer regions were not discarded, but were not included in defining locus boundaries.

4. Informative spans were compared across samples to identify overlapping groups of positive-value or negative-value segments; each group defines a locus.

5. Overlap groups were divided into separate groups wherever the recurrence rate falls below 25% of the peak recurrence for the whole group. Overlap groups were divided into separate groups wherever the recurrence rate falls below 25% of the peak recurrence for the whole group. Recurrence was calculated by counting the number of samples with alteration at high threshold (0.4, −0.4).

6. MCRs were defined as contiguous spans having at least 75% of the peak recurrence as calculated by counting the occurrence of highly altered segments. If two MCRs were separated by a gap of only one probe position, they are joined. If there are more than 3 MCRs in a locus, the whole region is reported as a single complex MCR.

### MCR Characterization.

For each MCR, the peak segment value was identified. Recurrence of gain or loss was calculated across all samples based on the lower thresholds previously defined (˜+/−0.11). As an additional measure of recurrence independent of thresholds for segment value or length, Median Aberration (MA) was calculated for each probe position by taking the median of all segment values above zero for amplified regions, below zero for deleted regions. This pair of values was compared to the distribution of values obtained after permuting the probe labels independently in each sample profile. Where the magnitude of the MA exceeded 95% of the permuted averages, the region was marked as significantly gained or lost, and this is used in the voting system for prioritization. The number of known genes is counted based on the July 2003 human assembly at NCBI (build 34).

### Quantitative PCR (QPCR) Verification.

PCR primers are designed to amplify products of 100-150 bp within target and control sequences. Primers for control sequences in each cell line are designed within a region of euploid copy number as shown by array-CGH analysis. Quantitative PCR is performed, by monitoring in real-time, the increase in fluorescence of SYBR Green dye (Qiagen, Valencia, Calif.) with an ABI 7700 sequence detection system (Perkin Elmer Life Sciences, Boston, Mass.). Relative gene copy number is calculated by the standard curve method (Ginzinger, D. G. (2002) Exp Hematol 30, 503-512). Estimates of gene dosage are made relative to the most common copy number within a sample. For PCR validation, abundant Line elements are used as a reference against which to compare copy number alterations. Based on previous experience with other datasets (Aguirre, A. J., et al. (2004) Proc Natl Acad Sci USA 101, 9067-9072; Brennan, C., et al. (2004) Cancer Res 64, 4744-4748), a threshold of 2 (as relative gene dosage) is used as a cutoff of alteration. For validation of expression, RNA-specific PCR primers are designed to amplify products of 100-150 bp across exons.

### Expression Profiling on Affymetrix GeneChip.

Biotinylated target cRNA is generated from total sample RNA and hybridized to human oligonucleotide probe arrays (U133Plus 2.0, Affymetrix, Santa Clara, Calif.) according to standard protocols (Golub, T. R., et al. (1999) Science 286, 531-537). Expression values are normalized in dChip (Li, C., and Wong, W. H. (2001) Proc Natl Acad Sci USA 98, 31-36) and then for each gene are standardized by Log₂ ratio to the median of the cohort.

Integrated Copy Number and Expression Analysis.

Array-CGH data is interpolated such that each expression value can be mapped to its corresponding copy number value. For each gene position, the samples are grouped based on whether array-CGH showed altered copy number or not based on interpolated CGH value. The effect of gene dosage on expression is measured by calculating a gene weight defined as the difference of the means of the expression value in the altered and unaltered sample groups divided by the sum of the standard deviations of the expression values in altered and unaltered sample groups (Aguirre, A. J., et al. (2004) Proc Natl Acad Sci USA 101, 9067-9072; Hyman, E., et al. (2002) Cancer Res 62, 6240-6245). The significance of the weight for each gene is estimated by permuting the sample labels 10,000 times and applying an alpha threshold of 0.05.

### Fluorescence In Situ Hybridization (FISH).

Metaphase spread slides are prepared following standard protocols (Protopopov, A. I., et al. (1996) Chromosome Res 4, 443-447). Frozen tissue sections (4 μm) are pre-treated according to manufacturer's protocol (Frozen Tissue Prep for FISH, Vysis, Downers Grove, Ill.). The probes for FISH analysis are labeled using nick translation, according to the manufacturer's instructions (Roche Molecular Biochemicals, Indianapolis, Ind.) with either biotin-14-dATP or digoxigenin-11-dUTP. Biotinylated probes are detected using Cy3-conjugated avidin (Accurate Chemical, Westbury, N.Y.). For digoxigenin-labeled probes, antidigoxigenin-FITC Fab fragments (Enzo Life Sciences, Farmingdale, N.Y.) are used. Slides are counterstained with 5 μg/ml 4′,6-diamidino-2-phenylindole (Merck, Wilmington, Del.) and mounted in Vectashield antifade medium (Vector Laboratories, Burlingame, Calif.). FISH signals acquisition and spectral analysis are performed using filter sets and software developed by Applied Spectral Imaging (Carlsbad, Calif.).

### Identification of Known and Novel CNAs in the Pancreatic Adenocarcinoma Genome.

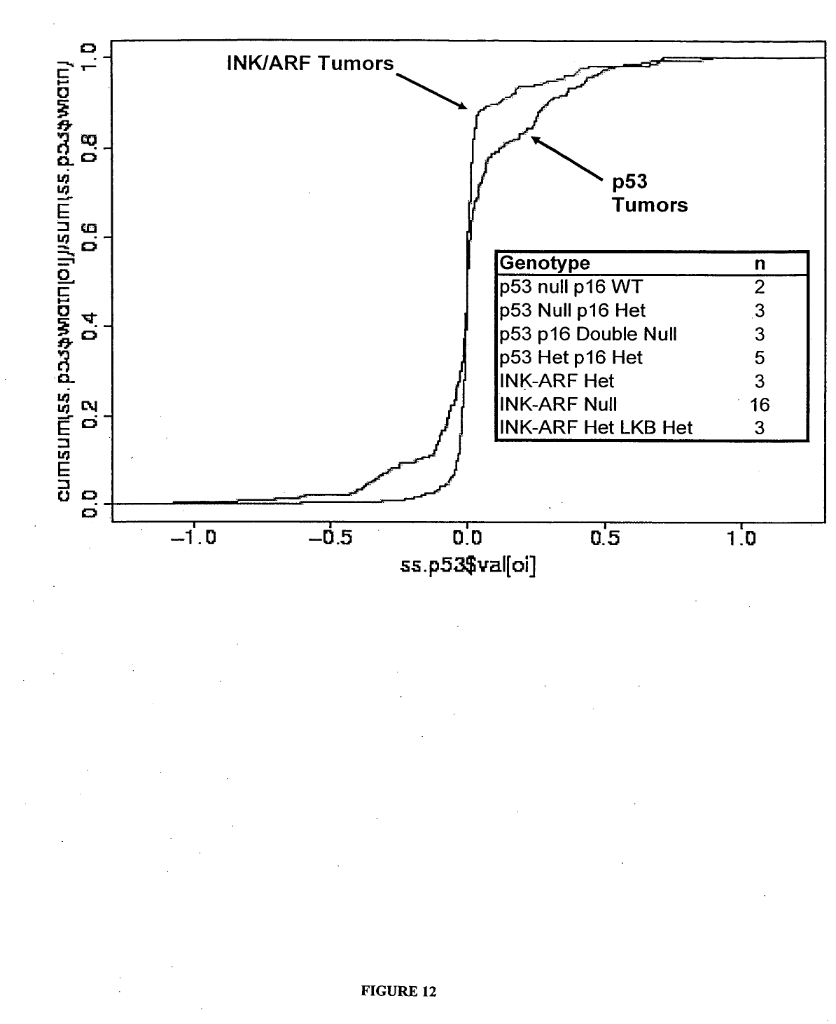

To identify genomic aberrations occurring in the Kras^{G12D}-driven mouse model of pancreatic adenocarcinoma, an array-comparative genomic hybridization was utilized to comprehensively assess alterations in chromosomal copy number. Models of pancreatic adenocarcinoma utilizing the Ink4a/Arf or p53 tumor suppressor alleles were generated as described herein and 35 tumors have been collectively analyzed by array-CGH. These profiles comprise an extensive database of valuable information regarding the biology and genetics of pancreatic tumors. First, the differential impact of inactivation of the Ink4a/Arf or p53 tumor suppressor genes on genomic instability became apparent when the genomic complexity of Ink4a/Arf-mutant tumors is compared to that of p53-mutant tumors (FIG. 12). Tumors containing mutations in the p53 tumor suppressor have increased genomic instability and more frequent cytogenetic aberrations (FIG. 12). These analyses are currently extending to assess other known pancreatic cancer genes including SMAD4, LKB1 and telomerase.

Utilizing custom bioinformatic approaches, a comprehensive list of copy number alterations occurring in the genomes of these mouse tumors was generated (Table 2). The columns in Table 2 have the following labels: Chr, “Chromosome”; MCR.St., the base pair start position for a given alteration; MCR.End., the base pair end position for the same alteration; MCR.Width (Mb), the size of the amplification or deletion in Mb; Peak.Val, the peak ACGH value for the CNA; Rec.All, Recurrence of the amplification or deletion across the dataset; MCR.genes, the number of genes within the amplification or deletion; Candidates, known candidate oncogenes or tumor suppressor genes within the amplification or deletion.

The list of MCRs in Table 2 represents an enumeration of all regions of gain or loss in these mouse tumors and outlines those chromosomal locations encompassing genes that may have pathogenic and/or diagnostic and therapeutic importance for pancreatic cancer. For example, genes located within regions of gain/amplification in these tumor genomes are overexpressed at the RNA and protein levels and thus are valid candidates for diagnostic biomarkers or novel therapeutic targets.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed:

1. A non-human animal model of pancreatic adenocarcinoma comprising an activating mutation of KRAS and one or more tumor suppressor genes or loci that is misexpressed.

2. The non-human animal model of claim 1, wherein said one or more tumor suppressor genes are conditionally misexpressed.

3. The non-human animal model of claim 1, wherein the activating mutation of KRAS is a Kras^{G12D} knock-in allele (LSL-Kras).

4. The non-human animal model of claim 1, wherein the activating mutation of KRAS is a Kras^{G12D} knock-in allele (LSL-Kras), and wherein the tumor suppressor gene is INK4a/Arf.

5. The non-human animal model of claim 1, wherein said animal comprises Pdx1-Cre; LSL-Kras^{G12D}; Ink4a/Arf^{lox/lox}.

6. The non-human animal model of claim 1, wherein said misexpression results in decreased expression of one or more tumor suppressor genes or loci.

7. The non-human animal model of claim 1, wherein the tumor suppressor gene is Ink4a/ARF.

8. The non-human animal model of claim 1, wherein the tumor suppressor gene is selected from the group consisting of Ink4a/ARF, Ink4a, Arf, p53, Smad4/Dpc, Lkb1, Brca2, and Mlh1.

9. The non-human animal model of claim 1, wherein the tumor suppressor genes are Ink4a/ARF and p53.

10. The non-human animal model of claim 1, wherein said one or more tumor suppressor genes or loci are disrupted by removal of DNA encoding all or part of the tumor suppressor protein.

11. The non-human animal model of claim 10, wherein said animal is homozygous for the one or more disrupted genes or loci.

12. The non-human animal model of claim 10, wherein said animal is heterozygous for the one or more disrupted genes or loci.

13. The non-human animal model of claim 1, wherein said animal is a transgenic animal with a transgenic disruption of said one or more tumor suppressor genes or loci.

14. The non-human animal model of claim 13, wherein the pancreatic and duodenal homeobox gene 1 (Pdx1)-Cre transgene is used to delete said one or more tumor suppressor genes or loci in the pancreas.

15. The non-human animal model of claim 1, wherein said animal is a rodent.

16. The non-human animal model of claim 15, wherein said rodent is a mouse.

17. A method for identifying for a biomarker associated with pancreatic adenocarcinoma comprising:
comparing the amount, structure, and/or activity of genes or proteins in a sample from an animal model of pancreatic adenocarcinoma, versus the presence, absence, or level of expression or activity of genes or proteins in a sample from a control wild-type animal, wherein the animal model comprises an activating mutation of KRAS and wherein one or more tumor suppressor genes or loci are misexpressed,
and wherein a difference in the amount, structure, and/or activity of a gene or protein indicates that the gene or protein is a biomarker associated with pancreatic adenocarcinoma.

18. The method of claim 17, wherein the identified biomarker is a diagnostic biomarker.

19. The method of claim 17, wherein the identified biomarker is a prognostic biomarker.

20. A method for identifying for a pharmacogenomic biomarker, wherein the pharmacogenomic biomarker is expressed in conjunction with a therapy regime comprising:
comparing the amount, structure, and/or activity of genes or proteins in a sample from an animal model of pancreatic adenocarcinoma, versus the presence, absence, or level of expression or activity of genes or proteins in a sample from a control wild-type animal, wherein the animal model comprises an activating mutation of KRAS and wherein one or more tumor suppressor genes or loci are misexpressed, wherein said animal model is administered a therapy regime;
and wherein a difference in the amount, structure, and/or activity of a gene or protein between the animal model sample and the control sample indicates that the gene or protein is a pharmacogenomic biomarker associated with pancreatic adenocarcinoma.

21. The method of claim 17 or 20, wherein said sample contains blood, urine, stool, bile, pancreatic cells or pancreatic tissue.

22. A biomarker identified by the method of claim 17.

23. The biomarker of claim 22, wherein said biomarker is a nucleic acid molecule.

24. The biomarker of claim 22, wherein said biomarker is a protein.

25. The method of claim 22, wherein said animal model displays metastatic pancreatic tumors.

26. The method of claim 22, wherein said animal model is asymptomatic for pancreatic adenocarcinoma.

27. A method for identifying a biomarker associated with pancreatic adenocarcinoma, said method comprising:
a) performing profiling of the genome of cancer cells, wherein said cells are from an animal model of pancreatic adenocarcinoma, wherein the animal model comprises an activating mutation of KRAS and wherein one or more tumor suppressor genes or loci are misexpressed;
b) performing segmentation analysis of profiles identified in step a);
c) identifying loci;
d) prioritizing said identified loci; and
e) interrogating genes in the identified loci,
to thereby identify a biomarker associated with pancreatic adenocarcinoma.

28. A method for identifying a locus associated with pancreatic adenocarcinoma, said method comprising the steps of:
a) performing profiling of the genome of cancer cells, wherein said cells are from an animal model of pancreatic adenocarcinoma, wherein the animal model comprises an activating mutation of KRAS and wherein one or more tumor suppressor genes or loci are misexpressed;
b) performing segmentation analysis of profiles identified in step a);
c) identifying loci; and
d) prioritizing said identified loci,
to thereby identify a locus associated with pancreatic adenocarcinoma.

29. The method of claim 27, wherein said interrogation of genes in the identified loci is based on gene expression data.

30. The method of claim 27, wherein said interrogation of genes in the identified loci is based on in vitro screening assays.

31. The method of claim 27 or 28, wherein said profiling is performed using comparative genomic hybridization (CGH).

32. The method of claim 27 or 28, wherein said cancer cells are derived from a pancreatic adenocarcinoma cell line or a pancreatic adenocarcinoma tumor.

33. A biomarker identified by the method of claim 28.

34. A method of identifying a gene or protein involved in stromal-tumor communication comprising:
comparing the presence, amount, structure, and/or activity of genes or proteins a tumor from an animal model of pancreatic adenocarcinoma, versus the presence, absence, or level of expression or activity of genes or proteins in stroma from an animal model of pancreatic adenocarcinoma, wherein the animal model comprises an activating mutation of KRAS and wherein one or more tumor suppressor genes or loci are misexpressed, and wherein a difference in the amount, structure, and/or activity of a gene or protein indicates that the gene or protein is involved in stromal-tumor communication.

35. A method of assessing whether a subject is afflicted with pancreatic adenocarcinoma, the method comprising comparing:
a) the amount, structure, and/or activity of a biomarker identified in claim 17 or 28 in a subject sample, and
b) the amount, structure, and/or activity of the biomarker in a control pancreatic sample,
wherein a difference in the amount, structure, and/or activity of the biomarker in the subject sample and the normal level is an indication that the subject is afflicted with pancreatic adenocarcinoma.

36. The method of claim 35, wherein the sample comprises cells obtained from the patent.

37. A method for monitoring the progression of pancreatic adenocarcinoma in a subject, the method comprising:
a) detecting in a subject sample at a first point in time, the amount, structure, and/or activity of a biomarker identified by the method of claim 17 or 28;
b) repeating step a) at a subsequent point in time; and
c) comparing the amount, structure, and/or activity detected in steps a) and b), and therefrom monitoring the progression of pancreatic adenocarcinoma in the subject.

38. The method of claim 37, wherein the sample comprises cells obtained from the subject.

39. The method of claim 37, wherein between the first point in time and the subsequent point in time, the subject has undergone surgery to remove a tumor.

40. A method of assessing the efficacy of a test compound for inhibiting pancreatic adenocarcinoma in a subject, the method comprising comparing:
a) the amount, structure, and/or activity of a biomarker in a first sample obtained from the subject and exposed to the test compound, wherein the biomarker is identified by the method of claim 17 or 28, and
b) the amount, structure, and/or activity of the biomarker in a second sample obtained from the subject, wherein the sample is not exposed to the test compound,
wherein a significantly a difference in the amount, structure, and/or activity of the biomarker in the first sample, relative to the second sample, is an indication that the test compound is efficacious for inhibiting pancreatic adenocarcinoma in the subject.

41. The method of claim 40, wherein the first and second samples are portions of a single sample obtained from the subject.

42. A method of assessing the efficacy of a therapy for inhibiting pancreatic adenocarcinoma in a subject, the method comprising comparing:
a) the amount, structure, and/or activity of a biomarker in the first sample obtained from the subject prior to providing at least a portion of the therapy to the subject, wherein the biomarker is identified by the method of claim 17 or 28, and
b) the amount, structure, and/or activity of the biomarker in a second sample obtained from the subject following provision of the portion of the therapy,
wherein a significantly lower level of amount, structure, and/or activity of the biomarker in the second sample, relative to the first sample, is an indication that the therapy is efficacious for inhibiting pancreatic adenocarcinoma in the subject.

43. A method of selecting a composition for inhibiting pancreatic adenocarcinoma in a subject, the method comprising:
a) obtaining a sample comprising cancer cells from the subject;
b) separately exposing aliquots of the sample in the presence of a plurality of test compositions;
c) comparing amount, structure, and/or activity of a biomarker in each of the aliquots, wherein the biomarker is identified by the method of claim 17 or 28; and
d) selecting one of the test compositions which induces a lower level of amount, structure, and/or activity of the biomarker in the aliquot containing that test composition, relative to other test compositions.

44. A method of inhibiting pancreatic adenocarcinoma in a subject, the method comprising:
a) obtaining a sample comprising cancer cells from the subject;
b) separately maintaining aliquots of the sample in the presence of a plurality of test compositions;
c) comparing amount, structure, and/or activity of a biomarker in each of the aliquots, wherein the biomarker is identified by the method of claim 17 or 28; and
d) administering to the subject at least one of the test compositions which induces a lower level of amount, structure, and/or activity of the biomarker in the aliquot containing that test composition, relative to other test compositions.

45. A kit for assessing whether a subject is afflicted with pancreatic adenocarcinoma, the kit comprising reagents for assessing expression of a biomarker identified by the method of claim 17 or 28.

46. A kit for assessing the presence of pancreatic adenocarcinoma cells, the kit comprising a nucleic acid probe wherein the probe specifically binds with a transcribed polynucleotide corresponding to a biomarker identified by the method of claim 17 or 28.

47. A kit for assessing the suitability of each of a plurality of compounds for inhibiting pancreatic adenocarcinoma in a subject, the kit comprising:
a) the plurality of compounds; and
b) a reagent for assessing expression of a biomarker identified by the method of claim 17 or 28.

48. A kit for assessing the presence of human pancreatic adenocarcinoma cells, the kit comprising an antibody, wherein the antibody specifically binds with a protein corresponding to a biomarker identified by the method of claim 17 or 28.

49. A method of assessing the pancreatic cell carcinogenic potential of a test compound, the method comprising:
a) maintaining separate aliquots of pancreatic cells in the presence and absence of the test compound; and
b) comparing amount, structure, and/or activity of a biomarker in each of the aliquots, wherein the biomarker is identified by the method of claim 17 or 28,
wherein a significantly enhanced level of amount, structure, and/or activity of the biomarker in the aliquot maintained in the presence of the test compound, relative to the aliquot maintained in the absence of the test compound, is an indication that the test compound possesses human pancreatic cell carcinogenic potential.

50. A kit for assessing the pancreatic cell carcinogenic potential of a test compound, the kit comprising pancreatic cells and a reagent for assessing expression of a biomarker, wherein the biomarker is identified by the method of claim 17 or 28.

51. A method for identifying a compound which modulates tumor-stromal symbiosis comprising:
(a) administering a test compound to an animal model comprising an activating mutation of KRAS and wherein one or more tumor suppressor genes or loci are misexpressed; and
(b) determining the effect of the test compound on the initiation, maintenance, or progression of pancreatic adenocarcinoma in said animal model, thereby identifying a compound that modulates tumor-stromal symbiosis.

52. A method of identifying a compound that modulates pancreatic adenocarcinoma development, progression, and/or maintenance comprising:
(a) administering a test compound to an animal model comprising an activating mutation of KRAS and wherein one or more tumor suppressor genes or loci are misexpressed, or a cell isolated therefrom; and
(b) determining the effect of the test compound on the initiation, maintenance, or progression of pancreatic adenocarcinoma in said animal model, thereby identifying a compound that modulates pancreatic adenocarcinoma development, progression, and/or maintenance.

53. A method for evaluating a potential therapeutic agent for the treatment or prevention of pancreatic adenocarcinoma comprising:
(a) administering a test compound to an animal model comprising an activating mutation of KRAS and wherein one or more tumor suppressor genes or loci are misexpressed, or a cell isolated therefrom; and
(b) determining the effect of the test compound on the initiation, maintenance, or progression of pancreatic adenocarcinoma in said animal model, thereby evaluating a potential therapeutic agent for the treatment or prevention of pancreatic adenocarcinoma.

54. The method of one of claims 37 or 38, wherein said compound is selected from the group consisting of: a protein, a nucleic acid molecule, an antibody, a ribozyme, an antisense oligonucleotide, an siRNA, and an organic or non-organic small molecule.

55. A method of treating or preventing pancreatic adenocarcinoma in a subject having or at risk of developing pancreatic adenocarcinoma, comprising: administering a compound identified in claim 37 to a subject, thereby treating or preventing pancreatic adenocarcinoma in a subject having or at risk of developing pancreatic adenocarcinoma.

56. An isolated cell, or a purified preparation of cells from an animal model of pancreatic adenocarcinoma comprising an activating mutation of KRAS and wherein one or more tumor suppressor genes or loci are misexpressed.

57. The isolated cell of claim 56, wherein said cell is isolated from pancreatic tissue from said animal model of pancreatic adenocarcinoma.

58. The isolated cell of claim 57, wherein said cell is a epithelial, stomal, acinar, or ductal cell.

59. The cell of claim 56, wherein said cell is transgenic cell.

60. The cell of claim 57, wherein said transgenic cell is a mouse cell.

61. A method of assessing whether a subject is afflicted with pancreatic adenocarcinoma or at risk for developing pancreatic adenocarcinoma, the method comprising comparing the copy number of a minimal common region (MCR) in a subject sample to the normal copy number of the MCR, wherein said MCR is selected from the group consisting of the MCRs listed in Table 2, and wherein an altered copy number of the MCR in the sample indicates that the subject is afflicted with pancreatic adenocarcinoma or at risk for developing pancreatic adenocarcinoma.

62. The method of claim 61, wherein the copy number is assessed by fluorescent in situ hybridization (FISH).

63. The method of claim 61, wherein the copy number is assessed by quantitative PCR (qPCR).

64. The method of claim 61, wherein the normal copy number is obtained from a control sample.

65. A method of assessing whether a subject is afflicted with pancreatic adenocarcinoma or at risk for developing pancreatic adenocarcinoma, the method comprising comparing:
a) the amount, structure, and/or activity of a biomarker in a subject sample, wherein the biomarker is a biomarker which resides in an MCR listed in Table 2; and
b) the normal amount, structure, and/or activity of the of the biomarker,
wherein a significant difference between the amount, structure, and/or activity of the biomarker in the sample and the normal amount, structure, and/or activity is an indication that the subject is afflicted with pancreatic adenocarcinoma or at risk for developing pancreatic adenocarcinoma.

66. The method of claim 65, wherein the amount of a biomarker is compared.

67. The method of claim 65, wherein the structure of a biomarker is compared.

68. The method of claim 65, wherein the activity of a biomarker is compared.

69. The method of claim 66, wherein amount of the biomarker is determined by determining the level of expression of the biomarker.

70. The method of claim 65, wherein amount of the biomarker is determined by determining copy number of the biomarker.

71. The method of claim 65, wherein the normal amount/structure, and/or activity of the biomarker is obtained from a control sample.

72. The method of claims 61 or 65, wherein the sample is selected from the group consisting of blood, urine, stool, bile, pancreatic cells or pancreatic tissue.

73. The method of claim 61 or 70, wherein the copy number is assessed by comparative genomic hybridization (CGH).

74. The method of claim 73, wherein said CGH is performed on an array.

75. The method of claim 69, wherein the level of expression of the biomarker in the sample is assessed by detecting the presence in the sample of a protein corresponding to the biomarker.

76. The method of claim 75, wherein the presence of the protein is detected using a reagent which specifically binds with the protein.

77. The method of claim 76, wherein the reagent is selected from the group consisting of an antibody, an antibody derivative, and an antibody fragment.

78. The method of claim 69, wherein the level of expression of the biomarker in the sample is assessed by detecting the presence in the sample of a transcribed polynucleotide or portion thereof, wherein the transcribed polynucleotide comprises the biomarker.

79. The method of claim 78, wherein the transcribed polynucleotide is an mRNA.

80. The method of claim 78, wherein the transcribed polynucleotide is a cDNA.

81. The method of claim 78, wherein the step of detecting further comprises amplifying the transcribed polynucleotide.

82. The method of claim 69, wherein the level of expression of the biomarker in the sample is assessed by detecting the presence in the sample of a transcribed polynucleotide which anneals with the biomarker or anneals with a portion of a polynucleotide wherein the polynucleotide comprises the biomarker, under stringent hybridization conditions.

83. A method for monitoring the progression of pancreatic adenocarcinoma in a subject, the method comprising:
a) detecting in a subject sample at a first point in time, the amount and/or activity of a biomarker, wherein the marker is a marker which resides in an MCR listed in Table 2;
b) repeating step a) at a subsequent point in time; and
c) comparing the amount and/or activity detected in steps a) and b), and therefrom monitoring the progression of pancreatic adenocarcinoma in the subject.

84. The method of claim 83, wherein the sample is selected from the group consisting of blood, urine, stool, bile, pancreatic cells or pancreatic tissue.

85. The method of claim 83, wherein the activity of a biomarker is determined.

86. The method of claim 83, wherein the amount of a biomarker is determined.

87. The method of claim 86, wherein amount of the biomarker is determined by determining the level of expression of the biomarker.

88. The method of claim 86, wherein the level of expression of the biomarker in the sample is assessed by detecting the presence in the sample of a protein corresponding to the biomarker.

89. The method of claim 88, wherein the presence of the protein is detected using a reagent which specifically binds with the protein.

90. The method of claim 89, wherein the reagent is selected from the group consisting of an antibody, an antibody derivative, and an antibody fragment.

91. The method of claim 87, wherein the level of expression of the biomarker in the sample is assessed by detecting the presence in the sample of a transcribed polynucleotide or portion thereof, wherein the transcribed polynucleotide comprises the biomarker.

92. The method of claim 91, wherein the transcribed polynucleotide is an mRNA.

93. The method of claim 91, wherein the transcribed polynucleotide is a cDNA.

94. The method of claim 91, wherein the step of detecting further comprises amplifying the transcribed polynucleotide.

95. The method of claim 87, wherein the level of expression of the biomarker in the sample is assessed by detecting the presence in the sample of a transcribed polynucleotide which anneals with the biomarker or anneals with a portion of a polynucleotide wherein the polynucleotide comprises the biomarker, under stringent hybridization conditions.

96. The method of claim 83, wherein the sample comprises cells obtained from the subject.

97. The method of claim 83, wherein between the first point in time and the subsequent point in time, the subject has undergone treatment for pancreatic adenocarcinoma, has completed treatment for pancreatic adenocarcinoma, and/or is in remission.

98. A method of assessing the efficacy of a test compound for inhibiting pancreatic adenocarcinoma in a subject, the method comprising comparing:
a) the amount and/or activity of a biomarker in a first sample obtained from the subject and maintained in the presence of the test compound, wherein the biomarker is a biomarker which resides in an MCR listed in Table 2; and
b) the amount and/or activity of the biomarker in a second sample obtained from the subject and maintained in the absence of the test compound,
wherein a significantly higher amount and/or activity of a biomarker in the first sample residing in an MCR which is deleted in pancreatic adenocarcinoma, relative to the second sample, is an indication that the test compound is efficacious for inhibiting pancreatic adenocarcinoma, and wherein a significantly lower amount and/or activity of a biomarker in the first sample residing in an MCR which is amplified in pancreatic adenocarcinoma, relative to the second sample, is an indication that the test compound is efficacious for inhibiting pancreatic adenocarcinoma in the subject.

99. The method of claim 98, wherein the first and second samples are portions of a single sample obtained from the subject.

100. The method of claim 98, wherein the first and second samples are portions of pooled samples obtained from the subject.

101. A method of assessing the efficacy of a therapy for inhibiting pancreatic adenocarcinoma in a subject, the method comprising comparing:
a) the amount and/or activity of a biomarker in the first sample obtained from the subject prior to providing at least a portion of the therapy to the subject, wherein the biomarker is a biomarker which resides in an MCR listed in Table 2, and
b) the amount and/or activity of the biomarker in a second sample obtained from the subject following provision of the portion of the therapy,
wherein a significantly higher amount and/or activity of the biomarker in the first sample residing in an MCR which is deleted in pancreatic adenocarcinoma, relative to the second sample, is an indication that the test compound is efficacious for inhibiting pancreatic adenocarcinoma and wherein a significantly lower amount and/or activity of the biomarker in the first sample residing in an MCR which is amplified in pancreatic adenocarcinoma, relative to the second sample, is an indication that the therapy is efficacious for inhibiting pancreatic adenocarcinoma in the subject.

102. A method of selecting a composition capable of modulating pancreatic adenocarcinoma, the method comprising:
a) obtaining a sample comprising pancreatic adenocarcinoma cells;
b) contacting said cells with a test compound; and
c) determining the ability of the test compound to modulate the amount and/or activity of a biomarker, wherein the biomarker is a biomarker which resides in an MCR listed in Table 2,
thereby identifying a modulator of pancreatic adenocarcinoma.

103. The method of claim 102, wherein said cells are isolated from an animal model of pancreatic adenocarcinoma.

104. The method of claim 102, wherein said cells are from a pancreatic adenocarcinoma cell line.

105. The method of claim 102, wherein said cells are from a subject suffering from pancreatic adenocarcinoma.

106. The method of claim 104, wherein said cells are from a pancreatic adenocarcinoma cell line originating from a pancreatic adenocarcinoma tumor.

107. A method of selecting a composition capable of modulating pancreatic adenocarcinoma, the method comprising:
a) contacting a biomarker which resides in an MCR listed in Table 2 with a test compound; and
b) determining the ability of the test compound to modulate the amount and/or activity of a biomarker which resides in an MCR listed in Table 2, thereby identifying a composition capable of modulating pancreatic adenocarcinoma.

108. The method of claim 102 or 107, further comprising administering the test compound to an animal model of pancreatic adenocarcinoma.

109. A kit for assessing the ability of a compound to inhibit pancreatic adenocarcinoma, the kit comprising a reagent for assessing the amount, structure, and/or activity of a biomarker which resides in an MCR listed in Table 2.

110. A kit for assessing whether a subject is afflicted with pancreatic adenocarcinoma, the kit comprising a reagent for assessing the copy number of an MCR selected from the group consisting of the MCRs listed in Table 2.

111. A kit for assessing whether a subject is afflicted with pancreatic adenocarcinoma, the kit comprising a reagent for assessing the amount, structure, and/or activity of a biomarker which resides in an MCR listed in Table 2.

112. A kit for assessing the presence of human pancreatic adenocarcinoma cells, the kit comprising an antibody or fragment thereof, wherein the antibody or fragment thereof specifically binds with a protein corresponding to a biomarker which resides in an MCR listed in Table 2.

113. A kit for assessing the presence of pancreatic adenocarcinoma cells, the kit comprising a nucleic acid probe wherein the probe specifically binds with a transcribed polynucleotide corresponding to a biomarker which resides in an MCR listed in Table 2.

114. The kit of claim 113, wherein the nucleic acid probe is a molecular beacon probe.

115. A method of treating a subject afflicted with pancreatic adenocarcinoma comprising administering to the subject a modulator of amount and/or activity of a gene or protein corresponding to a biomarker which resides in an MCR listed in Table 2, thereby treating a subject afflicted with pancreatic adenocarcinoma.

116. A method of treating a subject afflicted with pancreatic adenocarcinoma comprising administering to the subject a compound which inhibits the amount and/or activity of a gene or protein corresponding to a biomarker which resides in an MCR listed in Table 2 which is amplified in pancreatic adenocarcinoma, thereby treating a subject afflicted with pancreatic adenocarcinoma.

117. The method of claim 116, wherein said compound is administered in a pharmaceutically acceptable formulation.

118. The method of claim 116, wherein said compound is an antibody or an antigen binding fragment thereof, which specifically binds to a protein corresponding to said biomarker.

119. The method of claim 118, wherein said antibody is conjugated to a toxin.

120. The method of claim 118, wherein said antibody is conjugated to a chemotherapeutic agent.

121. The method of claim 116, wherein said compound is an RNA interfering agent which inhibits expression of a gene corresponding to said biomarker.

122. The method of claim 121, wherein said RNA interfering agent is an siRNA molecule or an shRNA molecule.

123. The method of claim 116, wherein said compound is an antisense oligonucleotide complementary to a gene corresponding to said biomarker.

124. The method of claim 116, wherein said compound is a peptide or peptidomimetic.

125. The method of claim 116, wherein said compound is a small molecule which inhibits activity of said biomarker.

126. The method of claim 125, wherein said small molecule inhibits a protein-protein interaction between a biomarker and a target protein.

127. The method of claim 116, wherein said compound is an aptamer which inhibits expression or activity of said biomarker.

128. A method of treating a subject afflicted with pancreatic adenocarcinoma comprising administering to the subject a compound which increases expression or activity of a gene or protein corresponding to a biomarker which resides in an MCR listed in Table 2 which is deleted in pancreatic adenocarcinoma, thereby treating a subject afflicted with pancreatic adenocarcinoma.

129. A method of treating a subject afflicted with pancreatic adenocarcinoma comprising administering to the subject a protein corresponding to a biomarker which resides in an MCR listed in Table 2 which is deleted in pancreatic adenocarcinoma, thereby treating a subject afflicted with pancreatic adenocarcinoma.

130. The method of claim 129, wherein the protein is provided to the cells of the subject, by a vector comprising a polynucleotide encoding the protein.

131. The method of claim 128, wherein said compound is administered in a pharmaceutically acceptable formulation.

132. An isolated nucleic acid molecule, or fragment thereof, contained within an MCR selected from the MCRs listed in Table 2, wherein said nucleic acid molecule has an altered amount, structure, and/or activity in pancreatic adenocarcinoma.

133. An isolated polypeptide encoded by the nucleic acid molecule of claim 132.

134. The biomarker of claim 22, wherein the biomarker is selected from the group consisting of the biomarker of SEQ ID NO.23 and the biomarker of SEQ ID NO.24.


SEQUENCE LISTING (27 sequences)

SEQ ID NO: 1 - DNA, 713 bp, Mus musculus
gtcacagtgaggccgccgctgagggagtacagcagcgggagcatgggtcgcaggttcttggtcactgtgaggattcagcgcgcgggccgcccactccaagagagggttttcttggtgaagttcgtgcgatcccggagacccaggacagcgagctgcgctctggctttcgtgaacatgttgttgaggctagagaggatcttgagaagagggccgcaccggaatcctggaccaggtgatgatgatgggcaacgttcacgtagcagctcttctgctcaactacggtgcagattcgaactgcgaggaccccactaccttctcccgcccggtgcacgacgcagcgcgggaaggcttcctggacacgctggtggtgctgcacgggtcaggggctcggctggatgtgcgcgatgcctggggtcgcctgccgctcgacttggcccaagagcggggacatcaagacatcgtgcgatatttgcgttccgctgggtgctctttgtgttccgctgggtggtctttgtgtaccgctgggaacgtcgcccagaccgacgggcatagcttcagctcaagcacgcccagggccctggaacttcgcggccaatcccaagagcagagctaaatccggcctcagcccgcctttttcttcttagcttcacttctagcgatgctagcgtgtctagcatgtggctttaaaaaatacataataatgcttttttttt

SEQ ID NO: 2 - DNA, 21 bp, Artificial Sequence
  PCR Primer
tccagaaattggagagttgga

SEQ ID NO: 3 - DNA, 21 bp, Artificial Sequence
  PCR Primer
tcaattccaggtgagacaacc

SEQ ID NO: 4 - DNA, 23 bp, Artificial Sequence
  PCR Primer
tgacagtgtctgtgtgaatccat

SEQ ID NO: 5 - DNA, 22 bp, Artificial Sequence
  PCR Primer
ttaggtgtgtatggtgcagtcc

SEQ ID NO: 6 - DNA, 22 bp, Artificial Sequence
  PCR Primer
acagcactaccacctggactgg

SEQ ID NO: 7 - DNA, 22 bp, Artificial Sequence
  PCR Primer
acaaagaccgcgtggtcactaa

SEQ ID NO: 8 - DNA, 22 bp, Artificial Sequence
  PCR Primer
tttgggtcaggtgccttagtga

SEQ ID NO: 9 - DNA, 21 bp, Artificial Sequence
  PCR Primer
gtccaccatcctggaaatggt

SEQ ID NO: 10 - DNA, 22 bp, Artificial Sequence
  PCR Primer
gtgtcacgcttctccgaagact

SEQ ID NO: 11 - DNA, 23 bp, Artificial Sequence
  PCR Primer
cgtcatgtgctgtgacttcttgt

SEQ ID NO: 12 - DNA, 22 bp, Artificial Sequence
  PCR Primer
gcacgtactctcctcccctcaa

SEQ ID NO: 13 - DNA, 22 bp, Artificial Sequence
  PCR Primer
aggcacaaacacgaacctcaaa

SEQ ID NO: 14 - DNA, 22 bp, Artificial Sequence
  PCR Primer
atgaaccgccgacctatcctta

SEQ ID NO: 15 - DNA, 22 bp, Artificial Sequence
  PCR Primer
ggattgtgctcagccctgaagt

SEQ ID NO: 16 - DNA, 20 bp, Artificial Sequence
  PCR Primer
aggcctgctgaaaatgactg

SEQ ID NO: 17 - DNA, 20 bp, Artificial Sequence
  PCR Primer
ccctccccagttctcatgta

SEQ ID NO: 18 - DNA, 20 bp, Artificial Sequence
  PCR Primer
tgtaaggcctgctgaaaatg

SEQ ID NO: 19 - DNA, 20 bp, Artificial Sequence
  PCR Primer
gcacgcagactgtagagcag

SEQ ID NO: 20 - DNA, 2629 bp, Mus musculus
acttgtcatggcgactgtccagctttgtgccaggagcctcgcaggggttgatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggtaggggcccacttcaccgtactaaccagggaagctgtccctcatgttgaattttctctaacttcaaggcccatatctgtgaaatgctggcatttgcacctacctcacagagtgcattgtgagggttaatgaaataatgtacatctggccttgaaaccaccttttattacatggggtctaaaacttgacccccttgagggtgcctgttccctctccctctccctgttggctggtgggttggtagtttctacagttgggcagctggttaggtagagggagttgtcaagtcttgctggcccagccaaaccctgtctgacaacctcttggtcgaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtggagctggaagggtcaacatcttttacattctgcaagcacatctgcattttcaccccacccttcccctccttctccctttttatatcccatttttatatcgatctcttattttacaataaaactttgctgcca

SEQ ID NO: 21 - DNA, 3361 bp, Mus musculus
ccgctgcggtaacggagcggctcgggtggcggagcccgtgttcgcgtccgtccgcccgcccgcccgccgtcctccggaggcccttcccgcgccgcgctccgctccgcggccgtccccggggcgggagcgcgtgaccggagccggcgcccgcgagcgaggccccccgcagcggggcggctccggagctccagcggcccggccggccggcgcggtccgcggcgcggcggggagagggggccgcctgggccggacgccgcgggcggggcccgggaagcgacagcgaggcgaggcgcggtgcggcgcggagcccaggtcatcctgctcaccagatgtcttgacagtttttcttgcaacattggccattggttttcactgccttcaaaagatcaaaattactccagaaattggagagttggatttaaaagaaaaaacttgaacaaatggacaatatgtctataacaaatacaccaacaagtaacgatgcctgtctgagcattgtacatagtttgatgtgtcatagacaaggtggggaaagtgaaacctttgcaaaaagagcaattgagagtttggtaaagaagctgaaagagaaaaaagatgaattggattctttaataacagctataactacaaatggagctcatcctagcaagtgtgtcaccatacagagaacattggatggacgacttcaggtggctggtcggaaaggatttcctcatgtgatctatgcccgtctgtggaggtggcctgatctacacaagaatgaactaaagcatgttaaatattgtcagtatgcgtttgacttaaaatgtgacagtgtctgtgtgaatccatatcactatgagcgggttgtctcacctggaattgatctctcaggattaacactgcagagtaatgctccaagtatgttagtgaaggatgagtacgttcacgactttgaaggacagccgtccttacccactgaaggacattcgattcaaaccatccaacacccgccaagtaatcgcgcatcaacggagacgtacagcgccccggctctgttagccccggcagagtctaacgccaccagcaccaccaacttccccaacattcctgtggcttccacaagtcagccggccagtattctggcgggcagccatagtgaaggactgttgcagatagcttcagggcctcagccaggacagcagcagaatggatttactgctcagccagctacttaccatcataacagcactaccacctggactggaagtaggactgcaccatacacacctaatttgcctcaccaccaaaacggccatcttcagcaccacccgcctatgccgccccatcctggacattactggccagttcacaatgagcttgcattccagcctcccatttccaatcatcctgctcctgagtactggtgctccattgcttactttgaaatggacgttcaggtaggagagacgtttaaggtcccttcaagctgccctgttgtgactgtggatggctatgtggatccttcgggaggagatcgcttttgcttgggtcaactctccaatgtccacaggacagaagcgattgagagagcgaggttgcacataggcaaaggagtgcagttggaatgtaaaggtgaaggtgacgtttgggtcaggtgccttagtgaccacgcggtctttgtacagagttactacctggacagagaagctggccgagcacctggcgacgctgttcataagatctacccaagcgcgtatataaaggtctttgatctgcggcagtgtcaccggcagatgcagcaacaggcggccactgcgcaagctgcagctgctgctcaggcggcggccgtggcagggaacatccctggccctgggtccgtgggtggaatagctccagccatcagtctgtctgctgctgctggcatcggtgtggatgacctccggcgattgtgcattctcaggatgagctttgtgaagggctggggcccagactaccccaggcagagcatcaaggaaaccccgtgctggattgagattcaccttcaccgagctctgcagctcttggatgaagtcctgcacaccatgcccattgcggacccacagcctttagactgagatctcacaccacggacgccctaaccatttccaggatggtggactatgaaatatactcgtgtttataatctgaagatctattgcattttgttctgctctgtcttttcctaaagggttgagagatgtgtttgctgccttgctcttagcagacagaaactgaattaaaacttcttttctattttagaactttcaggtgtggctcagtgcttgaagatcagaaagatgcagttcttgctgagtcttccctgctggttctgtatggaggagtcggccagtgctgggcgctcagccctttagtgtgtgcgagcgccttgcatgccgaggagagtcagagctgctgattgtaaggctgagaagttctcacagttaagccacctgccccttagtgggcgagttattaaacgcactgtgctcacgtggcgctgggccagccagctctaccaagagcaactttactctcctttaaaaaccttttagcaacctttgattcacaatggtttttgcaagttaaacagtgaaggtgaattaaattcatactgtcttgcagacttcagggtttcttccccaagacaaaacactaatctgtgtgcatattgacaattccttacaattatcagtcaaagaaatgccatttaaaattacaatttttttaatccctaatggatgaccactatcaagatgtatactttgccctgttaaacagtaaatgaattcttctatatttctaggcacaaggttagttatttaaaaaaaaaaaaaaaagcctaggggagggatttttcccttaattcctagggagaaggttttgtataaaacactaaaagcagtgtcactctgcctgctgcttcactgttctgcaaggtggcagtacttcaactgaaataatgaatattttggaaactgctaaattctatgttaaatactgtgcagaataatggaaacagtgcagttggtaacaggtggtttggatatttttgtacttgatttgatgtgtgacttcttttcatatactgttaaaatcatgtatgttttgacattgtttaaaattcagtttttgtatcttagggcaagactgcagacttttttataccttttggttataagccctgtgtttgccatccttgatcacttggcggtgactttgtagagattgaagtggaggagttaagacacattgactgtaccacagacacacatgtatactttctacctagttactagcgtaaataaaactgagtcactataaaaaaaaaaaaaaaaaaaaa

SEQ ID NO: 22 - DNA, 2177 bp, Mus musculus
ctgcggccgctgggcccctgagcgcggggcccaagtggcgaacatgacctagcggcccgcgcgccgcgacggcggaccgtcgcctccgtcggaagcagcgtccccggggacccgaattggggggacgcgagggttgggggggctcattgcttttttttttttcattcttattttcattttttttctccctgagcacctagaagaaaaggggaaaatcaaaagtgaagaattggcgcccaggaagcggacgtggacccggttgtggggaccgggagagttgtggaggtcgttcctgtttttccccgttccttttttccccttcttggaacatttggaagaagcgggtgggggggggggaattcgaacttgaaaagaattggcgctcccgaaggggacgaggacaaagagtgggccaggatggacgtggcggaccccgagccgttgggccttttctccgagggcgagctgatgtcggtgggcatggacaccttcatccaccgcatcgactccaccgaggtaatctaccagccgcgccgcaaacgcgccaagctcatcggcaagtacctgatgggggacctgctcggggagggctcgtacggcaaggtgaaggaggtgctggactccgagaccttatgccgcagggcggtcaagatcctcaagaagaaaaagctgcgcaggatccccaatggagaggccaacgtcaagaaggagatccagctgctgcggcggctgcggcatcggaatgtgatccagcttgtggacgtgctgtacaatgaggagaagcagaagatgtatatggtgatggagtactgcgtatgtggcatgcaggagatgctggacagtgtgccggagaagcgcttccctgtgtgccaagctcatgggtacttccgccagctgattgacggcctggaatacctacacagccagggcattgttcacaaggacatcaagccgggcaacctgctactcaccaccaatggcacactcaagatctccgacctcggtgttgccgaggccctgcaccctttcgctgtggatgacacctgccggacaagccagggctccccggccttccagcctcctgagattgccaatggactggacaccttttcaggtttcaaggtggacatctggtcagctggggtcacactttacaacatcaccacgggcctgtacccatttgagggggacaatatctacaagctctttgagaacattgggagaggagacttcaccatcccttgtgactgcggcccaccactctctgacctactccgagggatgttggagtatgagccggccaagaggttctccatccgacagattaggcagcacagctggttccggaagaaacaccctctggctgaggcgctcgtacctatcccaccaagcccagacactaaggaccgctggcgcagtatgactgtagtgccctacctggaggacctgcatggccgtgcggaggaggaggaggaggaagacttgtttgacattgaggacggcattatctacacccaggacttcacagtgcctggacaggtcctggaagaggaagtgggtcagaatggacagagccacagtttgcccaaggctgtttgtgtgaatggcacagagccccagctcagcagcaaggtgaagccagaaggccgacctggcaccgccaaccctgcgcgcaaggtgtgctccagcaacaagatccgccggctctcggcctgcaagcagcagtgactgaggcctacagtgtgtcatcaggatctctgggcaggtgtccctgcaaggctgggttttccaggcctgcctgtccactcacttcgggacgttggagccgagggcggacctgctgccccagaagcactttatgtcgagaccactggccggccttgcctgcatgccgccctgcgagcctcgctgtctttgggttggtttctttttttttaataaaacaggtggatttgagctatggctatgagggtgtttggaaatatggagcaggcggggcacagggtggcctgcagagaaaacccagagcaaacaaatatgcagagacatttatgattaaccagacaacacgaccaaccacagagggcgcagggcagggagtgggcaggcactcacagcgagtctgccctatcttttggcaataaataaagcttgggaaacttgaaaaaaaaaaaaaa

SEQ ID NO: 23 - protein, 11 aa, Mus musculus
HLGSVTALHVL

SEQ ID NO: 24 - protein, 8 aa, Mus musculus
QEQERKEK

SEQ ID NO: 25 - DNA, 2548 bp, Mus musculus
gctcgagtgattggctgacgggcaactctggcgccaatatggcgtttgtagcaggagttattcggcgtctggacgagacggtggtgaaccgcatagcggcgggggaagtcattcagcggccggccaatgctatcaaagagatgatagaaaactgtttagatgcaaaatctacaaatattcaagtggttgttaaggaaggtggcctgaagctaattcagatccaagacaatggcactggaatcaggaaggaagatctggatattgtgtgtgagaggttcactacgagtaaactgcagacttttgaggatttagccagtatttctacctatggctttcgtggtgaggcattggcaagcataagccatgtggcccatgtcactattacaaccaaaacagctgatgggaaatgtgcgtacagagcaagttactcagatggaaagctgcaagcccctcctaaaccctgtgcaggcaaccagggcaccctgatcacggtggaagaccttttttacaacataatcacaaggaggaaagctttaaaaaatccaagtgaagagtacggaaaaattttggaagttgttggcaggtattcaatacacaattcaggcattagtatctcagttaaaaaacaaggtgagacagtatctgatgtcagaacactgcccaatgccacaaccgtggacaacattcgctccatctttggaaatgcggttagtcgagaactgatagaagttgggtgtgaggataaaaccctagctttcaaaatgaatggctatatatcgaatgcaaactattcagtgaagaagtgcattttcctactcttcatcaaccaccgtctggtagaatcagctgccttgagaaaagccattgaaactgtatatgcagcatatttgcccaaaaacacacacccattcctgtacctcagtttggaaatcagccctcagaacgtggacgtcaatgtacaccccaccaagcacgaagttcactttctgcatgaggagagcattctgcagcgtgtgcagcagcacattgagagcaagctgctgggctccaattcctccaggatgtatttcacccagaccttgcttccaggacttgctgggccctctggggaggcagctagacccacgacaggggtggcttcctcatccactagtggaagtggcgacaaggtctacgcttaccagatggtccgtacggactcccgggatcagaagcttgacgcctttctgcagcctgtaagcagccttgtgcccagccagccccaggaccctgcccctgtccgaggggccaggacagagggctctcctgaaagggccacgcgggaggatgaggagatgcttgctctcccagcccccgctgaagcagctgctgagagtgagaacttggagagggaatcactaatggagacttcagacacagcccagaaagcggcacccacttccagtccaggaagctccagaaagagacatcgggaggactctgatgtggaaatggtggaaaatgcttccgggaaggaaatgacagctgcttgctaccccaggaggaggatcattaacctcaccagcgtcttgagtctccaggaagagattagtgagcggtgccatgagactctccgggagatgctccgtaaccattcctttgtgggctgtgtgaatcctcagtgggccttggcacagcaccagaccaagctatacctcctcaacactaccaagctcagtgaagagctgttctaccagatactcatttatgattttgccaactttggtgttctgaggttatcggaaccagcgccactcttcgacctggccatgctggccttagacagtcctgaaagtggctggacagaggacgacggcccgaaggaagggcttgcagagtacattgtcgagtttctgaagaagaaagcggagatgcttgcagactatttctctgtggagatcgatgaggaagggaacctgatcggattacctcttctgattgacagctatgtgccacctttggagggactgcctatcttcattcttcgactggccactgaggtgaattgggatgaagaaaaggagtgttttgaaagtctcagtaaagaatgtgctatgttttactccattcggaagcagtatatactggaggagtcgaccctctcaggccagcagagtgacatgcctggctccacgtcaaagccctggaagtggactgtggagcacattatctataaagccttccgctcacacctcctacctccgaagcatttcacagaagatggcaatgtcctgcagcttgccaacctgccagatctatacaaagtctttgagcggtgttaaatacaatcatagccaccgtagagactgcatgaccatccaaggcgaagtgtatggtactaatctggaagccacagaataggacacttggtttccaagctccagggtttccagtgctcactattcttgttctgtatcccagtattggtgctgcaacttaatgtacttcacctgtggattggctgcaaataaactcacgtgtattggaaaaaaaaaaaaaaaaaaaaaaa

SEQ ID NO: 26 - DNA, 10276 bp, Mus musculus
acttccttaccgagcatcggagaaatactgtagagaaaatgcccgttgaatacaaaaggagaccaactttttgggaaatttttaaggcgagatgcagcacagcagatttaggaccgataagcctcaattggtttgaggagctttcctcagaagcccccccatacaattttgaacctccggaggaatctgagtataagccccacggttatgaaccacagctgtttaaaacaccacagaggaatcccccctaccatcagtttgcttcaactccaataatgttcaaagaacgaagtcaaactctaccactggaccagtcgcctttcagagaattagggaaggttgttgcaagtagtaaacataaaactcacagcaaaaagaagaccaaagtggaccctgtggtagatgttgctagtccgcctctgaaatcttgtctcagtgaaagccctcttactttgcggtgcacacaggcagtactacaacgagaaaagccagtggtatctggaagtttattttatacaccaaaactcaaggagggtcagacaccaaaacctatttctgagagtctgggagttgaagtggatcctgatatgtcttggacaagctcattagctacaccaccaacccttagttccactgtgctcatagcccgagatgaagaagcacgcagctctgtaacgcctgctgactctcccgctactttgaaaagctgtttttctaaccacaatgaaagtccacaaaaaaatgatagatctgttccctcagtgattgacagtgaaaacaaaaaccagcaagaagcttttagtcagggtttggggaaaatgttaggagattcatctggcaaaagaaatagcttcaaagactgccttagaaagccaataccaaatattctagaagatggggagacagctgttgatacttccgaagaagatagtttctcattatgttttcctaagcgtagaaccagaaatctacagaaaatgagaatgggcaagacaaggaaaaaaattttcagtgaaacaagaactgatgaattaagcgaagaagctagaagacaacctgatgataaaaactcatttgtatttgaaatggaactaagagagagtgatcctttagatccaggtgttacaagccagaagcccttttacagccagaatgaggaaatctgcaatgaagctgtacagtgttcagatagtagatggtctcagtcaaacctttctggtctaaatgaaacccagacaggaaaaataaccttacctcatatttcttctcatagccaaaatatttcagaagacttcatagatatgaagaaagaaggtactggctctattacttcagaaaaatctttacctcatatttctagtcttccagaaccggaaaagatgttcagtgaggagactgtggtagataaggaacatgaaggacagcattttgaatcacttgaagactccattgcagggaagcaaatggtgtctagaacaagtcaagcagcctgtctttctccgagtatcaggaagtctatattcaagatgagagagccacttgacgagactttgggtactgttttctcagatagtatgaccaactcaacctttacagaagaacatgaagcctctgcatgtggattgggaatacttactgcatgctcacagagagaggattctatatgtcctagttcagttgacactggaagctggccaaccactctcactgacacttctgccactgtgaaaaatgcaggtttgatatccactctaaaaaataaaaaaagaaagtttatttactctgtaagtgatgatgcatctcttcaaggaaaaaaactacagacacacagacagttagagcttactaatctttcagcccagcttgaagcaagtgcttttgaagtaccattgacctttacaaatgtaaattcaggtataccagattcttctgacaaaaaacgctgtttaccgaatgatcctgaagagccatccttgaccaactcttttgggactgctaccagtaaagaaattagttatattcatgcattgatatctcaggatcttaatgacaaagaagcaatagtcattgaagaaaaaccacagccatatacagcccgagaagctgactttctgttatgcttgccagaaagaacatgtgaaaatgaccaaaaaagtccaaaagtttccaatggaaaagaaaaagtcttagtttcagcatgtcttccttcagcagtgcaactcagcagcattagctttgaatcacaggaaaaccctcttggtgaccacaacggaacaagtactcttaaattaactcccagctcaaagctacctctgtcaaaggcagacatggtttctagagagaaaatgtgtaaaatgccagagaaactgcaatgtgagagttgtaaagttaatattgaactaagtaaaaacatcttggaggtaaatgaaatatgtatcttaagtgaaaattctaaaactcctgggcttctgccacctggtgaaaatataatagaagtggcctcttcaatgaagtcacagttcaatcaaaatgcaaaaatagtcatacagaaggaccaaaaaggctcaccttttatttcagaagtagcagtcaatatgaactctgaagaacttttcccagacagtgggaataattttgcttttcaagtaactaataaatgcaataagcctgatttaggaagttcagtggaactccaggaagaagacctcagccacacacaagggcctagtctcaagaactctcccatggcagtagatgaagatgtagatgatgcgcatgcagcccaagtgttgattacaaaggactcagattcattagctgtggtccatgattatacagagaagagcagaaataatatagagcagcaccagaaaggaaccgaagacaaagatttcaagtcaaattcctccttgaatatgaaatcagatgggaacagtgattgttcagacaaatggtcagagttcttggatccagtcttgaaccataattttggaggtagcttcagaacagcttccaataaagaaataaaactttcagagcataatgtcaagaaaagtaaaatgttcttcaaagatattgaagaacagtatcctactaggttagcttgtattgacattgttaatacccttccattagcaaaccagaagaaactaagtgaacctcatatatttgatttgaaatcagttactactgtatctacacagtctcacaatcaatcatctgtttctcatgaagatactgacacagcacctcagatgttatcttcaaagcaagattttcattcaaataatttaacgaccagccaaaaagcagaaattacagaactgtctactattttggaagaatcaggaagtcagtttgaattcacacagttcagaaagccaagccacatagcacagaatacatctgaagtgcctggaaaccagatggttgttttaagtaccgcttctaaggagtggaaagatactgatcttcacctcccagtggatccctctgtaggtcagacagatcacagcaagcaatttgaaggttcggctggagttaaacaaagctttcctcacctgttagaagacacttgtaacaaaaatacatcttgttttttaccaaatataaatgaaatggagtttggaggattttgttctgctcttggcacaaaacttagtgtgtctaatgaggctctgagaaaagctatgaaactgttcagtgacattgaaaatagtgaggagccttctgcaaaagtaggaccaagaggattctcttcaagtgcacaccatgattctgttgcttcagtgtttaagataaagaaacaaaacactgaaaaaagttttgatgaaaaatctagtaagtgccaagtaacattacaaaataatattgaaatgactacctgtatttttgttggcagaaatcctgaaaaatacataaagaatacaaaacatgaagatagctatactagctctcaaagaaataatttagaaaactctgatggtagtatgtcaagtacaagtggcccagtttatattcataaaggtgacagtgatttacctgccgatcaaggcagtaagtgtcctgagtcatgtacccaatatgcgagagaggaaaacacacaaattaaggaaaatatatcagatttaacatgtttggaaattatgaaagctgaggaaacatgtatgaaatcttcagataaaaaacaattaccttcagataagatggaacaaaatataaaagagtttaatatatcctttcagactgcaagtgggaaaaataccagagtctccaaagagtcattaaataaaagtgtgaatatttttaatcgggaaacagatgaattgactgtcatttcagattctttgaattctaaaattctccatggcataaataaggacaaaatgcatacttcatgtcacaagaaagcaatcagtattaaaaaggtatttgaagaccatttcccaattgtaactgtcagtcaattaccagctcagcagcatcctgaatatgaaatagaaagtaccaaagaacctactctgttgagttttcatacagctagtgggaaaaaagtcaaaattatgcaggaatctttggacaaagtgaaaaacctttttgatgagacacaatatgttaggaaaactgccagttttagtcaaggatcaaaacccctgaaggacagtaaaaaagaacttacattagcatatgagaaaattgaagtaactgcctcaaaatgtgaagaaatgcagaactttgtctctaaggagactgaaatgctacctcagcaaaattatcatatgtataggcaaactgaaaatctcaaaacatcaaatggtacctcttccaaagtacaagaaaacatagaaaataatgtagaaaagaatcctagaatttgctgtatttgtcagtcctcttacccagtcactgaagattctgctttggcatattatacggaggacagtaggaaaacttgtgtcagagagtcttctctatccaaaggcagaaaatggcttagagaacagggtgataagcttggaacaagaaatactatcaaaattgagtgtgtaaaggaacacacagaagattttgcaggaaatgcctcatatgaacatagtttggtcattatcagaactgaaattgatacaaatcatgtctctgaaaaccaagtgtcaaccctccttagtgaccctaatgtgtgtcatagctatctatcccagtctagtttttgtcattgtgatgacatgcataatgattcaggatatttcttaaaaaataaaattgattctgacgttccgccagacatgaggaatgctgaaggcaataccatttcccccagagtatctgctacaaaagaaagaaatctacacccacaaactataaatgaatattgtgttcagaaactggagactaatacttcaccacatgcaaataaagatgtagccattgacccatttttgctggattcaaggaattgtaaggtaggctcactcgtgtttattacagctcattcacaagaaactgaaagaacaaaagagatagtcacagataactgttataaaatagttgagcaaaacagacagagtaaaccagacacttgccagacaagctgtcataaagtattggatgattcaaaggattttatatgtcctagctcttcaggtgatgtttgcataaactcacgtaaggatagtttttgtcctcataatgaacaaattttacaacataaccaaagtatgtttggactgaagaaagctgcaacaccacctgttggtttggaaacttgggatacaagtaaatctataagagaacctccccaggcagcccatccttcacgcacttatgggatttttagcacagcaagtggaaaagctatacaagtatcagatgcttcattagaaaaggcaaggcaagtgttttctgagatggatggtgatgctaaacagttatcttccatggtgtcactggaaggtaatgaaaaaccacatcactctgtgaaaagagaaaactctgtggtgcatagcacccagggtgtattgtcactcccaaaacccctcccaggcaatgtcaattcatctgtattctctggatttagcactgcaggtggaaaactggtcacagtttcagaaagtgccttacacaaagttaaaggaatgttagaggagtttgatttgatcagaactgaacatactctccagcattcacctatacctgaagacgtatcaaaaatacttcctcaaccttgtgctgaaatcagaaccccagaataccctgtaaactcaaaattgcagaaaacctacaatgataaatccagcttaccaagtaattataaagaaagtggttcttcgggcaatactcaatctattgaagtttctctccaactctctcagatggagagaaaccaagacacacagttggtattaggaacaaaagtatcccatagtaaggctaatcttttgggaaaagaacaaactttaccccaaaacataaaagtaaaaactgatgaaatgaaaacattttctgatgttcctgtgaaaacaaatgtaggagagtattactccaaagagtcagagaactattttgaaacagaagcagtggagagtgccaaagcttttatggaagatgatgagctgacagattctgaacagactcatgccaaatgctcactgtttacatgcccccaaaatgagactttgtttaattcgagaactaggaaaagaggaggagtgactgttgacgcagttggacaacccccaatcaaaagaagcttattaaatgaatttgacaggataatagaaagtaaaggaaaatccttaacaccttcaaaaagcactccagatggcacagtaaaagacagaagcttgttcacacaccacatgtctttagagccggttacctgtggacccttctgctcaagtaaagaaaggcaaggagcccagcgcccacatcttacctcacctgctcaagagcttctgtctaaagggcatccctggagacactcagctttggaaaagtctccaagcagtcctatagtttcaatcttaccggctcacgacgtttctgccacaagaaccgaaaggaccagacattcaggcaaatccaccaaagtcttcgtcccgcctttcaagatgaaatcacagtttcacggagatgaacatttcaatagcaagaacgttaatttggagggaaagaaccaaaagagcacagatggagacagggaagacgggaacgacagccatgttcgtcagtttaacaaagatttaatgtcaagccttcagagtgccagagacctacaggatatgcgaattaaaaacaaagaaaggcgacatctccgtctgcagccaggcagtctgtatcttacaaagtcatccaccctgccccggatctccctgcaggctgcagtaggagacagggctccttctgcatgttctcctaagcagctctatatatacggcgtttctaaagagtgcataaacgttaatagcaaaaatgctgagtattttcagtttgatattcaggatcattttggtaaagaagatctgtgtgctggcaaaggctttcagttggcagatggtggatggctgatcccctccaatgatggcaaggctggaaaagaagaattttatagggctctgtgtgacactccaggtgtggatccaaagcttatttctagcatctgggttgctaaccattacaggtggattgtatggaaactggcagctatggaatttgcttttcctaaggaatttgctaatagatgcctaaacccagaaagagtgctgcttcaactgaaatacagatacgatgtggaaattgacaacagcaggagatcggctctaaagaagatactggagagggatgacacagctgcaaaaacccttgttctctgtatttctgacatcatttcaccaagcacaaaggtgtctgaaacttcaggcggtaaaactagtggtgaggatgccaacaaagtggacacgatcgaactcacagatgggtggtacgctgtcagggcccagctagaccctccactcatggctcttgtaaagagtggcaaactgactgtgggtcagaagatcattactcagggagcagagctggtgggctctcctgatgcctgtgcacctctggaagccccagactccctaaggctaaagatttctgcaaacagcacaaggcctgctcgctggcacagcaggctggggttcttttgtgaccccaggcccttccctttgcccttgtcctcaatgttcagtgacggaggaaatgttggctgtgtggatatcattgttcagagagtgtaccctttacagtgggtggagaagacagtgtctggattgtacatatttcgtagtgagagagaagaggagaaggaagcactgagatttgcagaggcccagcagaagaaactagaggccttgttcaccaaagtccacacagagtttaaagaccacgaagaagacacaacacagcggtgtgtgctgtcccgcacactcacacggcagcaagtccatgctctgcaggatggagcagagctctatgcagcagtgcagtatgcatcagacccagatcaccttgaggcttgtttcagcgaagagcagctaagagccttgaacaactacagacaaatgctgaatgataagaaacaagcgcggatccagtcagaattccggaaggccctggaatccgctgagaaagaggagggtttatcaagggatgttacaactgtgtggaagctgcgggttacaagctacaagaaaaaggaaaaatcagctctgttgattatctggcgtccatcatcagacttatcctccctgttaacagaaggaaagagatacagaatctatcttcttgcagtgtcaaaatcgaaaagtaaatttgaacggcccagcatccagttaacagccacaaaaagaactcagtatcaacagctgccggtttccagtgagactttactccaggtttaccagcccagggagtcccttcacttcagcagactgtcagacccagcctttcagcctccttgttctgaagtggatgtcgtaggagttgtagtttctgttgtaaaaccgataggtcttgctcctttggtctacctgtcagacgagtgccttaatttattagtggtgaaattcgggatagaccttaatgaagacataaagccacgtgtgctaattgccgcaagcaacctccagtgccagccagaatccacatcaggagtgccgaccttatttgctggccatttttccatattttctgccagtccaaaggaggcctactttcaggagaaagtcaacaacttgaagcatgctattgagaatatcgatacattttacaaggaggcagaaaagaagcttatacacgtgcttgagggagacagtcccaagtggtccacaccgaacaaagaccccacccgagagccccatgcagcctccacttgctgtgcttcagatctccttggttcaggaggtcagttcctgaggatttcacctactggtcagcaaagttatcaaagtcctttatcacactgcacactgaaagggaagtctatgcccctggctcactcagcccagatggcagcaaagtcttggagtggagagaacgagattgatgacccaaaaacctgtcgaaaaagaagagccttggatttcctaagtcggctgcccttaccgtccccggtcagtcccatctgtacctttgtctctccagctgcacagaagggttttcagccgccacggagctgtggcacgaaatacgcaacacctattaaaaaaaaacccagttccccgcggaggaaaacgccatttcagaagaccagtggcgtttctctgccggactgtgattcagtagctgatgaagaacttgccttgctcagcacccaggcccttacgcctgactccgtgggaggaaatgaacaagcgtttcccggtgattccacaaggaacccccagccagcacagaggccagaccagcaagtagggcccaggtccaggaaggagtctctcagggactgtagaggcgacagcagtgagaaattagctgttgagtcttagcctcccggtttgtaagatgtgtacagttccaggctactcataggtgttcgtgtgagaaggcgggagtagctgcactctactgaagcgtgtgtttactgctcagatgctctacctggtctgtgttggtgtaactaaagctggttgtcatcgactgatttaaaaagctttaggagtagactggatcactacaactatcattttatatacaaaataaacatagctttgcttttagtttaaaaaaaaaaaaa

SEQ ID NO: 27 - DNA, 1785 bp, Mus musculus
ccacgcgtccgcccacgcgtccggcagccgccgcggaggtctttggccagtatcgattccacctgtccacaaacatggggaatatctttgcaaacctcttcaagggcctttttggcaaaaaagaaatgcgcattctcatggtgggcctggatgctgcagggaagacaacaattctatacaaacttaagctgggcgaaattgtgaccaccattcccaccattggtttcaatgtggagactgttgaatacaagaatatcagcttcaccgtgtgggatgtgggcggccaggacaagatccggccgctgtggcgccactacttccagaacacccaaggcttgatcttcgtagtggacagcaatgacagagagcgtgtgaacgaggcccgtgaagagctcatgaggatgctagctgaagatgagctccgagatgctgttctcttggtgtttgccaacaagcaggacctccccaatgccatgaatgcggccgaaatcacagacaagctggggctgcactctctacgccacaggaactggtacattcaggccacctgtgccaccagcggggacgggctctatgaaggactagattggctgtctaatcagctccggaaccagaagtgaaccagacccctccctccccctcacttcctcttctccgccctcagctttcctctcatgtggcaaacgtgcgactctgtggtcctgagtgccagaagctgtctccatgggttggtcacagtgtgcatcgcaccgtgctgtacatgtgcagacgcagcctgcagccaggtttttatttaatgtaaatagtttctgtttccactgaggcagtttctggtactcctatgcaatattacttagcttttttattgtaaaaagagaatcaactcaactgtcagtactgagaagggatttgggtgtaggggcacctggcctccgggagccattgggctgtagactggtgtcggtatccatttggtggttggtttttaacccaaactcagtgcattttttaaaaatagttaaaaatacaggacaagaacacttgaacacacagaacggagactatgcctagtgtaggttttgcagttaatggcctgaatgctagttatcagatcacctgtttcgctgtgggaacaggagagaaggtgatgaacaaaccaccatccgctgcatggtcacagtagagcccccgtgactcgcctgtctttgggtcacctgcattccatagcattgtgcttgtacttgtgctcacacggttacctagggtaggctgggagccattgtggggtgcagggcctggcttgtacttggtgtgtgcaaggcccaatggcagcctgcatacccagcctactcttgggcccacttggacgcgctggcaggaggcctgggtctcaccagcaggagtgcgtgcaaggtgggagggtcggtccattacagacccacatcctggagcacccccatctccatgtgtgaagtagcttcctccctcagcctgcaagggtccgatttgccatcgaaagacgacctctacttttttcttttgtattttgataaacactgaagaagctggagctgttaaatttatcttggggaaatctcagaactggtttatttggtgtcgtggaacctcttaccgctttcaatacacaattagtaatcaactgttttgtatacttgttttcagttttcatttcgacaagcactgtaattatagctgttagaataaagtctcttaactatttaaaaaaaaaaaaaaaaaaaaaaaaaaaaaaaaaaaaaaaaaaaa